(19) United States
(12) Patent Application Publication
(10) Pub. No.: US 2003/0091973 A1
(43) Pub. Date: May 15, 2003

(54) Method of identifying osteoregenerative agents using differential gene expression

(76) Inventors: Gregory J. Horesovsky, Morrisville, NC (US); L. Staton Noel III, Durham, NC (US); Debasish Raha, New Haven, CT (US)

Correspondence Address: Ivor R. Elrifi, Ph.D., MINTZ, LEVIN, COHN, FERRIS,, GLOVSKY AND POPEO, P.C., One Financial Center, Boston, MA, 02111, US

(21) Appl. No.: 09/956,622

(22) Filed: Sep. 19, 2001

Related U.S. Application Data
(60) Provisional application No. 60/233,579, filed on Sep. 19, 2000.

(51) Int. Cl. C12Q 1/00; C12Q 1/68; G01N 33/53; A61K 49/00

(52) U.S. Cl. 435/4; 435/6; 435/7.1

(57) ABSTRACT

Disclosed are methods of identifying osteoregenerative agensts using differential gene expression. Also disclosed are novel nucleic acid and polypeptide sequences whose expression is differentially regulated by parathyroid hormone.

### RELATED U.S. APPLICATIONS

This application claims priority to U.S. Ser. No. 60/233,579 filed Sep. 19, 2000, which is incorporated herein by reference in its entirety.

### FIELD OF THE INVENTION

The invention relates generally to the identification of osteoregenerative agents using differential gene expression.

### BACKGROUND OF THE INVENTION

Intermittent administration of parathyroid hormone (PTH) is known to cause an increase in skeletal mass, whereas chronic administration has the opposite effect, however the molecular mechanisms underlying this phenomenon are poorly understood.

Bone continuously remodels in response to mechanical and physiological stimuli by replacing old bone with new one. Bone remodeling consists of tightly coupled cycled degradation and synthesis of extra cellular bone matrix proteins. Bone remodeling is carried out mainly by two types of cells present in the bone tissues, osteoblasts, the bone forming cells and osteoclast, the bone resorbing cells. A change either in the number or activity of these two types of cells can lead to bone diseases like osteopenia, osteopetrosis and osteoporosis. Osteoporosis is a disease characterized by low bone mass and deterioration of bone tissue. This leads to increase in bone fragility and risk of fracture, particularly of the hip, spine and wrist. Osteoporosis is a major health problem and affects women at their post-menopausal decade and also men. All the FDA approved drugs inhibit further bone loss but do not help in replacement synthesis of lost bone. Thus there is a need for specifying with precision the factors involved in restoration of lost bone mass such as occurs in osteoporosis, especially those factors responsive to PTH. There is further a need for detecting the response of factors to PTH administration, and for monitoring the efficacy of intermittent treatment of a subject with parathyroid hormone.

### SUMMARY OF THE INVENTION

The invention is based in part on the discovery that certain nucleic acids are differentially expressed in tibiae cells of animals treated with parathyroid hormone. These differentially expressed nucleic acids include previously undescribed sequences and nucleic acids sequences that, while previously described, have not heretofore been identified as parathyroid hormone responsive.

In various aspects, the invention includes methods of identifying parathyroid hormone receptors ligands, and osteoregenerative agents, methods of diagnosing bone disorders, and methods of treating bone diorders. For example, in one aspect, the invention provides a method of identifying a parathyroid hormone receptor ligand by providing a test cell population that includes one or more cells capable of expressing one or more nucleic acids sequences responsive to parathyroid hormone and contacting the test cell population with the test agent. Levels of expression of one or more sequences, termed OST sequences, are then compared to the levels of expression of the corresponding nucleic acids in a reference cell population. The reference cell population contains cells whose parathyroid hormone exposure status is known, i.e., the reference cells are known to have been exposed to parathyroid hormone, or are known not to have been exposed to the hormone. When the former type of reference cell population is used, an similar expression profiles of the OST nucleic acids sequences in the test cell population and the reference cell population indicates that the test agent is a ligand for the parathyroid hormone receptor.

The invention in a further aspect includes a method of selecting an individualized therapeutic agent appropriate for a particular subject. The method includes providing from the subject a test cell population comprising a cell capable of expressing one or more nucleic acids sequences responsive to osteoregenerative agents, contacting the test cell population with the therapeutic agent, and comparing the expression of the nucleic acids sequences in the test cell population to the expression of the nucleic acids sequences in a reference cell population.

In a further aspect, the invention provides a method of diagnosing or determining susceptibility to a bone disorder, e.g., osteopenia, osteopetrosis, or osteoporosis. The method includes providing from the subject a cell population comprising a cell capable of expressing one or more osteorengerative genes, and comparing the expression of the nucleic acids sequences to the expression of the nucleic acids sequences in a reference cell population that includes cells from a subject not suffering from a bone disorder.

Also provided are novel nucleic acids whose expression is responsive to the effects of parathyroid hormone, as well as polypeptides encoded by the nucleic acids.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In the case of conflict, the present specification, including definitions, will control. In addition, the materials, methods, and examples are illustrative only and not intended to be limiting.

Other features and advantages of the invention will be apparent from the following detailed description and claims.

### BRIEF DESCRIPTION OF THE DRAWINGS

FIG. 1 shows a statistically relevant gene expression cluster (Cluster 3) identified by the SOM data analysis.

FIG. 2 shows an example of a gene, TIMP-1, conforming to SOM3.

FIG. 3 shows a statistically relevant gene expression cluster (Cluster 7) identified by the SOM data analysis.

FIG. 4 shows an example of a gene, collagenase-3, conforming to SOM7.

FIG. 5 shows a statistically relevant gene expression cluster (Cluster 10) identified by the SOM data analysis.

FIG. 6 shows an example of a gene, growth potentiating factor, conforming to SOM10.

### DETAILED DESCRIPTION OF THE INVENTION

The present invention is based in part on the discovery of changes in expression patterns of multiple nucleic acid sequences in rodent bone marrow cells following exposure parathyroid hormone.

The differentially expressed nucleic acids were identified by administering parathyroid hormone to skeletally mature female rats that were bilaterally ovariectomized to mimic post-menopausal osteoporosis. Four to six weeks post overectomy, the animals were treated with parathyroid hormone by injection once daily for three days. Conrol animals received vehicle alone. At 2 hours, 6 hours and 24 hours post injection, animals were sacrificed and proximal tibiae (including marrow) were harvested. Tibia were flash frozen in liquid nitrogen. One Tibia from each animal were used to harvest RNA, and the remaining tibia were kept in the freezer. RNAs from each time point were pooled to yield sufficient RNA for analysis and then split into 3 equal samples prior to cDNA preparation. Genes whose transcript levels varied relative to the contol samples were identified using GENECALLING™ differential expression analysis as described in U.S. Pat. No. 5,871,697 and in Shimkets et al., Nature Biotechnology 17:798-803 (1999). The contents of these patents and publications are incorporated herein by reference in their entirety.

Approximately 32,000-35,000 gene fragments were initially found to be differentially expressed in rat tibiae, in response to parathyroid hormone treatment. Genes fragments whose expression levels appeared to increase or decrease more than 1.5-fold compared to control tissues were selected for further analysis. Using confirmatory procedures as described in U.S. Pat. Nos. 5,871,697 and 5,972,693, 48 single copy nucleic acid sequences whose expression levels differed in parathyroid hormone treated tissue and tissue treated with vehicle alone were chosen for further characterization. These sequences are referred to herein as OST 1-48 A summary of the OST sequences analyzed is presented in Tables 1 and 2.

Thirty-two sequences (OST: 1-32) represent novel rat genes. The 16 other sequenced identified have been previously described.

For some of the novel sequences (i.e., OST 1-32), a cloned sequence is provided along with one or more additional sequence fragments (e.g., ESTs or contigs) which contain sequences identical to, or substantially identical to, the cloned sequence. Also provided is a consensus sequences which includes a composite sequence assembled from the cloned and additional fragments. In addition, for some of the novel sequences open reading frames (ORFs) were identified, for these sequences the encoded polypeptide sequence is also provided.

For a given OST sequence, its expression can be measured using any of the associated nucleic acid sequence in the methods described herein. For previously described sequences (OST:33-48), database accession numbers are provided. This information allows for one of ordinary skill in the art to deduce information necessary for detecting and measuring expression of the OST nucleic acid sequences.

Parathyroid Hormone Responsive Novel Nucleic Acid Sequences

| | | | Nucleic | Poly- | | | | | | | | | | |
|---|---|---|---|---|---|---|---|---|---|---|---|---|---|---|
| | | OST | Acid | peptide | | | | | | | | | | |
| | GeneBank | Assigne- | SEQ ID | SEQ ID | Parathyroid Hormone Effect on Transcript level* | | | | | | | | | |
| Gene Name | Accessionn No | ment | No: | NO; | 5687 | 5688 | 5689 | 5690 | 5691 | 5692 | 5693 | 5694 | 5695 | 5696 |
| cgrrd0g0143.8 15695- | cgrrd0g0143.8_{—} | 1 | 1 | 50, 51, | 1.9 | | 1.8 | | 2.5 | | 2.2 | 1.7 | 2.1 | 1.9 |
| 182: Novel [N] [CRGN- | 15695-182 | | | 52, 53 | | | | | | | | | | |
| Other] [Client-Other] | | | | | | | | | | | | | | |
| cgrrd0v0398.1 15695- | cgrrd0v0398.1_{—} | 2 | 2 | | | | 1.5 | 1.9 | | | 1.8 | | 1.9 | 2.6 |
| 364: Novel [N] [CRGN- | 15695-364 | | | | | | | | | | | | | |
| Other] [Client-Other] | | | | | | | | | | | | | | |
| cgrrd0y0250.7 15695- | cgrrd0y0250.7_{—} | 3 | 3 | | 1.6 | | 1.6 | | | | 2.5 | 2.4 | 2.1 | 1.9 |
| 128: novel [N] [CRGN- | 15695-128 | | | | | | | | | | | | | |
| Other] [Client-Other] | | | | | | | | | | | | | | |
| cgrrf0i0125.4 15695- | cgrrf0i0125.4_{—} | 4 | 4 | | | | | | | 2.1 | 2.4 | 2.1 | 2.9 | 1.9 |
| 2: [CRGN-Other] | 15695-2 | | | | | | | | | | | | | |
| [Client-Other] | | | | | | | | | | | | | | |
| cgrrg1n0166.8 15694- | cgrrgln0166.8_{—} | 5 | 5 | 33 | | | | | | | | 3.8 | | |
| 4: novel [N] [CRGN- | 15694-4 | | | | | | | | | | | | | |
| Other] [Client-Other] | | | | | | | | | | | | | | |
| BTK: Rat ortholog | cgrrg1n069.8_{—} | 6 | 6 | 34 | | | | | | | | | 2.6 | 2.7 |
| of Bruton | 15695-171 | | | | | | | | | | | | | |
| agammaglobulinemia | | | | | | | | | | | | | | |
| tyrosine kinase (Btk) | | | | | | | | | | | | | | |
| [N] [CRGN-SMT] | | | | | | | | | | | | | | |
| [Client-DM] | | | | | | | | | | | | | | |
| cgrri0r0237.7 5694- | cgrri0r0237.7_{—} | 7 | 7 | 35, 36 | | | | | | | 2.1 | 2.1 | | 2.2 |
| 33: Rat ortholog of | 5694-33 | | | | | | | | | | | | | |
| human protein of | | | | | | | | | | | | | | |
| unknowm function [N] | | | | | | | | | | | | | | |
| [CRGN-Other] [Client- | | | | | | | | | | | | | | |
| Other] | | | | | | | | | | | | | | |
| cgrrl0a0264 15694-5: | cgrrl0a0264_ | 8 | 8 | 37 | | | | | | | | 1.8 | 1.9 | 1.5 |
| Rat ortholog of | 15694-5 | | | | | | | | | | | | | |
| human translation | | | | | | | | | | | | | | |
| initiation factor eIF- | | | | | | | | | | | | | | |
| 3 [N] [CRGN-Other] | | | | | | | | | | | | | | |
| [Client-DM] | | | | | | | | | | | | | | |
| cgrrl0h045.6 15695- | cgrrl0h045.6_{—} | 9 | 9 | 38 | | | | | | 9.8 | | | 8.9 | 8.5 |
| 95: Rat ortholog of | 15695-95 | | | | | | | | | | | | | |
| pseudouridine synthase | | | | | | | | | | | | | | |
| [N] [CRGN-Other] | | | | | | | | | | | | | | |
| [Client-Other] | | | | | | | | | | | | | | |
| cgrrl0h061.1 15696- | cgrrl0h061.1_{—} | 10 | 10 | 39 | | | | | | 1.9 | 2.2 | 2 | | 2.7 |
| 38: novel [N] [CRGN- | 15696-38 | | | | | | | | | | | | | |
| Other] [Client-Other] | | | | | | | | | | | | | | |
| cgrrl0k069.1 15695- | cgrrl0k069.1_{—} | 11 | 11 | 40 | 2.2 | 2.7 | | | | | 2 | 2 | 2.5 | 1.9 |
| 268: Novel [N] [CRGN- | 15695-268 | | | | | | | | | | | | | |
| Other] [Client-Other] | | | | | | | | | | | | | | |
| cgrrl0n0276.1 15694- | cgrrl0n0276.1_{—} | 12 | 12 | 41 | | | | 2.3 | | 2.1 | 2.2 | 4.4 | 4.2 | 2.8 |
| 115: Novel [N] [CRGN- | 15694-115 | | | | | | | | | | | | | |
| Other] [Client-Other] | | | | | | | | | | | | | | |
| cgrrl0u0131.2 15695- | cgrrl0u0131.2_{—} | 13 | 13 | 42 | | | | | | | | | 2.3 | |
| 277: [N] [CRGN-Other] | 15695-277 | | | | | | | | | | | | | |
| [Client-Other] | | | | | | | | | | | | | | |
| cgrrm0r094.9 15695- | cgrrm0r094.9_{—} | 14 | 14 | 43 | | | | 1.5 | 1.5 | 1.4 | 2.4 | 2.3 | 2.4 | 2.4 |
| 10: [CRGN-AT] | 15695-10 | | | | | | | | | | | | | |
| [Client-DM] | | | | | | | | | | | | | | |
| cgrrm1n0153.3 15695- | cgrrm1n0153.3_{—} | 15 | 15 | 44 | | | | | | | 1.8 | 1.8 | 2.2 | 2.1 |
| 97: [CRGN-Other] | 15695-97 | | | | | | | | | | | | | |
| [Client-Other] | | | | | | | | | | | | | | |
| cgrrm1n0191.4 15694- | cgrrm1n0191.4_{—} | 16 | 16 | 45 | | | | | | 1.6 | 2.1 | 2.1 | 2.2 | 1.8 |
| 65: novel [N] [CRGN- | 15694-65 | | | | | | | | | | | | | |
| Other] [Client-Other] | | | | | | | | | | | | | | |
| cgrrm1n0251.6 15694- | cgrrm1n0251.6_{—} | 17 | 17 | 46 | | | | | | | 2 | 2.2 | 2.2 | |
| 131: novel [N] [CRGN- | 15694-131 | | | | | | | | | | | | | |
| Other] [Client-Other] | | | | | | | | | | | | | | |
| cgrrm1n060.1 15694-1: | cgrrm1n060.1_{—} | 18 | 18 | | | | | | | | 2.1 | 2 | 2 | 2.2 |
| Novel [N] [CRGN- | 15694-1 | | | | | | | | | | | | | |
| Other] [Client-Other] | | | | | | | | | | | | | | |
| cgrrm1y066.8 15694- | cgrrm1y066.8_{—} | 19 | 19 | 47 | | | | | | | 2.6 | 2.6 | 2.3 | |
| 184: [CRGN-Other] | 15694-184 | | | | | | | | | | | | | |
| [Client-DM] | | | | | | | | | | | | | | |
| cgrrp0t069.6 15695- | cgrrp0t069.6_{—} | 20 | 20 | | | | 2.3 | 5.2 | 3.9 | 5.5 | 7.4 | | 4.4 | 2.9 |
| 213: Novel [N] [CRGN- | 15695-213 | | | | | | | | | | | | | |
| Other] [Client-Other] | | | | | | | | | | | | | | |
| cgrru0f0141.9 15691- | cgrru0f0141.9_{—} | 21 | 21 | | | 2.2 | | | 2.1 | | | 2.3 | | |
| 117: novel [N] [CRGN- | 15691-117 | | | | | | | | | | | | | |
| AT] [Client-DM] | | | | | | | | | | | | | | |
| cgrrw0c0154.8 15695- | cgrrw0c0154.8_{—} | 22 | 22 | | | | | | | 1.7 | 1.4 | 1.5 | 2 | 1.6 |
| 11: [N] [CRGN-Other] | 15695-11 | | | | | | | | | | | | | |
| [Client-DM] | | | | | | | | | | | | | | |
| cgrry0n0436.5 5695- | cgrry0n0436.5_{—} | 23 | 23 | | | | | | | | | | 2.5 | |
| 364: [CRGN-AT] | 5695-364 | | | | | | | | | | | | | |
| [Client-DM] | | | | | | | | | | | | | | |
| gber ai179093: | gber_ai179093 | 24 | 24 | | | 4.7 | | | 4.5 | | | 4.2 | 2.6 | 2.5 |
| EST222777 Normalized | | | | | | | | | | | | | | |
| rat spleen, Bento | | | | | | | | | | | | | | |
| Soares Rattus sp. cDNA | | | | | | | | | | | | | | |
| clone [N] [CRGN-SMT] | | | | | | | | | | | | | | |
| [Client-DM] | | | | | | | | | | | | | | |
| gber ai179507: | gber_ai179507 | 25 | 25 | | | 3.3 | | 2.7 | 3.6 | 2.5 | | 2.7 | | |
| EST223226 Normalized | | | | | | | | | | | | | | |
| rat spleen, Bento | | | | | | | | | | | | | | |
| Soares Rattus sp. cDNA | | | | | | | | | | | | | | |
| clone [N] [CRGN-PT] | | | | | | | | | | | | | | |
| [Client-DM] | | | | | | | | | | | | | | |
| gber ai237030: | gber_ai237030 | 26 | 26 | | | 2.7 | | | 3.1 | | | 2.9 | 1.5 | |
| EST233592 Normalized | | | | | | | | | | | | | | |
| rat ovary, Bento | | | | | | | | | | | | | | |
| Soares Rattus sp. cDNA | | | | | | | | | | | | | | |
| clone [N] [CRGN-Other] | | | | | | | | | | | | | | |
| [Client-Other] | | | | | | | | | | | | | | |
| gber ai599398: | gber_ai599398 | 27 | 27 | 48 | 4.4 | 3.7 | | 1.4 | | 1.6 | 1.7 | 1.6 | 1.8 | 2 |
| EST251101 Normalized | | | | | | | | | | | | | | |
| rat embryo, Bento | | | | | | | | | | | | | | |
| Soares Rattus sp. cDNA | | | | | | | | | | | | | | |
| clone [CRGN-Other] | | | | | | | | | | | | | | |
| [Client-DM] | | | | | | | | | | | | | | |
| gber ai713362: UI- | gber_ai713362 | 28 | 28 | | | 2.2 | | | 3 | | | 2.3 | | |
| R-AD1-zs-d-02-0-UI.s1 | | | | | | | | | | | | | | |
| UI-R-AD1 Rattus | | | | | | | | | | | | | | |
| norvegicus cDNA clone | | | | | | | | | | | | | | |
| [N] | | | | | | | | | | | | | | |
| gber aw142014: | gber_aw142014 | 29 | 29 | | | 3.2 | | | 3.3 | | | 3.8 | | |
| EST292129 Normalized | | | | | | | | | | | | | | |
| rat ovary, Bento | | | | | | | | | | | | | | |
| Soares Rattus sp. cDNA | | | | | | | | | | | | | | |
| clone [N] [CRGN-Other] | | | | | | | | | | | | | | |
| [Client-Other] | | | | | | | | | | | | | | |
| gber h34286: | gber_h34286 | 30 | 30 | | | | | 2.1 | | | | | | |
| EST111063 Rat PC-12 | | | | | | | | | | | | | | |
| cells, NGF-treated (9 | | | | | | | | | | | | | | |
| days) Rattus sp. cDNA | | | | | | | | | | | | | | |
| 5″ [N] [CRGN-SMT] | | | | | | | | | | | | | | |
| [Client-DM] | | | | | | | | | | | | | | |
| pg rn gbem aw260033: | pg_rn_gbem_{—} | 31 | 31 | 49 | | | | | | | 2 | | 3 | 2.5 |
| Norway rat similar | aw260033 | | | | | | | | | | | | | |
| to mouse gbem_aw260033 | | | | | | | | | | | | | | |
| um82d07.y1 Sugano | | | | | | | | | | | | | | |
| mouse liver mlia Mus | | | | | | | | | | | | | | |
| musculus cDNA clone | | | | | | | | | | | | | | |
| [N] [CRGN-PT] [Client- | | | | | | | | | | | | | | |
| DM] | | | | | | | | | | | | | | |
| scr sc-2228096 1: | scr_sc- | 32 | 32 | | | 2.4 | | 1.5 | 2.1 | 2.4 | | 3.2 | | |
| Based on x95087, | 2228096_1 | | | | | | | | | | | | | |
| R. norvegicus mRNA for | | | | | | | | | | | | | | |
| parathyroid hormone | | | | | | | | | | | | | | |
| regulated sequence | | | | | | | | | | | | | | |
| (92 bp), 253 bp. [CRGN- | | | | | | | | | | | | | | |
| AT] [Client-DM] | | | | | | | | | | | | | | |

TABLE 2
Parathyroid Hormone Responsive Nucleic Acid Sequences

| | | OST | Parathyroid Hormone Effect on Transcript level* | | | | | | | | | |
|---|---|---|---|---|---|---|---|---|---|---|---|---|
| Gene Name | GenBank No | Assignement | 5687 | 5688 | 5689 | 5690 | 5691 | 5692 | 5693 | 5694 | 5695 | 5696 |
| BSP*: Rat | j04215 | 33 | 1.7 | 1.4 | 1.5 | 2 | 1.9 | 1.5 | 2.1 | 1.9 | 2.2 | 2.3 |
| cell-binding bone | | | | | | | | | | | | |
| sialoprotein mRNA | | | | | | | | | | | | |
| [CRGN-PT] [Client- | | | | | | | | | | | | |
| DM] | | | | | | | | | | | | |
| lumican: Rat | x84039 | 34 | | | | | | | 1.6 | | 1.7 | 2 |
| mRNA for lumican. | | | | | | | | | | | | |
| [CRGN-AT] [Client- | | | | | | | | | | | | |
| DM] | | | | | | | | | | | | |
| RoBo-1: Rat | af04l083 | 35 | 1.5 | 1.8 | 1.5 | 1.7 | 1.8 | 1.7 | 1.6 | 1.4 | 1.9 | 1.8 |
| RoBo-1 mRNA [CRGN- | | | | | | | | | | | | |
| At] [Client-DM] | | | | | | | | | | | | |
| osteonectin: | y13714 | 36 | | | 11.7 | 1.5 | 8.7 | | 1.6 | 1.5 | 1.8 | 2.5 |
| Rat osteonectin | | | | | | | | | | | | |
| mRNA. [CRGN-AT] | | | | | | | | | | | | |
| [Client-DM] | | | | | | | | | | | | |
| G-protein alpha: | af107844 | 37 | | | 1.5 | | 1.7 | 1.7 | | | | 2.2 |
| Rat G(s) alpha | | | | | | | | | | | | |
| [CRGN-Other] | | | | | | | | | | | | |
| [Client-Other] | | | | | | | | | | | | |
| G-protein alpha: | m12672 | 38 | | 3.1 | | | | 3.1 | | | | |
| Rat guanine | | | | | | | | | | | | |
| nucleotide-binding | | | | | | | | | | | | |
| protein G-1, alpha | | | | | | | | | | | | |
| subunit mRNA, | | | | | | | | | | | | |
| [CRGN-Other] | | | | | | | | | | | | |
| [Client-Other] | | | | | | | | | | | | |
| PTP: Rat | m33962 | 39 | | 2.5 | | | 2.3 | | | 2.4 | | 1.5 |
| protein-tyrosine- | | | | | | | | | | | | |
| phospatase mRNA | | | | | | | | | | | | |
| [CRGN-SMT] | | | | | | | | | | | | |
| [Client-DM] | | | | | | | | | | | | |
| collagenase: | m60616 | 40 | 3.6 | 2.9 | 1.7 | 1.6 | 2.3 | 1.5 | 1.7 | 2.6 | 1.9 | |
| Rat collagenase | | | | | | | | | | | | |
| mRNA [CRGN-SMT] | | | | | | | | | | | | |
| [Client-DM] | | | | | | | | | | | | |
| TgaseII*: Rat | af106325 | 41 | 1.4 | 4.3 | 3.1 | 1.4 | 5.4 | 2.9 | | 6.9 | 4.2 | |
| tissue-type | | | | | | | | | | | | |
| transglutaminase. | | | | | | | | | | | | |
| [CRGN-AT] [Client- | | | | | | | | | | | | |
| DM] | | | | | | | | | | | | |
| TIMP1*: Rat | u06179 | 42 | 1.9 | 5.1 | 2.7 | | 10.6 | 5.1 | 2.9 | 8.6 | 4 | |
| tissue inhibitor | | | | | | | | | | | | |
| of | | | | | | | | | | | | |
| metalloproteinase- | | | | | | | | | | | | |
| 1 mRNA [CRGN-PT] | | | | | | | | | | | | |
| [Client-DM] | | | | | | | | | | | | |
| Cathepsin E: | d45187 | 43 | 1.8 | | | | 1.5 | 1.5 | 1.9 | 1.9 | 3.5 | 2.3 |
| Rat cathepsin E | | | | | | | | | | | | |
| [CRGN-SMT] | | | | | | | | | | | | |
| [Client-DM] | | | | | | | | | | | | |
| carbonic | x58294 | 44 | | | | | | | | 2.4 | 2.7 | 2 |
| anhydrase II: Rat | | | | | | | | | | | | |
| carbonic anhydrase | | | | | | | | | | | | |
| II mRNA [CRGN-SMT] | | | | | | | | | | | | |
| beta-tubulin: | ab011679 | 45 | | 2 | 1.8 | 1.6 | 1.7 | 1.7 | 1.5 | | 2.1 | 2.3 |
| Rat class I beta- | | | | | | | | | | | | |
| tubulin mRNA. | | | | | | | | | | | | |
| [CRGN-Other] | | | | | | | | | | | | |
| [Client-DM] | | | | | | | | | | | | |
| IL-4 receptor: | ab015746 | 46 | | 2.6 | 1.3 | | 2.2 | | 2 | 2.5 | | 1.4 |
| Rat interleukin-4 | | | | | | | | | | | | |
| receptor mRNA | | | | | | | | | | | | |
| (soluble form) | | | | | | | | | | | | |
| [CRGN-AT] [Client- | | | | | | | | | | | | |
| DM] | | | | | | | | | | | | |
| CKB: Rat brain | m14400 | 47 | 3 | 2.8 | | 1.6 | 1.8 | 1.6 | | 7.2 | 2.6 | 2.5 |
| creatine kinase | | | | | | | | | | | | |
| [CRGN-SMT] | | | | | | | | | | | | |
| [Client-DM] | | | | | | | | | | | | |
| MAC-1 | | 48 | | | | .2 | | | 4.1 | 4 | 4.7 | 5.−8 |

*Treatment Groups
Job 5687. Day 0 Control vs. Day One Vehicle, 2 hrs.
Job 5688: Day One PTH, 2 hrs. vs. Day One Vehicle, 2 hrs.
Job 5689: Day One PTH, 6 hrs. vs. Day One Vehicle, 2 hrs.
Job 5690: Day One PTH, 24 hrs. vs. Day One Vehicle, 2 hrs.
Job 5691: Day Two PTH, 2 hrs. vs. Day One Vehicle, 2 hrs.
Job 5692: Day Two PTH, 6 hrs. vs. Day One Vehicle, 2 hrs.
Job 5693: Day Two PTH, 24 hrs. vs. Day One Vehicle, 2 hrs.
Job 5694: Day Three PTH, 2 hrs. vs. Day One Vehicle, 2 hrs.
Job 5695: Day Three PTH, 6 hrs. vs. Day One Vehicle, 2 hrs.
Job 5696: Day Three PTH, 24 hrs. vs. Day One Vehicle, 2 hrs.

Below follows additional discussion of the novel nucleic acid sequences whose expression is differentially regulated in the presence of parathyroid hormone.

OST1 (d0g0-143.8)

OST1 is a novel 144 bp gene fragment. The nucleic acid has the following sequence:

| | |
|---|---|
| NAATTCCAGCACACAAAGAGCTGAGAAGAACCAGAAGCC | (SEQ ID:1) |
| CTAACATGGAGGCCGGGGAGGAGGCTGAGGCACCATCAA | |
| GAAGAGGCTTCTCATCATCTTCATCCCCACGCTCATTCT | |
| CAGAGCTCTCCTTTGCCGAGAGGATCC. | |

Analysis revealed four potential open reading frames. Frame: +1 includes nucleotide 25 to 144, encoding the 40 amino acid polypetide sequence: EEPEALTWRPGRRLRHHQEEASHHLHPHAHSQSSPLPRGS (SEQ ID NO:50) Frame: +3 includes nucleotide 3 to 143, encoding the 47 amino acid polypeptide sequence: IPAHKELRRTRSPNMEAGEEAEAPSRRGFSSSSSPRSFSELSFAERI (SEQ ID NO:51) Frame: −1 includes nucleotide 1 to 102 encoding the 34 amino acid polypeptide sequence: GSSRQRRALRMSVGMKMMRSLFLMVPQPPPRPPC (SEQ ID NO:52) Frame: −3 includes nucleotides 3 to 143-47, encoding amino acid polpeptide sequence ILSAKESSENERGDEDDEKPLLDGASASSPASMLGLLVLLSSLCAGI (SEQ ID NO:53)

OST2 (d0v0398.1)

OST2 is a novel 1002 bp gene fragment. The nucleic acid has the following sequence:

| 1 | CGGCCGGAAAGGGGGCCGCCCTCTCGCCCGTTACGC | (SEQ ID:2) |
|---|---|---|
| | TTAACGCACGTTCTTGTTTTTTTTTTTTTTCCCCTG | |
| GGGCTGGG | | |
| 81 | GATCGAACCCAGGGCCTTGCGCTCTACCGCTGAGCT | |
| | AAATCCCCAACCCCTGCATTAGAATTCTTATTCACA | |
| CAGTAAGA | | |
| 161 | CAGAATTTGAATTAGAGACCCAGAAGCCTCTGGCAA | |
| | CCCCAAGTCAAACAAGAGCCCACAGTGTTGGCATTG | |
| ACAAGCCT | | |
| 241 | CTCCCCAGTTATACTTCATGGAAATCAGCTCTCCAG | |
| | CACTGCCCACACCCACAGGCCGGCCTGTGACACGGT | |
| GTTGACAC | | |
| 321 | CATCTATTGAGCAACTAAGAAAAGCTATTCAAACCC | |
| | TTCCCCATAAAGAGTGAAGCAAAACAATTTTGCTTT | |
| TCCCCTTG | | |
| 401 | TAGGATTTTGGTATTTTCCAGGCAACTGTCTGTGCA | |
| | TCTCAACCTTCAGTCACCGCATGAAGGATTTTCATG | |
| GGTCTTTA | | |
| 481 | GATTTTCATCTGAGTTATTTAGTTCCCAAGCTATTA | |
| | TTCCCACCAACCCACATTTACAAGCACATCCCTTAC | |
| TGGCACCA | | |
| 561 | ACTCCACCATCATCAATCCTCTGGACAATACACCAT | |
| | TGCCAAGCTGAGCCCAGCTCTCCCCCACACGTCCCA | |
| GTCCCACA | | |
| 641 | TTTGGTGCCAAAAGTACCACCAAGCTTGACCCAGTA | |
| | AAACTCAAATTTCACCTCGGAAGGCCATGTGCACAG | |
| TCAGACCT | | |
| 721 | GTCATTCAGCAGAGCAGCGCTTGCCCATCACCCAGT | |
| | ACCAAACCCACGGACCACCGCAGGCCACTGTTCCCT | |
| TAATACAA | | |
| 801 | AGCCATCCCAAGTTAGATATGCCATCTACAAACTAC | |
| | AGAATTCGCGGCTAACTCGTGCCTTCCCACATTTAA | |
| AAACACAA | | |
| 881 | CCCCCCAACCCATTTAACAACAGGCTTCATCTAAGA | |
| | CTCTTCCACACCAGTCATTTAAAACCCAACAGTTTT | |
| CCCCAACA | | |
| 961 | CCCAAAAGTTCTGTTAAGAATTCCCCTTCTCTTACC | |

TTTGG

OST3 (d0y0-250.7)

OST3 is a novel 700 bp gene fragment. The nucleic acid has the following sequence:

| | |
|---|---|
| TTTTTTTTTTTTTTTTTTTTACCAGGCCCTTCAAATTTT | (SEQ ID:3) |
| TACTAAGACTGTGCGTTCCAACCATGAAATGTAGGGAGT | |
| CAAGAGCTATCTCACTGAGGACAGGGTTTGTTTGGATGC | |
| TGGGTTCCTCACAAGATGGGTGATATGTTTAACAGTGGA | |
| GTTCTGTAAAGTCACCAGATGTAACTGTAAACCACACTG | |
| TGTCACAAAAGGCTCACAGCACAGCATGTGTGGGCACTC | |
| AGGGTCAGTCGGGGTGAGAAAGGGCCAGCTCCTGTGTGG | |
| TGTGGCTGTTAGAGCAACCTGTTGACCTGGGGGCAGAAG | |
| TGACCAGGGCAGAATGAAAGCGTACAGACTGGAGGATAA | |
| GGCTAGTGCTGTCTTGAGGGACCAGGACCCAAGCTCTCC | |
| CTCAGCTGTAGACTAGTTTGGTGAAGCTGGTGTCAGCGA | |
| ATGACATGGATGTAATCGCATAGACCAGCCACTGCCTGG | |
| GCCAGCAACTACAGGTCCCAAGACAGGCCTGAGGACCTC | |
| AGCTCCCGATGCTCCTGCAGCCGAGAAGAGAGCACACAG | |
| CCCCACAAGGCTGCGAGGCTGTCAGGAGCACAGAGCAGG | |
| CATCTGGTGCAGAACTTTATGAGCTATGGATGTGAGTGC | |
| GGAATATGCGGTGCTTTTGGGAAATTTAAGGATGAAGTC | |
| ACGCCCCTCCTCCAAGCACCTGGTAAGCTGGGGATCC. | |

OST4 (f0i0125.4)

OST4 is a novel 125 bp gene fragment. The nucleic acid has the following sequence:

| | |
|---|---|
| AGATCTGGACAAGTTACTGCTGAGTTAGCACAGACAGTA | (SEQ ID:4) |
| GAGGAACTGTGTATTCTTAGCCATGGAAACTATTGACTG | |
| ACATTTTTCTGTTAGAAGATAAAATGAAAATTTACCTAA | |
| ACCCTAGG. | |

OST5 (g1n0-166.8)

OST5 is a novel 550 bp gene that is represented by the following nucleotide sequence:

| | |
|---|---|
| TTTTTTTTTTTTTTTTTCTGAAGCAAAGTCTGACTTTAT | (SEQ ID:5) |
| TTGCCTGGACACATGTCTGACCCCCACTTGGGGTGCTCA | |
| GGGGAGCTCTGGCCATGAGGTGCCGGCTCACACATGCTG | |
| CCTTCGATGTCTCAGCACCTCCGTGGGAGTGAAGAGGAA | |
| GTCCTGCCCGCACACCTGACAGTGGTAGGGCCTCTGCAG | |
| GGCTGATGTCTTCTGCGTTGGTACAGGAGCAGCCTCCTC | |
| AGACCCTGGACCATCACTCGGCGCCTCAGGGCAGGTGTT | |
| CTCATGAGCAATGCGCTCCAGAGGTGTAAAGGGCATGTG | |
| CAGGCCACAGTGTGGGCAAGTCCAGAAGGAGCGCAGACA | |
| GTCACTGTACAGGTCAGTGTCACTCGTGAGGCAGTTGTA | |
| GGTGAGATAATCAGAGACAGCGTAGCCCCCCTTGGTGGG | |
| GTGTGGGGACAAGATAGAATTTCCAAAGAGACCGGGGAG | |
| ACTAGGGGCGGTGTGATGGTCTGAGGTCCCACATAGAGG | |
| TTCTGGGCTTCTAGACCAGTGAGCCTTCGGAGGCGGCCT | |
| CGTG. | |

The open reading frame of OST5 encodes a 147 amino acid polypeptide shown below:

| | |
|---|---|
| RGRLRRLTGLEAQNLYVGPQTITTAPSLPGLFGNSILSP | (SEQ ID:33) |
| HPTKGGYAVSDYLTYNCLTSDTDLYSDCLRSFWTCPHCG | |
| LHMPFTPLERIAHENTCPEAPSDGPGSEEAAPVPTQKTS | |
| ALQRPYHCQVCGQDFLFTPTEVLRHRRQHV. | |

OST6 (g1n069.8)

OST6 is a novel 774 bp gene that is represented by the following nucleotide sequence:

| | |
|---|---|
| TTTTTTTTTTTTTTTCTCTTTCCAGTAATTTTATTGTAT | (SEQ ID:6) |
| CAAAACAATCCCTCCCATTTTAATGGCACCATTCTTACC | |
| CCCAAACTCTTAACACTTAACCAACAAGCTGTTGAACAA | |
| CTAGTGGGGCTATTTACATCTTCCCTCCCCTGAAATGTT | |
| CCCATCCCAAATTCAGTCTGTTAGGAGTCTTGAAGTAAT | |
| ACCAGGCCCAGAAATTCCTTGGCCTTTGCTCAGAAGCAC | |
| TATGCTGGGGAGTTAGAGAAGAAAGGCATTCCTAGATGT | |
| ACAGTAGGAGCGTGGAAGGCTAGAGCCCTTGAACCAGGG | |
| AGTTCCTCAGGAAAAAATGGAATTGGGGCTTGTAGAAGA | |
| GAGGAGTAGATCCATGAAGCTTATCAGCCAGCTCAAGAT | |
| TCTTCATCCATGACATCTAGAATGTTACTCAAGAGAATT | |
| TTGAAACTAGGACGTTCATCTGCTTTCTCATGCCAGCAG | |
| CTGTACATGATGGTATATACCCTGTCTGATGCCAAATGA | |
| GGCCTGTAGAGACGTAAGCCTTGAGCAATGTGTTCTGCT | |
| GTCTCACTGTTAGTAAATCTCCCTCGTGGTATTTTCCCC | |
| AGGGAGTAAATTTCCCACATTAGAACCCCAAAAGCCCAG | |
| ATGTCAGATTTGCTGCTGAATTTGCTATACATGAGCACT | |
| TCTGGTGGAGACCACCGGACTGGAAATTTGGAGCCTACC | |
| GAGCTGGTGTATTCATCATCAAGAACATACCTAGACAGG | |
| CCAAAGTCAGATACTTTCACAACTCCTTGATCA. | |

The open reading frame of OST6 encodes a 129 amino acid polypeptide shown below:

| | |
|---|---|
| DQGVVKVSDFGLSRYVLDDEYTSSVGSKFPVRWSPPEVL | (SEQ ID:34) |
| MYSKFSSKSDIWAFGVLMWEIYSLGKIPRGRFTNSETAE | |
| HIAQGLRLYRPHLASDRVYTIMYSCWHEKADERPSFKIL | |
| LSNILDVMDEES. | |

OST7 (i0r0-237.7)

OST7 is a novel 522 bp gene that is represented by the following nucleotide sequence:

| | |
|---|---|
| CGAATTCGAAGTGGAGGCAGCAGGCCACGGACCACCTCC | (SEQ ID:7) |
| AGCGGGAGGGGCAAGGGCTCGGGCTCAGGCAAGGGCATG | |
| GCTAGGGCCAGCGGGAGGCTGGGACCAATGACTGCAGGC | |
| CCGGCTGGGGCTCCTCCTGCCCCCACAGCCACTGCTGCA | |
| CTAGCTTCCTCCAGCCCAGCCGCTGCAGCCACTACTGCT | |
| TCTTCCTTCTCCTTCAGGCCTAAACCAAGGCCTAGGCCC | |
| AGATCTCGAGGAGCTGCTGGCTCTTCCAGGGGAGGTCTC | |
| AGGGCAGCCATAGAACCCAGGGGTGGTCCAGGTGCCCGA | |
| GCCATTGGTGGTAGCATCATGGGTGGTCCAGGAGGCCCA | |
| GGCAGAGGGGGTCCAACCAGGGCCTGGTGTGGAGGCCTC | |
| AGGGCATAGGTCGGGGGCCGCCAGCTGCTCGCCTCAGGA | |
| ACATGGCCTCTCGAGCCTCAGGACTGTCCAGGTGACTTC | |
| GATCAGCAGGGCCAAAGCCAACTGGGCCCACAAAAGGCG | |
| GGCCTCCCACCATGG. | |

The open reading frames of OST7 encode a 148 amino acid polypeptide, and a 43 amino acid polypeptide, shown below:

Frame −2

| | |
|---|---|
| GSRGHVPEASSWRPPTYALRPPHQALVGPPLPGPPGPPM | (SEQ ID:35) |
| MLPPMARAPGPPLGSMAALRPPLEEPAAPRDLGLGLGLG | |
| LKEKEEAVVAAAAGLEEASAAVAVGAGGAPAGPAVIGPS | |
| LPLALAMPLPEPEPLPLPLEVVRGLLPPLRI. | |

Frame −3

MVGGPPFVGPVGFGPADRSHLDSPEAREAMFLRRAAGGPRPMP (SEQ ID: 36).

OST8 (l0a0-264)

OST8 is a novel 1727 bp gene that is represented by the following nucleotide sequence:

| | |
|---|---|
| CCTTCCCTACACCTCACCTATAAATCATGTTCTCATGACATTTTACTGCTAATGTCAAAGAGTAGCAGCTCTTTGAAG | (SEQ ID:8) |
| TGAGGTTGTGTCCACTTCGGTTTCCCAGTCCGACGTGTAAACGTCGGCCAGTGATTGTATCCGCTCATAATAGGCAAA | |
| ATGGGCACAGGCACCTCTGTTTTTTTTTTTTTTTTTTTACATTCTCAACAGACCTTTAATGACCGGGGTGAGTTTAGG | |
| AACAAAAAGGTAGATAGTCTGGTCATAGGGACGACAAGAAGTGAGGACCTCAGTAAGCTGTCTGAGACTGCTGCTGGC | |
| GGTAGCCACCACGGCGCATATAGCCTTCATTTTTTCGGTAGCCATCCTTTTGGTCTCGGAAATAGCCGCCATAGGTTC | |
| CTTGCTTATGGTCAAACACACGTTCATTATTCTCCACTAGGCTGCCAAGCTTCTCAGCCAGCTGCAGAGCCAAGTTCT | |
| GTTGGGCAGTGGGCTCAGTACGGTGCATCACTACCGTCTGCGTTGGCTGGTCCAGGGAAGCCATCAATTCTTCATTAA | |
| TGATCATCTTGCTGATGATGGAGTGAACAGTGGGCAGATCCAGCTCAAACATATCTGAGAGTGTCTCCATACTGATTG | |
| AGTCATAGACACTGCTATAGGTAAAAAGGTAGGTCCTCAGAGACTCTTCCTGGATCTTCCGAACTAGCATGGTGCGAA | |
| CTTTGTCAGCCTCAGGGAAAAGGTCCCACACTTTCCCATTCATCTTTTCATTAATGATGAAACTGTGGCAGGTCTTCC | |
| AGTCACCCATCTTCATGGCCTTGGAGGCAGCAACCACATGCTCCCTCATTGACTCAGGAGGACCTAGCAGGGGCTGCC | |
| GCTCGCCCACCCGCAGTTGGTGATGGAACTGCTTGCTGATCATGCGTCGGCGGGCATCACTCTCATGGGCAGCCATGT | |
| AGGGGATCTCCAGGAGCATAGCAGACACCAGATACACACACTCCNAGCAGTTCCAGGTTGATGTGCAGCTGAAAGGGC | |
| ACCTGCCGGCGTCGCTCTACCTTCTCCTGTTCCTGGTTGCGCTCCTAAAGGCTGCGCAGCAGCAGACCCTGGCCCAGA | |
| AGCTCCTTGGCACGACCACTTGACTGAATATCCAGAAGTGCATTGTGTGCATCCTTTGTCAGGCCTTGGCGGAAAGCA | |
| CAGATGCCCAGTTGCACCATGGTACGGTTATACAGGATCTGTACTGGCGGGTCTGCGTGCTGGATGTTGTCTTGTAGG | |
| TGGCTCATGAGCATGAGGTCACGGGCCTGATACCAGCGGGAGTGGAGCGCATGATGGTAGATATGGCAGAGGATGGCA | |
| CAGGTACGGATGCGGTCTGTACGGTCCTTGGCATAGATGTATTTGCACAGTCTTTCCATTAGCACAGCTGAGTCCTCA | |
| CCCTCATTTTCTGCCTGGTCTTGCTCAGACTTTGAGGACCCTTCCGGAGGAGTAAGCTGACGCTGATGGGCCTTGTAA | |
| TCAAACTTGTAGTAAGTGTGCAGGATGCGCCTCAAGTAGATCTGGCAGATCTCCTCAGTGGTACCTTTCTCCTCCAGG | |
| TAGCGCTGTACACGCTCAATGATGGCACATACTTGTGCCTCATCCTTCAGATGTTCCACATACTCTTGGGAGTGAGGA | |
| TCAGTATTTTGCATTATTTTGGTAAATTCTTCATCCATTCGCTCCACCAGAGTTAGGATGCAGCCTCGTACACGCAGT | |
| GGCTGATCAAA. | |

The open reading frame of OST8 encodes a 268 amino acid polypeptide shown below:

| | |
|---|---|
| FDQPLRVRGCILTLVERMDEEFTKIMQNTDPHSQEYVEHLKDEAQVCAIIERVQRYLEEKGTTEEICQIYLRRILHTY | (SEQ ID:37) |
| YKFDYKAHQRQLTPPEGSSKSEQDQAENEGEDSAVLMERLCKYIYAKDRTDRIRTCAILCHIYHHALHSRWYQARDLM | |
| LMSHLQDNIQHADPPVQILYNRTMVQLGICAFRQGLTKDAHNALLDIQSSGRAKELLGQGLLLRSLQERNQEQEKVER | |
| RRQVPFQLHINLELLGVCVSGVCYAPGDPLHGCP. | |

OST9 (l0h0-45.6)

OST9 is novel 249 bp gene that is represented by the following nucleotide sequence:

| | |
|---|---|
| TGTACAGGATGAGAGCTATCGCCTAAGTGCAGAGACCCTGCAGCAGGTCAACCGGCTTCTGGGCTGCTACAAAGGCAC | (SEQ ID:9) |
| GCATAACTTCCATAACTTCACATCACAGAAGGGGCCGAGAGAGCCTAGTGCACGCCGCTACATTCTGGAGATGTACTG | |
| CGAGGAGCCCTTTGTTCGGGAGGQCCTGGAGTTTGCTGTGATCAAGGTGAAGGGCCAGAGCTTCATGATGCACCAGAT | |
| CCGGAAGATGGTTGN. | |

The open reading frame of OST9 encodes a 82 amino acid polypeptide shown below:

| | |
|---|---|
| VQDESYRLSAETLQQVNRLLGCYKGTHNFHNFTSQKGPREPSARRYILEMYCEEPFVREGLEFAVIKVKGQSFMMHQI | (SEQ ID:38) |
| RKMV. | |

OST10 (10h0-61.1)

OST10 is a novel 714 bp gene that is represented by the following nucleotide sequence:

| | |
|---|---|
| TTTTTTTTTTTTTTTTTTCGTTAGAAAGCACTATTTATTTCTTCTTACTCTTGTTTCGTTTCTCTTCTTTCAGCTTCT | (SEQ ID:10) |
| TTTTGGAGACTCTGGGTGTGCTGGCCTTTTTGTATAGGTGATACCCAATCAGGCCCAGGAGTGCTGGCACCATGGCCA | |
| TGCCTACCAGAGGCAAAATGCCCTTCAGCAGCTTTTGCCAGTAGTTGGCTCGAATCAAGGCGATCAGCTCCACATCAT | |
| ACTGCACCACTGCATCGGCTGGGATAGATGGTGGGTACCCTCTCTTTCCATAAGCCAAGTGAGAAGGAATGACTGCTC | |
| TTCGCTTCTCCCCTACACACATGTCCAGAAGGCTCTGCTCCAGACCTGGGATCACCTGCTTTTGGCCAAGTTCTATGA | |
| CCAGAGGATCTCTGGTCAGAGAAGTGTCAATAATGCGTCCATCTGCCAAGCTGCCCGTGTAGTGTATGTGGAGCGTGT | |
| CTCCAAAGGCAGCGGACTCCGTGCACGATTCAGGGGGCTGCACCAGGGTCTCCACTTGGAGGGTCCGGACAGGACTTT | |
| CGGTTTCTACCTCAGCCTCAGCCCGGCACACCGCCCCACTGATCAGCAGCAGCAGCAACAGCCGAAGCGGCAGGAGCA | |
| AAGGGCGCAGGGTCATGATTGGACTGGGTCAGAGCACCGGGGTACCAGGGCAGGCTGTTCGGGTGCACGCGACCAGGA | |
| CCGGTCCTCGTG. | |

The open reading frame of OST10 encodes a 201 amino acid polypeptide shown below:

| | |
|---|---|
| MTLRPLLLPLRLLLLLLISGAVCRAEAEVETESPVRTLQVETLVQPPESCTESAAFGDTLHIHYTGSLADGRIIDTSL | (SEQ ID:39) |
| TRDPLVIELGQKQVIPGLEQSLLDMCVGEKRRAVIPSHLAYGKRGYPPSIPADAVVQYDVELIALIRANYWQKLLKGI | |
| LPLVGMAMVPALLGLIGYHLYKKASTPRVSKKKLKEEKRNKSKKK. | |

OST11 (l0k0-69.1)

OST11 is a novel 1363 bp gene that is represented by the following nucleotide sequence:

| | |
|---|---|
| TTTTTTTTTTTTTTTTTTTTTTTTTAAACAATGACGCCGTTTATTTAAAATGTTTACTCCAAGAAATATAGATATAAA | (SEQ ID:11) |
| AAAAAATTAGACAATAACAGCACTAAACCAGGCACCTTCGACCGAATCCCATCCTCGTCCACTCCCTCTGCGCTACGC | |
| TTTCTCGATGACCAGAAAATTTCAGAGCCCCTGGGAGGCCAGAATGGTTCCTACCCAGGGCTTCCCACCTTGAGTTTC | |
| TGGTGGGAAAGCTCAGGTGAGAATTTTAGCCTGAAGGGAGGGGGGCTGTGGCCAGGCACAGGACTCTCTACCCATAAG | |
| ACACTTTCTGCTCACCCACTGCAGGGCTCCAGCCAAGGGGACTGACTGCTGGCTTTAGGTTTGCTCCCTGGAAGATGA | |
| GCCTAGTTCAGCTCAGGGCTACTGTGTCCTCAATCCTCTTGGTGACCACACGACTAACTCCAGGAGACCCCCAGGACA | |
| GAAGGAGCAGACCCTGAACAGCACACGCCAGACAGAGACCAGGAAAGGCTCCGGTCCCGCACGTGGCAGTGACCGGTG | |
| TCAGTGGCAGGGGTGATGTCTAGGCTTACCTTGGCTATGAGCAAATGTTTTAATCAACGTTGAGGGAAAACACTTTGG | |
| AAAGCATACAGAAAAGGTAGTTAACGTTGAATCGCTTGGAAACGGAACCTCAGGGAGGCTAACCCAATGCACCCTTGG | |
| TCAACTTTTGCTTCCTAAATTCCTTGTCTGACGTCTGGTCCCAGTCCACGTGGGAATGACAGCTGCCACGAGGGGAGT | |
| GCCGGCCTGCTGCTGCTCAGAGCGTGCGTGGGGTGTACTCAGGCAGCCTCTGCAGCCTCTCCTTCTCAGCCTCGCTCT | |
| CATCTCGTGCTATCACCAATGAATGTGAATAGCCCATGGCCACCTGTTCGGAGAAGATGCCATCCAGAGTCTTCACCT | |
| CCTGAGCTGCAGTAGAAGACTTGGGCTTGTGGTCCCCATATCCCAATTCCCCGAAGGTTGGTGATGGGCCCCAGCTGA | |
| TGGTACTCTCGTCGGCCGCTACGATGATGCTGCTCTTCCCGCAGGCAAGGCTCCGGATCCGCCAGCCACAGAGGTCCT | |
| GCACGGCTTTTGGGTACATGGTAGACTCTCGAGAAGTATTGGTAGCCCCCCAGAAAAACAGCCCCCCCACTTCACTGA | |
| CAGCAAAGGAGCAGGTGTAGCCGGCATAGATCTGGGTTGCACCACGTCCCGGGAAATCGAAGAGCTTCACCAGGCGAG | |
| GTACCATCTCATCCTTCTGCTCGGCATGGCCCAGCAGAAGGATGAGATGGTACCTCTGATGTTGGAGCGGCCGCGGCC | |
| TGGGACCCAGGCTGGGGGGAACCTGGGGCATTGTTGT. | |

The open reading frame of OST11 encodes a 188 amino acid polypetide shown below:

| | |
|---|---|
| TTMPQVPPSLGPRPRPLQHQRYHLILLLGHAEQKDEMVPRLVKLFDFPGRGATQIYAGYTCSFAVSEVGGLFFWGATN | (SEQ ID:40) |
| TSRESTMYPKAVQDLCGWRIRSLACGKSSIIVAADESTISWGPSPTFGELGYGDHKPKSSTAAQEVKTLDGIFSEQVA | |
| MGYSHSLVIARDESEAEKERLQRLPEYTPRTL. | |

OST12 (l0n0276.1)

OST 12 is a novel 520 bp gene that is represented by the following nucleotide sequence:

| | |
|---|---|
| TTTTTTTTTTTTTTTTTGAGTTTGAAATTAAACTTTACTCTTTTCAAAGTAATGTAACTTAGAAATGAATAGAGGTTT | (SEQ ID:12) |
| AAATAAACCACACAGAAGTCAGACAGATGTACACGATCTTCTCAAATCCATCAACGATCTTCCCCATCTCCAGATACT | |
| TCGTGTTGAGGTCCTCATATTTGTTTCTCAGCTCCAAGTTCTCCTGCTGCATCACGTTCACCACTGCATCCAGGTCTT | |
| TCTGACTGTATTTCAGCACGTCCACAATAGGCTCGGCAGGAAGGAGACCTCGAGGCTCAAGGACACACAGGGGTGGGG | |
| CCGACACAGGAACATCCAGACCTCCCAAGAAATCTAAGTCGACGAGCTTGGCTTCTGTTGGATTTCCGGAGCCAGACT | |
| CCTCTGTATCCTGTGTACTATTTGTCACTGGGGCCACTGGCACTGGCATTGACCGCAAAGGGCTCTCTTTCAGAAGGG | |
| GGTCAAATTTCAAGTACAAAGACTGCTTCCTCCACGCTGACTCCTTGAACGA | |

The open reading frame of OST12 encodes a 147 amino acid polypeptide shown below:

| | |
|---|---|
| SFKESAWRKQSLYLKFDPLLKESPLRSMPVPVAPVTNSTQDTEESGSGNPTEAKLVDLDFLGGLDVPVSAPPLCVLEP | (SEQ ID:41) |
| RGLLPAEPIVDVLKYSQKDLDAVVNVMQQENLELRNKYEDLNTKYLEMGKIVDGFEKIVYICLTSVWFI. | |

OST13 (l0u0131.2)

OST13 is a novel 984 bp gene that is represented by the following nucleotide sequence:

| | |
|---|---|
| GCTCACTTCACATACTGAAGGACTGATGGCAATCAATGC | (SEQ ID:13) |
| AGAAGCTGTACCGTCCGGCTGCCCCCTTTCCGGAGGCTA | |
| AAGTGCTATGGCTGCCTGAGGGTAAACAAGCCAACAGGG | |
| GTTTTTCCTGGCTTCCTGTCCCAACTTCTCAGGCCCACA | |
| GTCAGCCAGAGAGCCCGGCCTCTTTCCTGACCACCCCCA | |
| TGGCATCTCTATTCAAAAGGCAAAAGGAAAAGATCAAAA | |
| ACTTCAGAATGGGTTAGAGCCCAAGGGCCCCACCCTAAC | |
| CCCACTACAAGCTACTCCAAGTCGGCTACTGGGGAAGCG | |
| AGGGGGAAGCCAGGCCCAGGGCAACCCCCATCTCTGGCA | |
| GTGTCCAGAGGCTCCTGTCTTCAGCGTCACAGCAAAGGC | |
| CTTCTGCCCTTCCCCTGCCAACCTGGCTGGGGGAAGGGG | |
| CTACAACCTTCAGTACTTATTGATTCCAAAGATCCGGAC | |
| TCCATGGAGCTGGGGCAGCTTGGGAATGAGCACACTCAT | |
| CAAGGACACAGTGTTGAGTATGAAGTGGACTTGGTCATA | |
| CTTGGTGTAGAAGCTGGTGAGGAAGTACAGCACAATGGG | |
| CGTGATGGTTCCCGTTGGAAAAAATTTTGTGAACTGGAC | |
| CCCATAGTCCATCTGCTCCCAGTGGGTCAGCAGCCTTGC | |
| CTTGCCCTGGTCCGGAGTCTCGAAGGGTGTCCCTTTCAC | |
| CGTGTGCAGAAAGATGTACATGCCCAGGTTATGGATAAG | |
| GTTGGTGAGGGTCCAGACGACGGGGACGCTCACAAAGGG | |
| GATGCTCAGCAGCACGACGTGCAGGAGTCCGATGGCCAG | |
| CACGTAGGAGAGCCAGATGCCACGGCTGTTCATCACCCG | |
| AGTGTTGGGGTTCACCTCACTGTGTGCTGTGCCCACATT | |
| CATCCTGCCACCCCTCTTGACTGCTGCTCGCGCTGGGCC | |
| GGAGGTCGAGGGCCCGGGAGAGAAGCCCGAGAACCCGGG | |
| CCGCGGCCG. | |

The open reading frame of OST13 encodes a 153 amino acid polypeptide shown below:

| | |
|---|---|
| MNVGTAHSEVNPNTRVMNSRGIWLSYVLAIGLLHVVLLS | (SEQ ID:42) |
| IPFVSVPVVWTLTNLIHNLGMYIFLHTVKGTPFETPDQG | |
| KARLLTHWEQMDYGVQFTKFFPTGTITPIVLYFLTSFYT | |
| KYDQVHFILNTVSLJMSVLIPKLPQLHGVRIFGINKY. | |

OST14 (m0r0-94.4)

OST14 is a novel 919 bp gene that is represented by the following nucleotide sequence:

| | |
|---|---|
| GCAGAGCTACAAATTAACCCNCGACCAAAGGGNAGGAAT | (SEQ ID:14) |
| GATGNGGCAAAGCTAAAATTAACCCTCACTAAAGGGAAT | |
| AAGATTGCGGCCGCACAACTTTTTTTTTTTTTTTTTTTT | |
| TTTTTTTTTTTTTTTTTTTTTTTTTTTTTTGGCAGAGAA | |
| ATGTTTTTTAATTTGGAAAGTAAGGGTTACACTGAGGGG | |
| AGGCAGATTCTGTGTTAACAGACGCGCACTGTGGAGTTG | |
| GTCCAACAGAGCAAAGGGAGTTCTCTGGACGCCCCACCT | |
| CACGTCCAGCAAGAGTCATAGCTGAGCACGAGTCTGAAG | |
| AATCCATGTACGACAAGCTGCCAGCTCAAGAATTCTGAT | |
| CCAGGAGACGTCAAAGAGTGAGCACTCTGCCAGTCTCCA | |
| ACTCCACGGTAAGGAAATGGGAGCGGCTCAGCCGACGAA | |
| GTTCATGACGACGAGGTGCAGGATAGTGCTGGCAAAGAG | |
| AAAATCAGCAAAGGCTCGCTCAGGAGAGATGCCTTGGAA | |
| ATCCGCCTTGTTCTGGGGGTTGATCTGTATTCTCAAGCA | |
| AACCGCTAAGATGAAGCTGCCCACACAAGAGATGAAGCC | |
| AGAGAGGAAAGAGTTGAAGGGGAAGGTGCCCACGAGGAG | |
| ACAGTAGCCGAACTGCAGCGCCCCGGTCAGCAATATATA | |
| AAGGAGATAGGCATCCAGCAACTTCAGCCGCTGTGGAGT | |
| GGAGCTCAAGTACTCCTCCAGGAACCGGGAGATGACGGA | |
| CACCACAGACGCCGACATGACGAACCGGCGAATTCCGAT | |
| TGGCCCGTGCCCAGATCTGGGCTCTATGGGCAATATCCT | |
| AAGATGAGTCTCACTAATCCTAACCAGGCATACCGTCCA | |
| CGGGTGATATGTTACCGCCCCAGTCCAACACATTCCAGC | |
| CTAACAGAGTGTGAGCTGGGGG. | |

The open reading frame of OST14 encodes a 113 amino acid polypeptide shown below:

| | |
|---|---|
| MSASVVSVISRFLEEYLSSTPQRLKLLDAYLLYILLTGA | (SEQ ID:43) |
| LQFGYCLLVGTFPFNSFLSGFISCVGSFILAVCLRIQIN | |
| PQNKADFQGISPERAFADFLFASTILHLVVMNFVG. | |

OST15 (m1n0-153.3)

OST15 is a novel 327 bp gene that is represented by the following nucleotide sequence:

| | |
|---|---|
| TCGGGCTTAGCTCTCCCAGCCTTCTCACCGACCCGGAGC | (SEQ ID:15) |
| GGCCAGAGCTCCGCTTCTCTGGGCGGTAGCCCGAGCTCC | |
| TCCAGAGCCAGAGCGCGGCGCGCCCCACCTGCGGCCTCA | |
| CCATGGTGCTGTTGGCGGCAGCGGTCTGCACGAAAGCGG | |
| GAAAGGCAATTGTTTCTCGACAGTTTGTGGAGATGACCC | |
| GAACTCGGATTGAGGGGTTATTAGCAGCTTTCCCAAAAC | |
| TCATGAACACTGGAAAACAACATACTTTTGTTGAAACAG | |
| AAAGTGTAAGATATGTCTACCAACCTATGGAGAAACTGT | |
| ACATGGTATTGATCA. | |

The open reading frame of OST15 encodes a 69 amino acid polypeptide shown below: MVLLAAAVCTKAGKAIVSRQFVEMTRTRIEGLLAAFPKLMNTGKQHTFVETESVRYVYQPMEKLYMVLI (SEQ ID: 44).

OST16 (m1n0-191.4)

OST16 is a novel 971 bp gene that is represented by the following nucleotide sequence:

| | |
|---|---|
| GAGGAGGTGGCACTGAAACAAACGGGTGACGATGATGAA | (SEQ ID:16) |
| GTGGATGACTTTGAGGACTTCATCTTCAGCCACTTCTTT | |
| GGAGACAAGGCACTGAAGAGGAGGTCAAAGAAGAAGGAG | |
| AAGCAGCCTTGGAACCACAGAGCTGTGGGGCCCAGGGAA | |
| GAGCACAGCCGCCATCCCCACCANTACCACCAAGGCTGA | |
| GCCCTGCTCCTGCTGCAACCACCGGCCTTGGCATGCACC | |
| CAGCTAAAGATCCCTGGAATTATGTGACCCATGCTCACA | |
| CTGTCCAGTGGGTTCCTCCTGGCTCGTTAGTGCCCTTGG | |
| ACGTCCTTCACAGAAGAGAGCAAAGCACTAGGCCCAGCC | |
| CGGGTACCACTTTTGCTTAAAAAAGGCCGTAGTTGACAC | |
| TACTGAACTGTTTAAACAAATGTATGCAAATGATAAAGG | |
| CCCTGGGTACTAAGCATCCTGTAGGAGCCTGCTGGGGAG | |
| GACTCTTATTCTGAAGCCAGAGAGCTGTCCTTTCTTGTG | |
| GCTATCCTGGGTCCACAGCACAGCTTACCACCACCCTGC | |
| ACTGGAGCTACTGGTTGGAGACTTAGCTCCGGACTTGCC | |
| TTGGGGGTTTTTGTTAGCTTTGGAGACAGCTGTCGCTAA | |
| GCCTGAGGAGCCCACACCATGCCTCTTGTGGCTTTCACC | |
| TCCCCACCTGAACTGTACACCATGATAGGGCTCTGTCAC | |
| TTGAGGTGGTCTCTAAGTGCAAGGGGACACCTTGCAGCC | |
| ACCACTCCAGTGTTTGGGCACAATCTAGACACTGCACTC | |
| AACCCTTTTGCCTTTCTGCCCTCAGCCTTCCCGTCTGTT | |
| CCCCATGTTCTTTCCCAAGGAATCTAGCCTTGAGTCCAG | |
| CCACCAATTGTCTCACAATACACAGTGTGGTATTCTTAT | |
| CTTCTGAAAGAGGGCTGAAGGGCTGAGAATGGAGTCAAT | |
| AAAGCAAGGAAGCAAACAAAAAAAAAAAAAAAAAA. | |

The open reading frame of OST16 encodes a 64 amino acid polypeptide shown below: EEVALKQTGDDDEVDDFEDFIFSHFFGDKALKRRSKKKEKQPWNHRAVGPREEHSRHPHXYHQG (SEQ ID: 45).

OST17 (m1n0-251.6)

OST17 is a novel 251 bp gene represented by the following nucleotide sequence:

| | |
|---|---|
| TGTACAAGATGGTGAAGCAGGTGGCATCACACAGCAGAT | (SEQ ID:17) |
| TGGTGCCACAAATGTTCCTCTCGAAGCTATTAATGAACA | |
| AACTAAAATGATTAAAAATTTTGACAGAGAGAATGTCCG | |
| CATTCCAGGGATGCTGATTATTGACACTCCTGGACATGA | |
| GTCTTTCAGTAACCTGAGAAACAGAGGAAGCTCTCTCTG | |
| TGATATTGCCATTTTAGTTGTTGACATTATGCATGGTTT | |
| AGAGCCCCAGACAATTG. | |

The open reading frame of OST17 encodes a 83 amino acid polypeptide shown below: VQDGEAGGITQQIGATNVPLEAINEQTKMIKNFDRENVRIPGMLIIDTPGHESFSNLRNRGSSLCDIAILVVDIMHGL EPQTI (SEQ ID: 46).

OST18 (m1n0-60.1)

OST18 is a novel 1048 bp gene fragment. The nucleic acid has the following sequence:

| | |
|---|---|
| TTTTTTTTTTTTTTTTTTGGGAAGAAAAACCTTTTAATT | (SEQ ID:18) |
| AAAACAATTTACACATTGACACTTATGGCTTAACTAGGA | |
| TAATTTTTGTCCAAGTTAAAAATGTACTAAGGACAAAAC | |
| AGAAGACTGGCGTAAATAATGTACAGCACTTGCTCAAAA | |
| CAGTAGTGAATTGTAAGAGCTATCAAAGCAAAGTCCAAT | |
| TGGAGTAATTAAAGTTTTACATAGGTAAAAATAACTATC | |
| TGAGAAATCAAGAACATATTTAATCGTATCGCACAGAAA | |
| CACTGGTATTAAGTCACCTTCCTACAAAATTTCAAAGCA | |
| TAAGGATAAACTTTTCTACCTACCAGAGACCAACAACTG | |
| ACCTGATTACTCAGAATATCTTTGAAATGTTATGCCTTA | |
| ATTTAACAGAACAATAGTCCATGTTTGTGTATATGGACT | |
| ATCTGGTTACTACCTTGGAACAACTGAAGTTTGTTTTTT | |
| CTGTAATCATCTGATTATGTTTGATAGAGAAACAAAAAT | |
| CCCATTACAATATGGCCATGTCTTCAATTAGGTTAATTT | |
| CCTTATTTATTACTTATAAGCATCATGGATTGATTCTAA | |
| TTAAAAAAGAAACAAAAAAAAACACACCTTAATGAGTAT | |
| AAGATTAAAACAAACTAATTTTAGGTTTGTGTGATTAGT | |
| ACCTTTATAAAGGATTGTAGATGGACTTCCTATATGTAT | |
| ATATAACCAGTCTGTTCTCTCTAGTTCTAACACCACTGA | |
| AATCCCAGTCCTAAAACTAAATTACTTTCAAAAGTAAAC | |
| AAATTTGTACCTTCTATAACAACCCAATAGGCATCTAGC | |
| TGTCATACTACACATTATTACAAAGAGGCACGCTAATGT | |
| AGCACAATGGACTGAGGTTATAACTGTTGATAAAAGGGA | |
| GTGAGTACTTAGAAGTAACATTCATTTTATCATCCATAA | |
| AAATGCTCAACTTCTAAAACACCCACACAGTGAAAAGAC | |
| AGTCTGGTAAATCCAATATTTGTAAAAACTATGCACAAA | |
| ACGCACAGTATTTGGGAAAAGAGGAAAGATTTAT. | |

OST19 (m1y0-66.8)

OST19 is a novel 782 bp gene that is represented by the following nucleotide sequence:

| | |
|---|---|
| AAATGAACACATGTATTTCACAGTACTGCTTTATACAGA | (SEQ ID:19) |
| ATGCTAAGAGCACTTTTTTCTTCATTTTTTTTTTTTTTT | |
| TTTAAAACATATGGACTTTCTTTAATATACCAAAGTACA | |
| TCCAATTCTAATTCCTAAATAAATGTTACAATTCATTGA | |
| ACATTTAAAGGCTTAATTTTTTTTTCCGGGGGAGGAAAG | |
| GATTAAGTAAGTTTATTTTACAAACTGGCTGAAAAATAA | |
| CTTCAAGAAAGTCAATAGAATTCAGTACGGTCTCATCCT | |
| CCCTGACGAAGGTGGTGCCCGATTTGGAGGCGTGATGGC | |
| TATGTCCAAATAATCCCCTATTTGGAACTTCTGTGACTG | |
| CAGGGTCATGGAGTCATCAGTGCCCTTCCTGCCAGACAT | |
| GGTGCTGCCAATCTCTTTAACTCGATATCCAGGTCTTTT | |
| AAGATCCATAAAAACAATTGCAAAATTGAAGTGCGTGCC | |
| CTTCTTTCTGGCTTCTGGGTAGACTTCCTTTACTAAACT | |
| AGTCAGTTCTTTCAAGGTTGCATCCATCCAGGTGTAGAT | |
| CTGCAGCTCGCTCGAAGGCACGTTCCCGCGGGAGAACTC | |
| GTCCATTCGGTGGTGGCGGCCGTTGTTGGTGGTGAAGAC | |
| CCGCAGCAGCAGCGGGCAGGTCTTCTCGCGGTCGATCGG | |
| CTTCTCCGGCTCCTTCTTAATTTCCTCCTGGGTAACGCG | |
| CGATTCCACCGCCATCTTCCTCCTTCGGCCCGGGAGACG | |
| CTCGCCCTGACCTACGACCCCTGCGGCGAGCCTCGTGCC | |
| GA. | |

The open reading frame of OST19 encodes a 153 amino acid polypeptide shown below: MAVESRVTQEEIKKEPEKPIDREKTCPLLLRVFTTNNGRHHRMDEFSRGNVPSSELQIYTWMDATLKELTSLVKEVYP EARKKGTHFNFAIVFMDLKRPGYRVKEIGSTMSGRKGTDDSMTLQSQKFQIGDYLDIAITPPNRAPPSSGRMRPY (SEQ ID: 47).

OST20 (p0t0-69.6)

OST20 is a novel 69 bp gene fragment. The nucleic acid has the following sequence: ACGCGTGAGAGACGTTCCTCGAGCCGAATTTTGTCGGGAGGACCCGGCAACGGACAGTCAAAAAGATCN (SEQ ID: 20).

OST21 (u0f0141.9)

OST21 is a novel 493 bp gene fragment. The nucleic acid has the following sequence:

| | |
|---|---|
| TTTTTTTTTTTTTTTTTTCATTAAAAAATAGTGCTCTTT | (SEQ ID:21) |
| ATTATAAATTACTGAAATGTTTCTTTTCTGAGTATAAAT | |
| ATAAATATTATGTGCAAAGTCTGAGTTGAACTGGGATTT | |
| GGTTGAGTTTTTTCAAGCATCTTCAGACAGCCTCGAAGG | |
| CCTGAGTGGGAAGAGGGGCGGGCTGGAGGTGGATCTTTT | |
| TATAAAAGTGATTGAGGCAAATTGTAAACATTATCCAAA | |
| ACAACAGAGAGAAAAAAGCCACCCCAACACACAGCTGCC | |
| CCACCCAGCCCCATACCTGACTCAGAAAACTGTTGTTTC | |
| TCCCCAACACAGGACCAGTTCCAGGTAATTCCATGGCTG | |
| CTACCTTCCTGTGAGGCCGAGGTGAGCGGCATGTGAAGG | |
| CTTCTCCATCGCTCCCCGCCTCCCCCCAGGTGAGAGGGG | |
| GAGGGCTGTGCAGGGCCTGGAGGCCCTCGGAACTACTGA | |
| CCAAGAGGTGCCTAGGCCTCCAGCC. | |

OST22 (w0c0154.8)

OST22 is a novel 416 bp gene fragment. The nucleic acid has the following sequence:

| | |
|---|---|
| TTTTTTTTTTTTTTTTAAGGAAATGTTACTGTTGAAAGGGAATGAGGCGGGGAGAGCCCCGCCCCCTCACGATGTGGA | (SEQ ID:22) |
| GAGCCCCGCCCCCTCCAGAATACGCAGAAGCAACCTGGCAGTGTCACCTAGCTAGCTTTGAGTCTAAGAGCCAGGCCT | |
| GAATGCTGCAGGATGGGACTGACTTCCAGCCATGATCCAGAAAAACACTTGGGGCAAGGCAGACCATGCGGGCCTTAG | |
| TCCCTCCCAAAGGCTCGCCTCCAGATGCTGGGGCCAGAGTAGGTGCACCAGAAACGTGAGGGTGGGGGTTGGTGGTAT | |
| CTTTGGAAAAGCGCAAAAAGATTTCTTTAGTGGAATCACTTTGCTGAGAGTAACAAACAGGTCTCTGGATTCCCAAGC | |
| TTCCCTCCACCATCCTGCAAAATCCA. | |

OST23 (y0n0-436.5)

OST23 is a novel 439 bp gene fragment. The nucleic acid has the following sequence:

| | |
|---|---|
| TGTACAAAAAGGACAACCAAAAGACCTTTAAACCTAGGCTATACTGGGCAAACCTGGCCACCGTGCTGGGGATAAGG | (SEQ ID:23) |
| TCAATGTTAGGACCAGACAGCAGATTGCCTGAACTTCCAATTCCCTTCTTGGACTTCTGTATGCTTGTCCCCAAAGA | |
| TGATGAATGAACTCGTAAGTGTACCTTCCCTGACCTGAGAACACCCTGCCTGCTCGGGAAGTATTCAGGGGCAGAATT | |
| CTCTGTGAACATGAAGAGATGAATCACACTGTCCTTAAGAAATTCCTCAAAGTCCAGGAACTTGAGCTTTGTATTTTC | |
| AGGAATGCACATCTCTTAAGCACTCGCAAAACAGGAAGGCTCCACACCTCTGGCAGGCCAGGGCCTTTCTCTTCAGCA | |
| TGAGAAAGACAAGGGGACCAGCAGAGTACTTTTCCTCTGGAGGACTAGT. | |

OST24 (f0i0-184.1)

OST24 is a novel 1996 bp gene fragment. The nucleic acid has the following sequence:

| | |
|---|---|
| GGCCGCGAGATTTTTTTTTTTTTTTTTTTTTTCACAGATTATGTTTGTTTTTATTTGATGTTTCCAAATGTAAAAAAG | (SEQ ID:24) |
| TCAAGATCCTTACAATTCTTAAAACAGTTCGAATGCAGCCATCTCATGAGACCATGCAAAGCGATTCGTGCACACATA | |
| ACCATTTCTTCCCTTCACCTTCCCTGTTTCATAGTGTGACTGACAAAAGAACTGGATGGTTCGTTCTTAGTGGGAAGA | |
| TGCCATTTCTTTACGCCGCCACACAAGTCAAGTCTAACATGTTACTTTGCTTTCTTTTCTTTGAACTTCGGAGGCATA | |
| TGGAACTGGAGAATTTAGCTGCCCCATTTTATTCAGCGCACAAATAAGCACACACAGCAGGCTGCAGGCTGCAGGCGG | |
| CTGGGCTGGTAGACATCTCGCTGGTAGACATCTCTCGTGGATTCAATTGATTTGCAAAAAGAACTGAATTGCCTTAGG | |
| TCTTTTCCTTTATAATCTTGCAGTCTGGATTCCAGAATGAGGAACCCTCAGGTGAAGTTTAACAGTGAAGTTGCATTC | |
| TGTCACACTCAGTGACTCGCTATTTGCATAGACTAAAGACAGTTTTGCTACACGGCATTGAAGAAAATCCTGACCCAG | |
| GTGACATGACTTAGTCCAAATGTTTCTGACCAAAGAAATTATACAACAATGTTCATAGGCTAACTTTTAAAGAGATGG | |
| TTGACATTAATACTGTACATATTTTACATGGCAATACACAGTTCAAGAGTTTATAAAACAGGTTCATGCATTATTACC | |
| GCCCAGAGTATCTATCATGTCCACATAACTCCCGGGCACTTGTGATGGTTTTAGCTGGAAGCCAGTATTCTATACAAA | |
| ACAGTCCAGTTTCTGTTGTGTTTAGTACATGGAAAAATTGCAGAGAATCGTGAGAAAACGAAAAGATAGAAAATGTCA | |
| ATCTGTGGGTTTCTTTAAAATGTTTCTTTTAAAACACACCCAGAGTACATGACCTCTGTACAAAGAAAAATAGAAAAG | |
| GTCTGGACGATCACATTTGTTTACGCTACATAATTTAGAATGAACACTACTGGTTGGGTTTTTCTGCTTTGTAACCTA | |
| ATGTTTTTAGTTCTGCTGCATTTGTGGCACGAGATCTCATTTTCCTTCCTTACAGGTAAGGACATTGGCAGCAGCAAC | |
| ATTACAATTTAAAGGTTAACAGGTTACAGATGTCCTAACTGTACTGCGAAAGATCTTTTCCTCTCCCCCCACCCCTTC | |
| ACTTCCTCCATGACTTCCTGAAGGAAATGTAGGTACTTTTCCATGGGGTGGCCCGTTTTGAGAGAGCACAAAGACAAG | |
| GTAACATAGTTCTAGTTCCCTCACACTCATCTGACAAGCTGCTTACTGACACTCAAGACAGTGTCTTAGGCCTAGGAC | |
| AGCCATTTTGTTTAGTGTGATCTAACCTTGGATGCCAGTCAGTGACTGCTGTATCTAAGGTTAGAGTCTAATCCACTG | |
| GTCAACAATGTTGTTGCTAGACTGAAAATATGAAGAGCACTGGCCATGGGAGCAATGTGGCCCCTAACACAGTCTGTG | |
| TCGGAAGGAGCAGGGAGGAAGCAGCCGCAGAGCGTGCTGAACAGACTTCTCTGAACAGTTTCAGAATGAGCTGTGCGG | |
| TGCCACAGGCAAGTTTCTGACAGTTTTCCCTAAAAATGGAGTGATTCATATAAAATCCACCGTGTCAGAGGGGAGGCA | |
| GTGCCTGTGCACTGACATAATTCTTGCTGTGTACAAATTCTCGGATAGAGTAAACTTGAGTGCTTGGTCAAAGGCAAC | |
| TGGCCCGTCCATACAAATCAGCGCAAACCTGCAGCTTACAAGTGGGAGAATGCGACTCTCTTAAACAAGTTTTGGGGC | |
| ACATATATATATTTTTTTAAATACCAAGAATATTTTTCATTAATTCCCAAAGATTTGGTGACTATTTGATACTGTAAG | |
| AAAAAAATATTAGGGCAAAAACATACCCAACAACCAGTCTCGTGCC. | |

OST25 (m0r0131.4)

OST25 is a novel 522 bp gene fragment. The nucleic acid has the following sequence:

| | |
|---|---|
| TTTTTTTTTTTTTTTGTAAGAACCACTTTAAAAATTTTATTCATAAAATTCAAGTTAAATCATACATGCATTTTGGTG | (SEQ ID:25) |
| CAGGATACATCTTACATAGTACAAAAAGATCCACTGATTTCTGTTCTCCAGAGCACTTTGTCTGAGGGAGTAATGCAAC | |
| ACACATGGTCAAAGAAGATTTTACCTGTCGAATATGGAAAACTAAAGAAACACATTTGAATATATCATGACATGTTTT | |
| TCTAGGCAAAATATTTTTCTTTTTATAAATTACCAATTTTAAATTCATCCAATAATAATTATATCTTTGAAATTACTC | |
| TCCTTCTTTATAATATTGCAATCTGGCAAGAAAGGAATTCTAGTATACTTGGCAAGGTATAAGTATTACCCCAGTGGA | |
| ATGATTGAATAAAGTGTTCATAAATCAGCTTCTTGTCTCCTGGAACCTAAAATTTAATCCAAATCTATAAGTTCATAA | |
| TGAAATGGTTTTGCCATGTTGATGCTGAGACACGTTAAGATTGCACACGTATAT. | |

OST26 (y0c0-253)

OST26 is a novel 2123 bp gene fragment. The nucleic acid has the following sequence:

| | |
|---|---|
| TTGATGAAACTTTTCCCATTCTGTGCAGTTTGAGACTTGGAGAGTCGTTATGTCTATGTAGAAGAATGCACTTTCAAC | (SEQ ID:26) |
| AGAGAATGTTGAAAAAGGTCTTGGAGAAAACCTTTACATTTAGATTCTGGCATTCAGTGATACCATGCAAGCTGGGCA | |
| TTGTGGTACTTTGTAGCAAGCAAGACCCTGTGTCCACACTCCCAACAAACAACGAATCCTACTGCAGCACCCAGAGGC | |
| CGTGGTACGTGGTGATGACCGTTCTGAGGCTCACAGTGGCTTAAAGCAGAAACGGGGAAGGAAGGGCTGGTGCTGTAT | |
| TGCTGCCCCTTCATTTTGCTGAGAAAACCTTTAAAGACAATCTTGTAGCAGGGCAGTGGTGGGTGCATGTCTTTAATC | |
| CCAGCACTTGTGAGGCAGAGGCAGGCAGATCTGTGAGTTTAAGGCCAGCCTGGTCTACAGAGTGAGTTCCAGGACAAC | |
| CAGGGCTACATACAGAGAAATTGTCTTGAAAAAAAAAAAAAAAAATAATCTTGGAATTTTTTTTTTTTTTTTTTACAA | |
| AAATAAACAATTTAACTTTATTAAGTCATGACTTCAGCCCTTACATGGATTTGTTTTTTAAAAAAATATCAGTTCAGA | |
| CTATTATTGAAAGTGACTATGCACAATAATAGGAAATGGCCTGCGTGTGCTGCAGACATGGGACACAAAAGGTTGGAT | |
| GCAATCAGCAAAGAGTGCAAAGCACCTGGGAGGAAGTTTCAAATGTCTAGAAAAGTAGCTCAGAGCTCTGGACCACTC | |
| ACCAAATAAAACAAAAAGCAAAAACAAACAAAACAAAAACCCCACTCAGTACATCTGGCAAACAACTTCCCAACAACA | |
| CTGAACTATCTCCTGCGACCCATAAGAACAATTTAAAATACCCAAAGTGCTAAGACCTCATTGGCAGTACTTTAAATC | |
| TGAGTTTTAATGTTAAATATGATTACTCGAATACCCTAAACTGTATGACATGCCTAATAACAATAAGTTACAAATATT | |
| CAACCTAATAACTTAGACATGATATGGTTAATATAACAGACATTGTATCTCAGCTAACCTTTCATGTAAGGTGAGAAT | |
| TAAAAGACTTGTTCATCTGTGCACTCAAATGAGGAATGTGGTATCCTAGCAACAGTGATGACATCCATTATTACAAAG | |
| GCAGGCAGAGCCTGCAAATGTACACGTTACTTAGAGTTACACTTGATTGAAGAAATGATTCTATGTACAAGTGAGTCC | |
| ATATTCAATGTATAAATTTTCAATGGAGCAGGCTTGGAGTCAAGGCAGGTTTAACAAATGCTGTCTGACTTCTCAATA | |
| GCTGGTGAAATGGCTTCTTAAAAGTGAACAAACTAGTCAAGATGGATGGAGGCAGCTCTCGTTTTCCTCTCAGGTGTG | |
| GTGAATTCCATGAAGCATTAATTCCCGAGGAATGTTTGTCTCTGGACCATACAGTTTACAAGCTCTTCTTTCAAAGGC | |
| CGCTACCATTTGTTTCACAACTTCATCAAAGAACAGTGTAGCGAGCTGAGAGTGCAGAAGGGAGCGGAATTCAAAAGA | |
| AATTGAAAAATCCAAGGTACATGTTCTTTGGATAGCCAGGAAAGACTTGGGCTGAATCGCCAACAGTCTCCAGATGAT | |
| TGAAAAGTTTCCCATCAGTACAAGAGGCCTTTACCAAATGTGGTTTTACCAAGGTCACTATTGACGTGTATCGCTCCA | |
| AAACAGGGGGAAACCCAATTTCTAATCGGGTTTTACAATATCCAGATCTCCTTGATAATATATCTGACTTTTTACACC | |
| AAGGAACAAAATGTTTATAATCTTCCATTCCTGATACTACATCATACATTTCCTGCATGGAGTACCCTATAATTCTTC | |
| TCTCTGAATACTCCTTTCGCTTATTTACTAATGGTGTAGTGATTCTGAAGAAGGTTCTTGCACAGATCTCTTTAGGCA | |
| AAACTGAGGCCCACGGTGCAAGGGTTCTGTTCATCAGTATACCACAGGGAGCTAAGTACCTGACGTTGCGCGCAGACC | |
| CTCGCACCCGGGCTTCCGTCACTGTGGTTGACTTCGGACGGCATCCCGATGCCACCCTCGCCAGAGTCCTCTGACTAG | |
| TCCGAGCTGCCCTCGTG. | |

OST27 (g1k0-354.4)

OST27 is a novel 1324 bp gene that is represented by the following nucleotide sequence:

| | |
|---|---|
| TTTTTTTTTTTTTTTTTAGACTGTCTTTTGATGGTCATTTGTCCCAGCCACCCAGAATCCATTATTCATCCACCCTTC | (SEQ ID:27) |
| CATAAGTGTCCCATTCAAGCCACTAGGCCATCTGAGAGCACCTTGCCCTGATTGGAAGCTTAGGAGACAGCTCACAAT | |
| CACGGGGTGGTAATTTTAGCCTGGTATTCACTGGCTGGGGTAGAGTCCATGTTGAGGCTGTGCTAAATATTCATTAGG | |
| GACAGTGTGGCACCTTTTGCAGTTCCTGTCCCCATGAAGGGTGGCCAACTGGAGCCGAAGATCATGGAACAATGGATT | |
| CTGGCCGGCGGGTTAAATAGCCCAAGTGTCATCCTAGAGCATAAAGGAGACCTAGTGTGGTACCTGCCTGAAATAAGG | |
| CCCTAGTGGGTTTTTTTTTTTTTTTTTTTTTTTTTTTTTTGAGGCTCAAAACCTTTATTGGGCCATTGGGCATGTGAC | |
| ATGGTGCTCTCAGAGTTGATCTCCTGAGATAGGAAATAACGGGTAGAGAAAGACAGGGCCCTGGGCACAACTGGTGCA | |
| GGCCTTCAGAGGGACTAGCGGATGCATGAAGGAGAAACGGAAGTGGAAGGGCAGAGGCTGTGGGAAAATGGGCAGTGG | |
| GAAGACTCTGGGGTCCTCCAGTGTTTGAGAGCAAGGTATAGGTAGGCTGGAGATGTGGGGAGGGGAAGCCAAGGGAGC | |
| CTAAGGTGGGGAAGGGGCTTTGGGTTCAGAGTCCTTCTGTCCTGACCTTGGGAGAAGGGCTTTCACTTTCTAGAAAGA | |
| CCCAAGTGTACCGTCTAGCTGGGACTGGCTAGGGTCCTAGTAGCACCACAGATGGGAAATGGTCAAGAGATGGTAGAG | |
| CATGGCGGCGCTAACAGATCCAGCGGATGAACCTGTCGAAGAAGCTGGGCTGGGCTAGACTGTCGAAGTCTTTCTCCC | |
| GGAAGATGGCCGCCCTGTCACCCAGGCTGATCTTGGTAAGTACATCCATGTCCACATCACCACCCACAGCGACCACGG | |
| TGGGCACCACATTCTGCTTACGCATGGAGTGTACGGACTCCTCCAGGCTGTCGTTGCCCGTGACGCCGTCCGTGAGGA | |
| AGACGAAGGACAGCTCCGCGTGACGCCGGGCCCCACCCCGTGCCCCCCGCACCACGTTGTTGATGGCGTGCACGATGC | |
| CTGTGCCCACGTGCGAGAAAGAATTGAGGTAGGTGGTTCTCTCCAGGGCCTCGTGGATGGTGGTCACATTGTAGGTCA | |
| ACGGGAAGGCCACCTGTTGCTGGTTCTGGCTTCCATCTGTTCCGTTCTTGCCTGCTAGACCCGGAGCTCCCTTCCG. | |

The open reading frame of OST27 encodes a 151 amino acid polypeptide shown below:

| | |
|---|---|
| RKGAPGLAGKNGTDGSQNQQQVAFPLTYNVTTIHEALERTTYLNSFSHVGTGIVHAINNVVRGARGGARRHA | (SEQ ID:48) |
| ELSFVFLTDGVTGNDSLEESVHSMRKQNVVPTVVAVGGDVDMDVLTKISLGDRAAIFREKDFDSLAQPSFFDRFIRWI | |
| C. | |

OST28 (w0n0-317.1)

OST28 is a novel 644 bp gene fragment. The nucleic acid has the following sequence:

| | |
|---|---|
| TTTTTTTTTTTTTTTTTTAGGCAAAGGCATGTATATTTTAGAAAAGGTCTTTACATATGTACACAGTTCGTTATAAAT | (SEQ ID:28) |
| ACATAGAAACCAGGGCCTGCCGGCTGCCCACAGATTCCGGGGCTGGGGAGGGGCAGGTGACAATAATTTAGGGGGCAG | |
| GGGATTGCATATATTAACGGGGTCTACCCCGTGGAGCCCCCATAGCCAGCGGGGCCCGAGCACCGATTTCCTGAGTTA | |
| CCAGCTCTCTGGGACAACCAAGCCCCACGGCTTCTAGGCTGGGCATGGCTGTAGCAGGGGAGGCACAGGGGCCCCACG | |
| CCCCACTGGTTGGGAAGGCACCGTGGCCAGTCACCCTCAGTAGTCATGGGAACCCCTQCTAGCAGAGGAGCCCATAGG | |
| CCTTACTGCCCAGTGCAGTGTAATCCAGAGGCTGATCTAGCAGCCTAAGAGTTTGGGAAGGGTGGACTGAAGTGGATA | |
| ACGCTCTGCCGCTCTGTGCCACCGCCTCCGCCGCCGCCCACGCCCACCAGAGGCGCAGGGGTCTCAGTAAGCCCCGCG | |
| CGCAAAGAGCGGACGATGTCCTCCAGCACTTCCCTGIAATTGATGTCGCAGCCCTGGTGTGCCAGCTCCACAGGGTTG | |
| GTAGCGTTGTCCCTGGTACC. | |

OST29 (Y0i0406.9)

OST29 is a novel 2123 bp gene fragment. The nucleic acid has the following sequence:

| | |
|---|---|
| TTGATGAAACTTTTCCCATTCTGTGCAGTTTGAGACTTGGAGAGTCGTTATGTCTATGTAGAAGAATGCACTTTCAAC | (SEQ. ID:29) |
| AGAGAATGTTGAAAAAGGTCTTGGAGAAAACCTTTACATTTAGATTCTGGCATTCAGTGATACCATGCAAGCTGGGCA | |
| TTGTGGTACTTTGTAGCAAGCAAGACCCTGTGTCCACACTCCCAACAAACAACGAATCCTACTGCAGCACCCAGAGGC | |
| CGTGGTACGTGGTGATGACCGTTCTGAGGCTCACAGTGGCTTACAGCAGAACGGGGAAGGAAAGGGCTGGTGCTGTAT | |
| TGCTGCCCCTTCATTTTGCTGAGAAAACCTTTAAAGACAATCTTGTAGCAGGGCAGTGGTGGGTGCATGTCTTTAATC | |
| CCAGCACTTGTGAGGCAGAGGCAGGCAGATCTGTGAGTTTAAGGCCAGCCTGGTCTACAGAGTGAGTTCCAGGACAAC | |
| CAGGGCTACATACAGAGAAATTGTCTTGAAAAAAAAAAAAAAAAATAATCTTGGAATTTTTTTTTTTTTTTTTTACAA | |
| AATAAACAATTTAACTTTATTAAGTCATGACTTCAGCCCTTACATGGATTTGTTTTTTAAAAAAAATATCAGTTCAGA | |
| CTATTATTGAAAGTGACTATGCACAATAAATAGGAATGGCCTGCGTGTGCTGCAGACATGGGACACAAAAGGTTGGAT | |
| GCAATCAGCAAAGAGTGCAAAGCACCTGGGAGGAAGTTTCAAATGTCTAGAAAAGTAGCTCAGAGCTCTGGACCACTC | |
| ACCAAATAAAACAAAAAGCAAAAACAAACAAAACAAAAACCCCACTCAGTACATCTGGCAAACAACTTCCCAACAACA | |
| CTGAACTATCTCCTGCGACCCATAAGAACAATTTAAAATACCCAAAGTGCTAAGACCTCATTGGCAGTACTTTAAATC | |
| TGAGTTTTAATGTTAAATATGATTACTCGAATACCCTAAACTGTATGACATGCCTAATAACAATAAGTTACAAATATT | |
| CAACCTAATAACTTAGACATGATATGGTTAATATAACAGACATTGTATCTCAGCTAACCTTTCATGTAAGGTGAGAAT | |
| TAAAAGACTTGTTCATCTGTGCACTCAAATGAGGAATGTGGTATCCTAGCAACAGTGATGACATCCATTATTACAAAG | |
| GCAGGCAGAGCCTGCAAATGTACACGTTACTTAGAGTTACACTTGATTGAAGAAATGATTCTATGTACAAGTGAGTCC | |
| ATATTCAATGTATAAATTTTCAATGGAGCAGGCTTGGAGTCAAGQCAGGTTTAACAAATGCTGTCTGACTTCTCAATA | |
| GCTGGTGAAATGGCTTCTTAAAAGTGAACAAACTAGTCAAGATGGATGGAGGCAGCTCTCGTTTTCCTCTCAGGTGTG | |
| GTGAATTCCATGAAGCATTAATTCCCGAGGAATGTTTGTCTCTGGACCATACAGTTTACAAGCTCTTCTTTCAAAGGC | |
| CGCTACCATTTGTTTCACAACTTCATCAAAGAACAGTGTAGCGAGCTGAGAGTGCAGAAGGGAGCGGAATTCAAAAGA | |
| AATTGAAAAATCCAAGGTACATGTTCTTTGGATAGCCAGGAAGACTTGGGCTGAATCGCCAAACAGTCTCCAGATGAT | |
| TGAAAAGTTTCCCATCAGTACAAGAGGCCTTTACCAAATGTGGTTTTACCAAGGTCACTATTGACGTGTATCGCTCCA | |
| AAACAGGGGGAAACCCAATTTCTAATCGGGTTTTACAATATCCAGATCTCCTTGATAATATATCTGACTTTTTACACC | |
| AAGGAACAAAATGTTTATAATCTTCCATTCCTGATACTACATCATACATTTCCTGCATGGAGTACCCTATAATTCTTC | |
| TCTCTGAATACTCCTTTCGCTTATTTACTAATGGTGTAGTGATTCTGAAGAAGGTTCTTGCACAGATCTCTTTAGGCA | |
| AAACTGAGGCCCACGGTGCAAGGGTTCTGTTCATCAGTATACCACAGGGAGCTAAGTACCTGACGTTGCGCGCAGACC | |
| CTCGCACCCGGGCTTCCGTCACTGTGGTTGACTTCGGACGQCATCCCGATGCCACCCTCGCCAGAGTCCTCTGACTAG | |
| TCCGAGCTGCCCTCGTG. | |

OST30 (i0r091.5)

OST30 is a novel 389 bp gene fragment. The nucleic acid has the following sequence:

| | |
|---|---|
| AGTCACTCCTATGTTCTCAACAAAACCAGGGCAGCAGCTGAAGTGGGAATCAACAGTGAGACAATTGTGAAACCAGCC | (SEQ ID:30) |
| TCAGTGTCAGAGGAAGAGCTGTTGANTTCAATCAGGACATTGAATGACGATGAGAATGTAGATGGCCTCCTTGTTCAG | |
| CTGCCACTCCCAGAGCACATTGACGAGAGAAAGATCTGCAATGCTGTTTCTCCTGACAAGGATGTTGATGGCTTTCAT | |
| GTCATCAACGTTGGGCGCATGTTTTTGGACCAGTATTCCATGCTTGCCCACAACTCCATGGGGGCGTTGGGAGATACT | |
| CAAGCGAACAGGCATTCCAACCTTAGGGAAGAATGTGGTAGTGGCTGGCAGGTCCAAAAACGTTGGGATGCCAATTG. | |

OST31 (r0t0-263.3)

OST31 is a novel 605 bp gene that is represented by the following nucleotide sequence:

| | |
|---|---|
| GCAGAGATTCCATCATAATCAGCATGAAGAACNTGTAATTAGTAAATCCCACACAGTTATTCACCCATGGCCAGTGAT | (SEQ ID: 31) |
| GATCCATCTTAAGAACACATCTGTCACATGCTGAGCAGTGATGGGCCCGGTCAGGTTTAATCAACTGACATTTTTCAC | |
| AATATCGGATAGCTTTTGAAGCTGACGTGGTGTAGACAGGCAAGTCTCTTGCTGCTCTTCTCAAAATGTCTTGCTGTC | |
| TTTCTTGGCTGAATTCCTTCTCATAACGTTCCTTCTCAGAGTTTGACAAGTAGAACTCTTTGGAGGGGCTAGCGGGAG | |
| ATGTGAAAATTGTCATCCAATAGGACCATACAAACATAACAAAGAACAGATGGAAAGCCACAAGGTAAACAACGGTCT | |
| TTCCTTTTTCTCCTGTTCTGGAAACAGTACTCACGCACAGCTCCACCACGTACGCGTAGTAGGACCAGACGACCACAA | |
| AGGTGATGAAGAGCACCGGAACCCAGCCCACTACGCGTTGGCAGCAGCGCCAGAGTTGTCCAGGGCGCCATGTTCTTC | |
| TGCTGATTGCCTAGTCCAGCAGCCCACGGTTCTGCAGGGCCGCAGGGGTCTTGGGGCCC. | |

The open reading frame of OST32 encodes a 185 amino acid polypeptide shown below:

| | |
|---|---|
| AISRRTWRPGQLWRCCQRVVGWVPVLFITFVVVWSYYAYVVELCVSTVSRTGEKGKTVVYLVAFHLFFVMFVWSYWMT | (SEQ ID: 49) |
| IFTSPASPSKEFYLSNSEKERYEKEFSQERQQDILRRAARDLPVYTTSASKAIRYCEKCQLIKPDRAHHCSACDRCVL | |
| KMDHHWPWVNNCVGFTNYXFFMLIMMESL. | |

OST32 (s0w0252.9)

OST32 is a novel 655 bp gene fragment. The nucleic acid has the following sequence:

| | |
|---|---|
| TTTTTTTTTTTTTTTTTTGTTTCAATAAGTTTATTGTGAAATTCAAAGCTTCCACGATAGCGTCCTTTAGAAAATGAA | (SEQ ID: 32) |
| AGCATTGCCCGGGATCCGTTTTTGAAAGAGCAGTGCAGTCTGCTAAGTCCGGCTCGCTCCTGCAGTGGCCTCTCCTGC | |
| AGCTGTACCAAGTCCAGGCGGCCTCCTGCCTTGCCTTTCCCCTCCTTTCACTGAAGGCTGCTTGCTGAGTCACACCTG | |
| CCTCGCTGGTCGGTTCTTCCCGGCCCTGGGGCCAACGTCCTTGCCTAGGCCTTCGTGTGCTAGCAGCCTCTCCGGACA | |
| CTGGCAGAGTCCTGGGCTCGGAACAGGATGTCCCCGCTGCCGCCAGACCCCCTTCTTGGGAGGCCGCGCTCCTCGCTC | |
| TCCAGGCAACATCGCCTGCCAGTCTCCATGCGCACTCGAGCGCCCTCGGAGGGCCACTCCAGAGCCAGCAGCGCGCTA | |
| GAGTGTCACCAAGTCGATCAGACCAAGGTCCCTGCCAGGCTGTCACTCGGGAAGGGTGATGCTGGGCACCCAGTCGTT | |
| GAAGCTGCGGCTCTCCTCGCAGAACAAACTGAGCTTCTCCAAGGCAGGGTCCTTCCATGTGTCTTCATTAGGGTTCGA | |
| AATGAGGATGCAATGCAAGTCTCCCGGCGCC. | |

General Screeening and Diagnostic Methods Using OST Sequences

Several of the herein disclosed methods relate to comparing the levels of expression of one or more OST nucleic acids in a test and reference cell populations. The sequence information disclosed herein, coupled with nucleic acid detection methods known in the art, allow for detection and comparison of the various OST transcripts. In some embodiments, the OST nucleic acids and polypeptide correspond to nucleic acids or polypeptides which include the various sequences (referenced by SEQ ID NOs) disclosed for each OST nucleic acid sequence.

In its various aspects and embodiments, the invention includes providing a test cell population which includes at least one cell that is capable of expressing one or more of the sequences OST 1-48, or any combination of OST sequences thereof. By “capable of expressing” is meant that the gene is present in an intact form in the cell and can be expressed. Expression of one, some, or all of the OST sequences is then detected, if present, and, preferably, measured. Using sequence information provided by the database entries for the known sequences, or the sequence information for the newly described sequences, expression of the OST sequences can be detected (if expressed) and measured using techniques well known to one of ordinary skill in the art. For example, sequences within the sequence database entries corresponding to OST sequences, or within the sequences disclosed herein, can be used to construct probes for detecting OST RNA sequences in, e.g., northern blot hybridization analyses or methods which specifically, and, preferably, quantitatively amplify specific nucleic acid sequences. As another example, the sequences can be used to construct primers for specifically amplifying the OST sequences in, e.g., amplification-based detection methods such as reverse-transcription based polymerase chain reaction. When alterations in gene expression are associated with gene amplification or deletion, sequence comparisons in test and reference populations can be made by comparing relative amounts of the examined DNA sequences in the test and reference cell populations.

For OST sequences whose polypeptide product is known, expression can be also measured at the protein level, i.e., by measuring the levels of polypeptides encoded by the gene products described herein. Such methods are well known in the art and include, e.g., immunoassays based on antibodies to proteins encoded by the genes.

Expression level of one or more of the OST sequences in the test cell population is then compared to expression levels of the sequences in one or more cells from a reference cell population. Expression of sequences in test and control populations of cells can be compared using any art-recognized method for comparing expression of nucleic acid sequences. For example, expression can be compared using GENECALLING® methods as described in U.S. Pat. No. 5,871,697 and in Shimkets et al., Nat. Biotechnol. 17:798-803.

In various embodiments, the expression of 2, 3, 4, 5, 6, 7, 8, 9, 10, 15, 20, 25, 28, 30, 35, 40, or all of the sequences represented by OST 1-48 are measured. If desired, expression of these sequences can be measured along with other sequences whose expression is known to be altered according to one of the herein described parameters or conditions.

The reference cell population includes cells one or more cells capable of expressing the measured OST sequences and for which the compared parameter is known, e.g., parathyroid hormone status, osteoregenerative agent exposure status. By “parathyroid hormone status” is meant that is known whether the reference cell has been exposed to a parathyroid hormone. By i “osteoregenerative agent exposure status” is meant that it is know whether the reference cell has been exposed to a osteoregenerative agent. Whether or not comparison of the gene expression profile in the test cell population to the reference cell population reveals the presence, or degree, of the measured parameter depends on the composition of the reference cell population. For example, if the reference cell population is composed of cells that have not been treated with parathyroid hormonr, a similar gene expression level in the test cell population and a reference cell population indicates the test agent is not a parathyroid hormone receptor ligand. Conversely, if the reference cell population is made up of cells that have been treated with a parathyroid hormone, a similar gene expression profile between the test cell population and the reference cell population indicates the test agent is a parathyroid hormone receptor ligand.

In various embodiments, a OST sequence in a test cell population is considered comparable in expression level to the expression level of the OST sequence in the reference cell population if its expression level varies within a factor of 2.0, 1.5, or 1.0 fold to the level of the OST transcript in the reference cell population. In various embodiments, a OST sequence in a test cell population can be considered altered in levels of expression if its expression level varies from the reference cell population by more than 1.0, 1.5, 2.0 or more fold from the expression level of the corresponding OST sequence in the reference cell population. In some embodiments, the variation in expression of a particular OST sequence corresponds to the change in expression level observed for the OST sequence in the presence and absence of the parathyroid hormone receptor as shown in Tables 1 and 2.

If desired, comparison of differentially expressed sequences between a test cell population and a reference cell population can be done with respect to a control nucleic acid whose expression is independent of the parameter or condition being measured. Expression levels of the control nucleic acid in the test and reference nucleic acid can be used to normalize signal levels in the compared populations. Suitable control nucleic acids can readily be determined by one of ordinary skill in the art.

In some embodiments, the test cell population is compared to multiple reference cell populations. Each of the multiple reference populations may differ in the known parameter. Thus, a test cell population may be compared to a first reference cell population known to have been exposed to a parathyroid hormone or a osteoregenerative agent, as well as a second reference population known have not been exposed to a parathyroid hormone or a osteoregenerative agent.

The test cell population that is exposed to, i.e., contacted with, the test parathyroid hormone or a osteoregenerative agent can be any number of cells, i.e., one or more cells, and can be provided in vitro, in vivo, or ex vivo.

In other embodiments, the test cell population can be divided into two or more subpopulations. The subpopulations can be created by dividing the first population of cells to create as identical a subpopulation as possible. This will be suitable, in, for example, in vitro or ex vivo screening methods. In some embodiments, various sub populations can be exposed to a control agent, and/or a test agent, multiple test agents, or, e.g., varying dosages of one or multiple test agents administered together, or in various combinations.

Preferably, cells in the reference cell population are derived from a tissue type as similar as possible to test cell, e.g., bone cells such as osteoclast or osteoblasts. In some embodiments, the control cell is derived from the same subject as the test cell, e.g., from a region proximal to the region of origin of the test cell. In other embodiments, the reference cell population is derived from a plurality of cells. For example, the reference cell population can be a database of expression patterns from previously tested cells for which one of the herein-described parameters or conditions is known.

The subject is preferably a mammal. The mammal can be, e.g., a human, non-human primate, mouse, rat, dog, cat, horse, or cow.

Screening for Parathyroid Hormone Receptor Ligands

In one aspect, the invention provides a method of identifying parathyroid hormone receptor ligands. The parathyroid hormone receptor ligand can be identified by providing a cell population that includes cells capable of expressing one or more nucleic acid sequences homologous to those listed in Tables 1 and 2 as OST 1-48. The sequences need not be identical to sequences including OST 1-48 so as long as the sequence is sufficiently similar that specific hybridization can be detected. Preferably, the cell includes sequences that are identical, or nearly identical to those identifying the OST nucleic acids shown in Tables 1 and 2.

Expression of the nucleic acid sequences in the test cell population is then compared to the expression of the nucleic acid sequences in a reference cell population, which is a cell population that has not been exposed to the test agent, or, in some embodiments, a cell population exposed the test agent. Comparison can be performed on test and reference samples measured concurrently or at temporally distinct times. An example of the latter is the use of compiled expression information, e.g., a sequence database, which assembles information about expression levels of known sequences following administration of various agents. For example, alteration of expression levels following administration of test agent can be compared to the expression changes observed in the nucleic acid sequences following administration of a control agent, parathyroid hormone.

An alteration in expression of the nucleic acid sequence in the test cell population compared to the expression of the nucleic acid sequence in the reference cell population that has not been exposed to the test agent indicates the test agent is a parathyroid hormone receptor ligand.

The invention also includes a parathyroid hormone receptor ligand identified according to this screening method, and a pharmaceutical composition which includes the parathyroid hormone receptor ligands.

Screening for Osteoregenterative Agents

In one aspect, the invention provides a method screening for osteoregenerative agents. By “osteoregenerative agent” is meant an agent that promotes bone regeneration or remodeling, i.e., bone formation or bone resorbtion. Preferably, the agent promotes bone formation. For example an osteoregenerative agent is parathyroid hormone. The osteoregenerative agent can be identified by providing a cell population that includes cells capable of expressing one or more nucleic acid sequences homologous to those listed in Tables 1 and 2 as OST 1-48. The sequences need not be identical to sequences including OST 1-48 so as long as the sequence is sufficiently similar that specific hybridization can be detected. Preferably, the cell includes sequences that are identical, or nearly identical to those identifying the OST nucleic acids shown in Tables 1 and 2.

Expression of the nucleic acid sequences in the test cell population is then compared to the expression of the nucleic acid sequences in a reference cell population, which is a cell population that has not been exposed to the test agent, or, in some embodiments, a cell population exposed the test agent. Comparison can be performed on test and reference samples measured concurrently or at temporally distinct times. An example of the latter is the use of compiled expression information, e.g., a sequence database, which assembles information about expression levels of known sequences following administration of various agents. For example, alteration of expression levels following administration of test agent can be compared to the expression changes observed in the nucleic acid sequences following administration of a control agent, parathyroid hormone.

An alteration in expression of the nucleic acid sequence in the test cell population compared to the expression of the nucleic acid sequence in the reference cell population that has not been exposed to the test agent indicates the test agent is an osteoregenerative agent.

The invention also includes the osteoregenerative agent identified according to this screening method, and a pharmaceutical composition which includes the osteoregenerative agent.

Screening Assays for Identifying a Candidate Therapeutic Agent for Treating or Preventing Bone Disorder

The differentially expressed sequences disclosed herein can also be used to identify candidate therapeutic agents for treating bone disorder. The method is based on screening a candidate therapeutic agent to determine if it induces an expression profile of one or more OST 1-48 sequences, sequences in a test cell population.

In the method, a test cell population is exposed to a test agent or a combination of test agents (sequentially or consequentially), and the expression of one or more of the OST sequences is measured. The expression of the OST sequences in the test population is compared to expression level of the OST sequences in a reference cell population whose osteoregenerative agent status is known. If the reference cell population contains cells that have not been exposed to a osteoregenerative agent, alteration of expression of the nucleic acids in the test cell population as compared to the reference cell population indicates that the test agent is a candidate therapeutic agent.

In some embodiments, the reference cell population includes cells that have been exposed to a test agent. When this cell population is used, an alteration in expression of the nucleic acid sequences in the presence of the agent from the expression profile of the cell population in the absence of the agent indicates the agent is a candidate therapeutic agent. In other embodiments, the test cell population includes cells that have not been exposed to a osteoregenerative agent. For this cell population, a similarity in expression of the OST sequences in the test and control cell populations indicates the test agent is not a candidate therapeutic agent, while a difference suggests it is a candidate.

The test agent can be a compound not previously described or can be a previously known compound but which is not known to be a parathyroid hormone ligand.

An agent effective in stimulating expression of underexpressed genes, or in suppressing expression of overexpressed genes can be further tested for its ability to prevent the bone disorder e.g. osteoporosis, and as a potential therapeutic useful for the treatment of such pathophysiology. Further evaluation of the clinical usefulness of such a compound can be performed using standard methods of evaluating toxicity and clinical effectiveness of anti-diabetic agents.

Assessing Osteoregenerative Activity of an Agent in a Subject

The differentially expressed OST sequences identified herein also allow for the osteoregenerative activity of an osteoregenerative agent to be determined or monitored. In this method, a test cell population from a subject is exposed to a test agent, i.e. a osteoregenerative agent. If desired, test cell populations can be taken from the subject at various time points before, during, or after exposure to the test agent. Expression of one or more of the OST sequences, e.g., OST 1-48, in the cell population is then measured and compared to a reference cell population which includes cells whose exposure status to an osteoregenerative agent is known. Preferably, the reference cells not been exposed to the test agent.

If the reference cell population contains no cells exposed to the treatment, a similarity in expression between OST sequences in the test cell population and the reference cell population indicates that the treatment is non-osteoregenerative. However, a difference in expression between OST sequences in the test population and this reference cell population indicates the treatment is osteoregenerative.

Selecting a Therapeutic Agent for Treating a Bone Disorder that is Appropriate for a Particular Individual

Differences in the genetic makeup of individuals can result in differences in their relative abilities to metabolize various drugs. An agent that is metabolized in a subject to act as a osteoregenerative agent can manifest itself by inducing a change in gene expression pattern from that characteristic of a pathophysiologic state to a gene expression pattern characteristic of a non-pathophysiologic state. Accordingly, the differentially expressed OST sequences disclosed herein allow for a putative therapeutic or prophylactic agent to be tested in a test cell population from a selected subject in order to determine if the agent is a suitable osteoregenerative agent in the subject.

To identify a osteoregenerative agent, that is appropriate for a specific subject, a test cell population from the subject is exposed to a therapeutic agent, and the expression of one or more of OST 1-48 sequences is measured.

In some embodiments, the test cell population contains an bone cell, e.g., osteoblast or osteocyte. In other embodiments, the agent is first mixed with a cell extract, e.g., an bone cell extract, which contains enzymes that metabolize drugs into an active form. The activated form of the therapeutic agent can then be mixed with the test cell population and gene expression measured. Preferably, the cell population is contacted ex vivo with the agent or activated form of the agent.

Expression of the nucleic acid sequences in the test cell population is then compared to the expression of the nucleic acid sequences a reference cell population. The reference cell population includes at least one cell whose osteoregenerative agent status is known. If the reference cell had been exposed to a osteoregenerative agent a similar gene expression profile between the test cell population and the reference cell population indicates the agent is suitable for treating the pathophysiology in the subject. A difference in expression between sequences in the test cell population and those in the reference cell population indicates that the agent is not suitable for treating the bone disorder in the subject.

If the reference cell has not been exposed to a osteoregenerative agent, a similarity in gene expression patterns between the test cell population and the reference cell population indicates the agent is not suitable for treating the bone disorder in the subject, while a dissimilar gene expression patterns indicate the agent will be suitable for treating the subject.

In some embodiments, a decrease in expression of one or more of the sequences OST: 1-48 or an increase in expression of one or more of the sequences OST: 1-48 in a test cell population relative to a reference cell population is indicative that the agent is therapeutic.

The test agent can be any compound or composition. In some embodiments the test agents are compounds and composition know to be parathyroid hormone ligands or osteoregenerative agents.

Methods of Diagnosing or Determining the Susceptibility of a Bone Disorder in a Subject

The invention further provides a method of diagnosing a bone disorder, e.g., bone formation disorder or a bone resorbtion disorder, in a subject. Example of bone disorders include for example, osteopenia, osteopetrosis or osteoporosis. A disorder is diagnosed by examining the expression of one or more OST nucleic acid sequences from a test population of cells from a subject suspected of have the disorder.

Expression of one or more of the OST nucleic acid sequences, e.g. OST: 1-48 is measured in the test cell and compared to the expression of the sequences in the reference cell population. The reference cell population contains at least one cell whose, or disease status (i.e., the reference cell population is from a subject suffering from a bone disorder, e.g., osteoporosis) is known. If the reference cell population contains cells that have not suffering from a bone disorder, then a similarity in expression between OST sequences in the test population and the reference cell population indicates the subject does not have a bone disorder. A difference in expression between OST sequences in the test population and the reference cell population indicates the reference cell population has a bone disorder.

Conversely, when the reference cell population contains cells that have a bone disorder, a similarity in expression pattern between the test cell population and the reference cell population indicates the test cell population has a bone disorder. A difference in expression between OST sequences in the test population and the reference cell population indicates the subject does not have a bone disorder.

Methods of Treating Bone Disorders in a Subject

Also included in the invention is a method of treating, i.e, preventing or delaying the onset of a bone disorder in a subject by administering to the subject an agent which modulates the expression or activity of one or more nucleic acids selected from the group consisting of OST 1-48. “Modulates” is meant to include increase or decrease expression or activity of the OST nucleic acids. Preferably, modulation results in alteration alter the expression or activity of the OST genes or gene products in a subject to a level similar or identical to a subject not suffering from the bone disorder.

The bone disorder can be any of the pathophysiologies described herein, e.g., steopenia, osteopetrosis or osteoporosis. The subject can be, e.g., a human, a rodent such as a mouse or rat, or a dog or cat.

The herein described OST nucleic acids, polypeptides, antibodies, agonists, and antagonists when used therapeutically are referred to herein as “Therapeutics”. Methods of administration of Therapeutics include, but are not limited to, intradermal, intramuscular, intraperitoneal, intravenous, subcutaneous, intranasal, epidural, and oral routes. The Therapeutics of the present invention may be administered by any convenient route, for example by infusion or bolus injection, by absorption through epithelial or mucocutaneous linings (e.g., oral mucosa, rectal and intestinal mucosa, etc.) and may be administered together with other biologically-active agents. Administration can be systemic or local. In addition, it may be advantageous to administer the Therapeutic into the central nervous system by any suitable route, including intraventricular and intrathecal injection. Intraventricular injection may be facilitated by an intraventricular catheter attached to a reservoir (e.g., an Ommaya reservoir). Pulmonary administration may also be employed by use of an inhaler or nebulizer, and formulation with an aerosolizing agent. It may also be desirable to administer the Therapeutic locally to the area in need of treatment; this may be achieved by, for example, and not by way of limitation, local infusion during surgery, topical application, by injection, by means of a catheter, by means of a suppository, or by means of an implant. In a specific embodiment, administration may be by direct injection at the site (or former site) of a malignant tumor or neoplastic or pre-neoplastic tissue.

Various delivery systems are known and can be used to administer a Therapeutic of the present invention including, e.g.: (i) encapsulation in liposomes, microparticles, microcapsules; (ii) recombinant cells capable of expressing the Therapeutic; (iii) receptor-mediated endocytosis (See, e.g., Wu and Wu, 1987. J Biol Chem 262:4429-4432); (iv) construction of a Therapeutic nucleic acid as part of a retroviral or other vector, and the like. In one embodiment of the present invention, the Therapeutic may be delivered in a vesicle, in particular a liposome. In a liposome, the protein of the present invention is combined, in addition to other pharmaceutically acceptable carriers, with amphipathic agents such as lipids which exist in aggregated form as micelles, insoluble monolayers, liquid crystals, or lamellar layers in aqueous solution. Suitable lipids for liposomal formulation include, without limitation, monoglycerides, diglycerides, sulfatides, lysolecithin, phospholipids, saponin, bile acids, and the like. Preparation of such liposomal formulations is within the level of skill in the art, as disclosed, for example, in U.S. Pat. No. 4,837,028; and U.S. Pat. No. 4,737,323, all of which are incorporated herein by reference. In yet another embodiment, the Therapeutic can be delivered in a controlled release system including, e.g.: a delivery pump (See, e.g., Saudek, et al, 1989. New Engl J Med 321:574 and a semi-permeable polymeric material (See, e.g., Howard, et al., 1989. J Neurosurg 71:105). Additionally, the controlled release system can be placed in proximity of the therapeutic target (e.g., the brain), thus requiring only a fraction of the systemic dose. See, e.g., Goodson, In: Medical Applications of Controlled Release 1984. (CRC Press, Bocca Raton, Fla.).

In a specific embodiment of the present invention, where the Therapeutic is a nucleic acid encoding a protein, the Therapeutic nucleic acid may be administered in vivo to promote expression of its encoded protein, by constructing it as part of an appropriate nucleic acid expression vector and administering it so that it becomes intracellular (e.g., by use of a retroviral vector, by direct injection, by use of microparticle bombardment, by coating with lipids or cell-surface receptors or transfecting agents, or by administering it in linkage to a homeobox-like peptide which is known to enter the nucleus (See, e.g., Joliot, et al., 1991. Proc Natl Acad Sci USA 88:1864-1868), and the like. Alternatively, a nucleic acid Therapeutic can be introduced intracellularly and incorporated within host cell DNA for expression, by homologous recombination.

As used herein, the term “therapeutically effective amount” means the total amount of each active component of the pharmaceutical composition or method that is sufficient to show a meaningful patient benefit, i.e., treatment, healing, prevention or amelioration of the relevant medical condition, or an increase in rate of treatment, healing, prevention or amelioration of such conditions. When applied to an individual active ingredient, administered alone, the term refers to that ingredient alone. When applied to a combination, the term refers to combined amounts of the active ingredients that result in the therapeutic effect, whether administered in combination, serially or simultaneously.

The amount of the Therapeutic of the invention which will be effective in the treatment of a particular disorder or condition will depend on the nature of the disorder or condition, and may be determined by standard clinical techniques by those of average skill within the art. In addition, in vitro assays may optionally be employed to help identify optimal dosage ranges. The precise dose to be employed in the formulation will also depend on the route of administration, and the overall seriousness of the disease or disorder, and should be decided according to the judgment of the practitioner and each patient's circumstances. Ultimately, the attending physician will decide the amount of protein of the present invention with which to treat each individual patient. Initially, the attending physician will administer low doses of protein of the present invention and observe the patient's response. Larger doses of protein of the present invention may be administered until the optimal therapeutic effect is obtained for the patient, and at that point the dosage is not increased further. However, suitable dosage ranges for intravenous administration of the Therapeutics of the present invention are generally about 20-500 micrograms (μg) of active compound per kilogram (Kg) body weight. Suitable dosage ranges for intranasal administration are generally about 0.01 pg/kg body weight to 1 mg/kg body weight. Effective doses may be extrapolated from dose-response curves derived from in vitro or animal model test systems. Suppositories generally contain active ingredient in the range of 0.5% to 10% by weight; oral formulations preferably contain 10% to 95% active ingredient.

The duration of intravenous therapy using the pharmaceutical composition of the present invention will vary, depending on the severity of the disease being treated and the condition and potential idiosyncratic response of each individual patient. It is contemplated that the duration of each application of the protein of the present invention will be in the range of 12 to 24 hours of continuous intravenous administration. Ultimately the attending physician will decide on the appropriate duration of intravenous therapy using the pharmaceutical composition of the present invention.

Polynucleotides of the present invention can also be used for gene therapy. Gene therapy refers to therapy that is performed by the administration of a specific nucleic acid to a subject. Delivery of the Therapeutic nucleic acid into a mammalian subject may be either direct (i.e., the patient is directly exposed to the nucleic acid or nucleic acid-containing vector) or indirect (i.e., cells are first transformed with the nucleic acid in vitro, then transplanted into the patient). These two approaches are known, respectively, as in vivo or ex vivo gene therapy. Polynucleotides of the invention may also be administered by other known methods for introduction of nucleic acid into a cell or organism (including, without limitation, in the form of viral vectors or naked DNA). Any of the methodologies relating to gene therapy available within the art may be used in the practice of the present invention. See e.g., Goldspiel, et al, 1993. Clin Pharm 12:488-505.

Cells may also be cultured ex vivo in the presence of therapeutic agents or proteins of the present invention in order to proliferate or to produce a desired effect on or activity in such cells. Treated cells can then be introduced in vivo for therapeutic purposes.

Assessing Efficacy of Treatment of a Bone Disorder in a Subject

The differentially expressed OST sequences identified herein also allow for the course of treatment of a bone disorder to be monitored. In this method, a test cell population is provided from a subject undergoing treatment for bone disorder. If desired, test cell populations can be taken from the subject at various time points before, during, or after treatment. Expression of one or more of the OST sequences, e.g., OSTs: 1-48 in the cell population is then measured and compared to a reference cell population which includes cells whose pathophysiologic state is known. Preferably, the reference cells not been exposed to the treatment.

If the reference cell population contains no cells exposed to the treatment, a similarity in expression between OST sequences in the test cell population and the reference cell population indicates that the treatment is efficacious. However, a difference in expression between OST sequences in the test population and this reference cell population indicates the treatment is not efficacious.

By “efficacious” is meant that the treatment leads to a decrease in the pathophysiology in a subject. When treatment is applied prophylactically, “efficacious” means that the treatment retards or prevents a pathophysiology. For example, if the bone disorder is osteoporosis, a “efficacious” treatment is one that increases bone density.

Efficaciousness can be determined in association with any known method for treating the particular pathophysiology.

OST Nucleic Acids

Also provided in the invention are novel nucleic acids that include a nucleic acid sequence selected from the group consisting of OST 1-32, or its complement, as well as vectors and cells including these nucleic acids. Also provided are polypeptides encoded by OST nucleic acid or biologically active portions thereof.

Also included in the invention are nucleic acid fragments sufficient for use as hybridization probes to identify OST-encoding nucleic acids (e.g., OST mRNA) and fragments for use as polymerase chain reaction (PCR) primers for the amplification or mutation of OST nucleic acid molecules. As used herein, the term “nucleic acid molecule” is intended to include DNA molecules (e.g., cDNA or genomic DNA), RNA molecules (e.g., mRNA), analogs of the DNA or RNA generated using nucleotide analogs, and derivatives, fragments and homologs thereof. The nucleic acid molecule can be single-stranded or double-stranded, but preferably is double-stranded DNA.

“Probes” refer to nucleic acid sequences of variable length, preferably between at least about 10 nucleotides (nt) or as many as about, e.g., 6,000 nt, depending on use. Probes are used in the detection of identical, similar, or complementary nucleic acid sequences. Longer length probes are usually obtained from a natural or recombinant source, are highly specific and much slower to hybridize than oligomers. Probes may be single- or double-stranded and designed to have specificity in PCR, membrane-based hybridization technologies, or ELISA-like technologies.

An “isolated” nucleic acid molecule is one that is separated from other nucleic acid molecules which are present in the natural source of the nucleic acid. Examples of isolated nucleic acid molecules include, but are not limited to, recombinant DNA molecules contained in a vector, recombinant DNA molecules maintained in a heterologous host cell, partially or substantially purified nucleic acid molecules, and synthetic DNA or RNA molecules. Preferably, an “isolated” nucleic acid is free of sequences which naturally flank the nucleic acid (i.e., sequences located at the 5′ and 3′ ends of the nucleic acid) in the genomic DNA of the organism from which the nucleic acid is derived. For example, in various embodiments, the isolated OST nucleic acid molecule can contain less than about 50 kb, 25 kb, 5 kb, 4 kb, 3 kb, 2 kb, 1 kb, 0.5 kb or 0.1 kb of nucleotide sequences which naturally flank the nucleic acid molecule in genomic DNA of the cell from which the nucleic acid is derived. Moreover, an “isolated” nucleic acid molecule, such as a cDNA molecule, can be substantially free of other cellular material or culture medium when produced by recombinant techniques, or of chemical precursors or other chemicals when chemically synthesized.

A nucleic acid molecule of the present invention, e.g., a nucleic acid molecule having the nucleotide sequence of any of OST 1-32, or a complement of any of these nucleotide sequences, can be isolated using standard molecular biology techniques and the sequence information provided herein. Using all or a portion of these nucleic acid sequences as a hybridization probe, OST nucleic acid sequences can be isolated using standard hybridization and cloning techniques (e.g., as described in Sambrook et al., eds., MOLECULAR CLONING: A LABORATORY MANUAL 2^{nd} Ed., Cold Spring Harbor Laboratory Press, Cold Spring Harbor, N.Y., 1989; and Ausubel, et al., eds., CURRENT PROTOCOLS IN MOLECULAR BIOLOGY, John Wiley & Sons, New York, N.Y., 1993.)

A nucleic acid of the invention can be amplified using cDNA, mRNA or alternatively, genomic DNA, as a template and appropriate oligonucleotide primers according to standard PCR amplification techniques. The nucleic acid so amplified can be cloned into an appropriate vector and characterized by DNA sequence analysis. Furthermore, oligonucleotides corresponding to OST nucleotide sequences can be prepared by standard synthetic techniques, e.g., using an automated DNA synthesizer.

As used herein, the term “oligonucleotide” refers to a series of linked nucleotide residues, which oligonucleotide has a sufficient number of nucleotide bases to be used in a PCR reaction. A short oligonucleotide sequence may be based on, or designed from, a genomic or cDNA sequence and is used to amplify, confirm, or reveal the presence of an identical, similar or complementary DNA or RNA in a particular cell or tissue. Oligonucleotides comprise portions of a nucleic acid sequence having at least about 10 nt and as many as 50 nt, preferably about nt to 30 nt. They may be chemically synthesized and may be used as probes.

In another embodiment, an isolated nucleic acid molecule of the invention comprises a nucleic acid molecule that is a complement of the nucleotide sequence shown in OSTs: 1-32. In another embodiment, an isolated nucleic acid molecule of the invention comprises a nucleic acid molecule that is a complement of the nucleotide sequence shown in any of these sequences, or a portion of any of these nucleotide sequences. A nucleic acid molecule that is complementary to the nucleotide sequence shown in OST 1-32 is one that is sufficiently complementary to the nucleotide sequence shown, such that it can hydrogen bond with little or no mismatches to the nucleotide sequences shown, thereby forming a stable duplex.

As used herein, the term “complementary” refers to Watson-Crick or Hoogsteen base pairing between nucleotides units of a nucleic acid molecule, and the term “binding” means the physical or chemical interaction between two polypeptides or compounds or associated polypeptides or compounds or combinations thereof. Binding includes ionic, non-ionic, Von der Waals, hydrophobic interactions, etc. A physical interaction can be either direct or indirect. Indirect interactions may be through or due to the effects of another polypeptide or compound. Direct binding refers to interactions that do not take place through, or due to, the effect of another polypeptide or compound, but instead are without other substantial chemical intermediates.

Moreover, the nucleic acid molecule of the invention can comprise only a portion of the nucleic acid sequence of OST 1-32 e.g., a fragment that can be used as a probe or primer or a fragment encoding a biologically active portion of OST. Fragments provided herein are defined as sequences of at least 6 (contiguous) nucleic acids or at least 4 (contiguous) amino acids, a length sufficient to allow for specific hybridization in the case of nucleic acids or for specific recognition of an epitope in the case of amino acids, respectively, and are at most some portion less than a full length sequence. Fragments may be derived from any contiguous portion of a nucleic acid or amino acid sequence of choice. Derivatives are nucleic acid sequences or amino acid sequences formed from the native compounds either directly or by modification or partial substitution. Analogs are nucleic acid sequences or amino acid sequences that have a structure similar to, but not identical to, the native compound but differs from it in respect to certain components or side chains. Analogs may be synthetic or from a different evolutionary origin and may have a similar or opposite metabolic activity compared to wild type.

Derivatives and analogs may be full length or other than full length, if the derivative or analog contains a modified nucleic acid or amino acid, as described below. Derivatives or analogs of the nucleic acids or proteins of the invention include, but are not limited to, molecules comprising regions that are substantially homologous to the nucleic acids or proteins of the invention, in various embodiments, by at least about 45%, 50%, 70%, 80%, 95%, 98%, or even 99% identity (with a preferred identity of 80-99%) over a nucleic acid or amino acid sequence of identical size or when compared to an aligned sequence in which the alignment is done by a computer homology program known in the art, or whose encoding nucleic acid is capable of hybridizing to the complement of a sequence encoding the aforementioned proteins under stringent, moderately stringent, or low stringent conditions. See e.g. Ausubel, et al., CURRENT PROTOCOLS IN MOLECULAR BIOLOGY, John Wiley & Sons, New York, N.Y., 1993, and below. An exemplary program is the Gap program (Wisconsin Sequence Analysis Package, Version 8 for UNIX, Genetics Computer Group, University Research Park, Madison, Wis.) using the default settings, which uses the algorithm of Smith and Waterman (Adv. Appl. Math., 1981, 2: 482-489, which in incorporated herein by reference in its entirety).

A “homologous nucleic acid sequence” or “homologous amino acid sequence,” or variations thereof, refer to sequences characterized by a homology at the nucleotide level or amino acid level as discussed above. Homologous nucleotide sequences encode those sequences coding for isoforms of a OST polypeptide. Isoforms can be expressed in different tissues of the same organism as a result of, for example, alternative splicing of RNA. Alternatively, isoforms can be encoded by different genes. In the present invention, homologous nucleotide sequences include nucleotide sequences encoding for a OST polypeptide of species other than humans, including, but not limited to, mammals, and thus can include, e.g., mouse, rat, rabbit, dog, cat cow, horse, and other organisms. Homologous nucleotide sequences also include, but are not limited to, naturally occurring allelic variations and mutations of the nucleotide sequences set forth herein. A homologous nucleotide sequence does not, however, include the nucleotide sequence encoding a human OST protein. Homologous nucleic acid sequences include those nucleic acid sequences that encode conservative amino acid substitutions (see below) in a OST polypeptide, as well as a polypeptide having a OST activity. A homologous amino acid sequence does not encode the amino acid sequence of a human OST polypeptide.

The nucleotide sequence determined from the cloning of human OST genes allows for the generation of probes and primers designed for use in identifying and/or cloning OST homologues in other cell types, e.g., from other tissues, as well as OST homologues from other mammals. The probe/primer typically comprises a substantially purified oligonucleotide. The oligonucleotide typically comprises a region of nucleotide sequence that hybridizes under stringent conditions to at least about 12, 25, 50, 100, 150, 200, 250, 300, 350 or 400 consecutive sense strand nucleotide sequence of a nucleic acid comprising a OST sequence, or an anti-sense strand nucleotide sequence of a nucleic acid comprising a OST sequence, or of a naturally occurring mutant of these sequences.

Probes based on human OST nucleotide sequences can be used to detect transcripts or genomic sequences encoding the same or homologous proteins. In various embodiments, the probe further comprises a label group attached thereto, e.g., the label group can be a radioisotope, a fluorescent compound, an enzyme, or an enzyme co-factor. Such probes can be used as a part of a diagnostic test kit for identifying cells or tissue which misexpress a OST protein, such as by measuring a level of a OST-encoding nucleic acid in a sample of cells from a subject e.g., detecting OST MRNA levels or determining whether a genomic OST gene has been mutated or deleted.

“A polypeptide having a biologically active portion of OST” refers to polypeptides exhibiting activity similar, but not necessarily identical to, an activity of a polypeptide of the present invention, including mature forms, as measured in a particular biological assay, with or without dose dependency. A nucleic acid fragment encoding a “biologically active portion of OST” can be prepared by isolating a portion of OST 1-32, that encodes a polypeptide having a OST biological activity, expressing the encoded portion of OST protein (e.g., by recombinant expression in vitro) and assessing the activity of the encoded portion of OST. For example, a nucleic acid fragment encoding a biologically active portion of a OST polypeptide can optionally include an ATP-binding domain. In another embodiment, a nucleic acid fragment encoding a biologically active portion of OST includes one or more regions.

OST Variants

The invention further encompasses nucleic acid molecules that differ from the disclosed or referenced OST nucleotide sequences due to degeneracy of the genetic code. These nucleic acids thus encode the same OST protein as that encoded by nucleotide sequence comprising a OST nucleic acid as shown in, e.g., OST 1-32.

In addition to the rat OST nucleotide sequence shown in OST 1-32, it will be appreciated by those skilled in the art that DNA sequence polymorphisms that lead to changes in the amino acid sequences of a OST polypeptide may exist within a population (e.g., the human population). Such genetic polymorphism in the OST gene may exist among individuals within a population due to natural allelic variation. As used herein, the terms “gene” and “recombinant gene” refer to nucleic acid molecules comprising an open reading frame encoding a OST protein, preferably a mammalian OST protein. Such natural allelic variations can typically result in 1-5% variance in the nucleotide sequence of the OST gene. Any and all such nucleotide variations and resulting amino acid polymorphisms in OST that are the result of natural allelic variation and that do not alter the functional activity of OST are intended to be within the scope of the invention.

Moreover, nucleic acid molecules encoding OST proteins from other species, and thus that have a nucleotide sequence that differs from the human sequence of OST 1-32, are intended to be within the scope of the invention. Nucleic acid molecules corresponding to natural allelic variants and homologues of the OST DNAs of the invention can be isolated based on their homology to the human OST nucleic acids disclosed herein using the human cDNAs, or a portion thereof, as a hybridization probe according to standard hybridization techniques under stringent hybridization conditions. For example, a soluble human OST DNA can be isolated based on its homology to human membrane-bound OST. Likewise, a membrane-bound human OST DNA can be isolated based on its homology to soluble human OST.

Accordingly, in another embodiment, an isolated nucleic acid molecule of the invention is at least 6 nucleotides in length and hybridizes under stringent conditions to the nucleic acid molecule comprising the nucleotide sequence of OST 1-32. In another embodiment, the nucleic acid is at least 10, 25, 50, 100, 250 or 500 nucleotides in length. In another embodiment, an isolated nucleic acid molecule of the invention hybridizes to the coding region. As used herein, the term “hybridizes under stringent conditions” is intended to describe conditions for hybridization and washing under which nucleotide sequences at least 60% homologous to each other typically remain hybridized to each other.

Homologs (i.e., nucleic acids encoding OST proteins derived from species other than human) or other related sequences (e.g., paralogs) can be obtained by low, moderate or high stringency hybridization with all or a portion of the particular human sequence as a probe using methods well known in the art for nucleic acid hybridization and cloning.

As used herein, the phrase “stringent hybridization conditions” refers to conditions under which a probe, primer or oligonucleotide will hybridize to its target sequence, but to no other sequences. Stringent conditions are sequence-dependent and will be different in different circumstances. Longer sequences hybridize specifically at higher temperatures than shorter sequences. Generally, stringent conditions are selected to be about 5° C. lower than the thermal melting point (Tm) for the specific sequence at a defined ionic strength and pH. The Tm is the temperature (under defined ionic strength, pH and nucleic acid concentration) at which 50% of the probes complementary to the target sequence hybridize to the target sequence at equilibrium. Since the target sequences are generally present at excess, at Tm, 50% of the probes are occupied at equilibrium. Typically, stringent conditions will be those in which the salt concentration is less than about 1.0 M sodium ion, typically about 0.01 to 1.0 M sodium ion (or other salts) at pH 7.0 to 8.3 and the temperature is at least about 30° C. for short probes, primers or oligonucleotides (e.g., 10 nt to 50 nt) and at least about 60° C. for longer probes, primers and oligonucleotides. Stringent conditions may also be achieved with the addition of destabilizing agents, such as formamide.

Stringent conditions are known to those skilled in the art and can be found in CURRENT PROTOCOLS INMOLECULARBIOLOGY, John Wiley & Sons, N.Y. (1989), 6.3.1-6.3.6. Preferably, the conditions are such that sequences at least about 65%, 70%, 75%, 85%, 90%, 95%, 98%, or 99% homologous to each other typically remain hybridized to each other. A non-limiting example of stringent hybridization conditions is hybridization in a high salt buffer comprising 6× SSC, 50 mM Tris-HCl (pH 7.5), 1 mM EDTA, 0.02% PVP, 0.02% Ficoll, 0.02% BSA, and 500 mg/ml denatured salmon sperm DNA at 65° C. This hybridization is followed by one or more washes in 0.2× SSC, 0.01% BSA at 50° C. An isolated nucleic acid molecule of the invention that hybridizes under stringent conditions to the sequence of OST 1-32 corresponds to a naturally occurring nucleic acid molecule. As used herein, a “naturally-occurring” nucleic acid molecule refers to an RNA or DNA molecule having a nucleotide sequence that occurs in nature (e.g., encodes a natural protein).

In a second embodiment, a nucleic acid sequence that is hybridizable to the nucleic acid molecule comprising the nucleotide sequence of OST 1-32 or fragments, analogs or derivatives thereof, under conditions of moderate stringency is provided. A non-limiting example of moderate stringency hybridization conditions are hybridization in 6× SSC, 5× Denhardt's solution, 0.5% SDS and 100 mg/ml denatured salmon sperm DNA at 55° C., followed by one or more washes in 1× SSC, 0.1% SDS at 37° C. Other conditions of moderate stringency that may be used are well known in the art. See, e.g., Ausubel et al. (eds.), 1993, CURRENT PROTOCOLS IN MOLECULAR BIOLOGY, John Wiley & Sons, NY, and Kriegler, 1990, GENE TRANSFER AND EXPRESSION, A LABORATORY MANUAL, Stockton Press, New York.

In a third embodiment, a nucleic acid that is hybridizable to the nucleic acid molecule comprising the nucleotide sequence of OST 1-32 or fragments, analogs or derivatives thereof, under conditions of low stringency, is provided. A non-limiting example of low stringency hybridization conditions are hybridization in 35% formamide, 5× SSC, 50 mM Tris-HCl (pH 7.5), 5 mM EDTA, 0.02% PVP, 0.02% Ficoll, 0.2% BSA, 100 mg/ml denatured salmon sperm DNA, 10% (wt/vol) dextran sulfate at 40° C., followed by one or more washes in 2× SSC, 25 mM Tris-HCl (pH 7.4), 5 mM EDTA, and 0.1% SDS at 50° C. Other conditions of low stringency that may be used are well known in the art (e.g., as employed for cross-species hybridizations). See, e.g., Ausubel et al. (eds.), 1993, CURRENT PROTOCOLS IN MOLECULAR BIOLOGY, John Wiley & Sons, New York, and Kriegler, 1990, GENE TRANSFER AND EXPRESSION, A LABORATORY MANUAL, Stockton Press, New York; Shilo et al., 1981, Proc Natl Acad Sci USA 78: 6789-6792.

Conservative Mutations

In addition to naturally-occurring allelic variants of the OST sequence that may exist in the population, the skilled artisan will further appreciate that changes can be introduced into an OST nucleic acid or directly into an OST polypeptide sequence without altering the functional ability of the OST protein. In some embodiments, the nucleotide sequence of OST 1-32 will be altered, thereby leading to changes in the amino acid sequence of the encoded OST protein. For example, nucleotide substitutions that result in amino acid substitutions at various “non-essential” amino acid residues can be made in the sequence of OST 1-32 A “non-essential” amino acid residue is a residue that can be altered from the wild-type sequence of OST without altering the biological activity, whereas an “essential” amino acid residue is required for biological activity. For example, amino acid residues that are conserved among the OST proteins of the present invention, are predicted to be particularly unamenable to alteration.

In addition, amino acid residues that are conserved among family members of the OST proteins of the present invention, are also predicted to be particularly unamenable to alteration. As such, these conserved domains are not likely to be amenable to mutation. Other amino acid residues, however, (e.g., those that are not conserved or only semi-conserved among members of the OST proteins) may not be essential for activity and thus are likely to be amenable to alteration.

Another aspect of the invention pertains to nucleic acid molecules encoding OST proteins that contain changes in amino acid residues that are not essential for activity. Such OST proteins differ in amino acid sequence from the amino acid sequences of polypeptides encoded by nucleic acids containing OST 1-32, yet retain biological activity. In one embodiment, the isolated nucleic acid molecule comprises a nucleotide sequence encoding a protein, wherein the protein comprises an amino acid sequence at least about 45% homologous, more preferably 60%, and still more preferably at least about 70%, 80%, 90%, 95%, 98%, and most preferably at least about 99% homologous to the amino acid sequence of the amino acid sequences of polypeptides encoded by nucleic acids comprising OST 1-32.

An isolated nucleic acid molecule encoding a OST protein homologous to can be created by introducing one or more nucleotide substitutions, additions or deletions into the nucleotide sequence of a nucleic acid comprising OST 1-32, such that one or more amino acid substitutions, additions or deletions are introduced into the encoded protein.

Mutations can be introduced into a nucleic acid comprising OST 1-32 by standard techniques, such as site-directed mutagenesis and PCR-mediated mutagenesis. Preferably, conservative amino acid substitutions are made at one or more predicted non-essential amino acid residues. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a predicted nonessential amino acid residue in OST is replaced with another amino acid residue from the same side chain family. Alternatively, in another embodiment, mutations can be introduced randomly along all or part of a OST coding sequence, such as by saturation mutagenesis, and the resultant mutants can be screened for OST biological activity to identify mutants that retain activity. Following mutagenesis of the nucleic acid, the encoded protein can be expressed by any recombinant technology known in the art and the activity of the protein can be determined.

In one embodiment, a mutant OST protein can be assayed for (1) the ability to form protein:protein interactions with other OST proteins, other cell-surface proteins, or biologically active portions thereof, (2) complex formation between a mutant OST protein and a OST ligand; (3) the ability of a mutant OST protein to bind to an intracellular target protein or biologically active portion thereof; (e.g., avidin proteins); (4) the ability to bind ATP; or (5) the ability to specifically bind a OST protein antibody.

In other embodiment, the fragment of the complementary polynucleotide sequence of OST 1-32 wherein the fragment of the complementary polynucleotide sequence hybridizes to the first sequence.

In other specific embodiments, the nucleic acid is RNA or DNA. The fragment or the fragment of the complementary polynucleotide sequence of OST 1-32, wherein the fragment is between about 10 and about 100 nucleotides in length, e.g., between about 10 and about 90 nucleotides in length, or about 10 and about 75 nucleotides in length, about 10 and about 50 bases in length, about 10 and about 40 bases in length, or about 15 and about 30 bases in length.

Antisense

Another aspect of the invention pertains to isolated antisense nucleic acid molecules that are hybridizable to or complementary to the nucleic acid molecule comprising the nucleotide sequence of a OST sequence or fragments, analogs or derivatives thereof. An “antisense” nucleic acid comprises a nucleotide sequence that is complementary to a “sense” nucleic acid encoding a protein, e.g., complementary to the coding strand of a double-stranded cDNA molecule or complementary to an mRNA sequence. In specific aspects, antisense nucleic acid molecules are provided that comprise a sequence complementary to at least about 10, 25, 50, 100, 250 or 500 nucleotides or an entire OST coding strand, or to only a portion thereof. Nucleic acid molecules encoding fragments, homologs, derivatives and analogs of a OST protein, or antisense nucleic acids complementary to a nucleic acid comprising a OST nucleic acid sequence are additionally provided.

In one embodiment, an antisense nucleic acid molecule is antisense to a “coding region” of the coding strand of a nucleotide sequence encoding OST. The term “coding region” refers to the region of the nucleotide sequence comprising codons which are translated into amino acid residues. In another embodiment, the antisense nucleic acid molecule is antisense to a “noncoding region” of the coding strand of a nucleotide sequence encoding OST. The term “noncoding region” refers to 5′ and 3′ sequences which flank the coding region that are not translated into amino acids (i.e., also referred to as 5′ and 3′ untranslated regions).

Given the coding strand sequences encoding OST disclosed herein, antisense nucleic acids of the invention can be designed according to the rules of Watson and Crick or Hoogsteen base pairing. The antisense nucleic acid molecule can be complementary to the entire coding region of OST mRNA, but more preferably is an oligonucleotide that is antisense to only a portion of the coding or noncoding region of OST MRNA. For example, the antisense oligonucleotide can be complementary to the region surrounding the translation start site of OST mRNA. An antisense oligonucleotide can be, for example, about 5, 10, 15, 20, 25, 30, 35, 40, 45 or 50 nucleotides in length. An antisense nucleic acid of the invention can be constructed using chemical synthesis or enzymatic ligation reactions using procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used.

Examples of modified nucleotides that can be used to generate the antisense nucleic acid include: 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest, described further in the following subsection).

The antisense nucleic acid molecules of the invention are typically administered to a subject or generated in situ such that they hybridize with or bind to cellular MRNA and/or genomic DNA encoding a OST protein to thereby inhibit expression of the protein, e.g., by inhibiting transcription and/or translation. The hybridization can be by conventional nucleotide complementarity to form a stable duplex, or, for example, in the case of an antisense nucleic acid molecule that binds to DNA duplexes, through specific interactions in the major groove of the double helix. An example of a route of administration of antisense nucleic acid molecules of the invention includes direct injection at a tissue site. Alternatively, antisense nucleic acid molecules can be modified to target selected cells and then administered systemically. For example, for systemic administration, antisense molecules can be modified such that they specifically bind to receptors or antigens expressed on a selected cell surface, e.g., by linking the antisense nucleic acid molecules to peptides or antibodies that bind to cell surface receptors or antigens. The antisense nucleic acid molecules can also be delivered to cells using the vectors described herein. To achieve sufficient intracellular concentrations of antisense molecules, vector constructs in which the antisense nucleic acid molecule is placed under the control of a strong pol II or pol III promoter are preferred.

In yet another embodiment, the antisense nucleic acid molecule of the invention is an α-anomeric nucleic acid molecule. An α-anomeric nucleic acid molecule forms specific double-stranded hybrids with complementary RNA in which, contrary to the usual β-units, the strands run parallel to each other (Gaultier et al. (1987) Nucleic Acids Res 15: 6625-6641). The antisense nucleic acid molecule can also comprise a 2′-o-methylribonucleotide (Inoue et al. (1987) Nucleic Acids Res 15: 6131-6148) or a chimeric RNA-DNA analogue (Inoue et al. (1987) FEBS Lett 215: 327-330).

Ribozymes and PNA Moieties

In still another embodiment, an antisense nucleic acid of the invention is a ribozyme. Ribozymes are catalytic RNA molecules with ribonuclease activity that are capable of cleaving a single-stranded nucleic acid, such as an MRNA, to which they have a complementary region. Thus, ribozymes (e.g., hammerhead ribozymes (described in Haselhoff and Gerlach (1988) Nature 334:585-591)) can be used to catalytically cleave OST mRNA transcripts to thereby inhibit translation of OST mRNA. A ribozyme having specificity for a OST-encoding nucleic acid can be designed based upon the nucleotide sequence of a OST DNA disclosed herein. For example, a derivative of a Tetrahymena L-19 IVS RNA can be constructed in which the nucleotide sequence of the active site is complementary to the nucleotide sequence to be cleaved in a OST-encoding mRNA. See, e.g., Cech et al. U.S. Pat. No. 4,987,071; and Cech et al. U.S. Pat. No. 5,116,742. Alternatively, OST mRNA can be used to select a catalytic RNA having a specific ribonuclease activity from a pool of RNA molecules. See, e.g., Bartel et al., (1993) Science 261:1411-1418.

Alternatively, OST gene expression can be inhibited by targeting nucleotide sequences complementary to the regulatory region of a OST nucleic acid (e.g., the OST promoter and/or enhancers) to form triple helical structures that prevent transcription of the OST gene in target cells. See generally, Helene. (1991) Anticancer Drug Des. 6: 569-84; Helene. et al. (1992) Ann. N.Y. Acad. Sci. 660:27-36; and Maher (1992) Bioassays 14: 807-15.

In various embodiments, the nucleic acids of OST can be modified at the base moiety, sugar moiety or phosphate backbone to improve, e.g., the stability, hybridization, or solubility of the molecule. For example, the deoxyribose phosphate backbone of the nucleic acids can be modified to generate peptide nucleic acids (see Hyrup et al. (1996) Bioorg Med Chem 4: 5-23). As used herein, the terms “peptide nucleic acids” or “PNAs” refer to nucleic acid mimics, e.g., DNA mimics, in which the deoxyribose phosphate backbone is replaced by a pseudopeptide backbone and only the four natural nucleobases are retained. The neutral backbone of PNAs has been shown to allow for specific hybridization to DNA and RNA under conditions of low ionic strength. The synthesis of PNA oligomers can be performed using standard solid phase peptide synthesis protocols as described in Hyrup et al. (1996) above; Perry-O'Keefe et al. (1996) PNAS 93: 14670-675.

PNAs of OST can be used in therapeutic and diagnostic applications. For example, PNAs can be used as antisense or antigene agents for sequence-specific modulation of gene expression by, e.g., inducing transcription or translation arrest or inhibiting replication. PNAs of OST can also be used, e.g., in the analysis of single base pair mutations in a gene by, e.g., PNA directed PCR clamping; as artificial restriction enzymes when used in combination with other enzymes, e.g., S 1 nucleases (Hyrup B. (1996) above); or as probes or primers for DNA sequence and hybridization (Hyrup et al. (1996), above; Perry-O'Keefe (1996), above).

In another embodiment, PNAs of OST can be modified, e.g., to enhance their stability or cellular uptake, by attaching lipophilic or other helper groups to PNA, by the formation of PNA-DNA chimeras, or by the use of liposomes or other techniques of drug delivery known in the art. For example, PNA-DNA chimeras of OST can be generated that may combine the advantageous properties of PNA and DNA. Such chimeras allow DNA recognition enzymes, e.g., RNase H and DNA polymerases, to interact with the DNA portion while the PNA portion would provide high binding affinity and specificity. PNA-DNA chimeras can be linked using linkers of appropriate lengths selected in terms of base stacking, number of bonds between the nucleobases, and orientation (Hyrup (1996) above). The synthesis of PNA-DNA chimeras can be performed as described in Hyrup (1996) above and Finn et al. (1996) Nucl Acids Res 24: 3357-63. For example, a DNA chain can be synthesized on a solid support using standard phosphoramidite coupling chemistry, and modified nucleoside analogs, e.g., 5′-(4-methoxytrityl)amino-5′-deoxy-thymidine phosphoramidite, can be used between the PNA and the 5′ end of DNA (Mag et al. (1989) Nucl Acid Res 17: 5973-88). PNA monomers are then coupled in a stepwise manner to produce a chimeric molecule with a 5′ PNA segment and a 3′ DNA segment (Finn et al. (1996) above). Alternatively, chimeric molecules can be synthesized with a 5′ DNA segment and a 3′ PNA segment. See, Petersen et al. (1975) Bioorg Med Chem Lett 5: 1119-11124.

In other embodiments, the oligonucleotide may include other appended groups such as peptides (e.g., for targeting host cell receptors in vivo), or agents facilitating transport across the cell membrane (see, e.g., Letsinger et al., 1989, Proc. Natl. Acad. Sci. U.S.A. 86:6553-6556; Lemaitre et al., 1987, Proc. Natl. Acad. Sci. 84:648-652; PCT Publication No. WO88/09810) or the blood-brain barrier (see, e.g., PCT Publication No. WO89/10134). In addition, oligonucleotides can be modified with hybridization triggered cleavage agents (See, e.g., Krol et al., 1988, BioTechniques 6:958-976) or intercalating agents. (See, e.g., Zon, 1988, Pharm. Res. 5: 539-549). To this end, the oligonucleotide may be conjugated to another molecule, e.g., a peptide, a hybridization triggered cross-linking agent, a transport agent, a hybridization-triggered cleavage agent, etc.

OST Polypeptides

One aspect of the invention pertains to isolated OST proteins, e.g., OST 1-32 (SEQ ID NO:33-53), and biologically active portions thereof, or derivatives, fragments, analogs or homologs thereof. Also provided are polypeptide fragments suitable for use as immunogens to raise anti-OST antibodies. In one embodiment, native OST proteins can be isolated from cells or tissue sources by an appropriate purification scheme using standard protein purification techniques. In another embodiment, OST proteins are produced by recombinant DNA techniques. Alternative to recombinant expression, a OST protein or polypeptide can be synthesized chemically using standard peptide synthesis techniques.

An “isolated” or “purified” protein or biologically active portion thereof is substantially free of cellular material or other contaminating proteins from the cell or tissue source from which the OST protein is derived, or substantially free from chemical precursors or other chemicals when chemically synthesized. The language “substantially free of cellular material” includes preparations of OST protein in which the protein is separated from cellular components of the cells from which it is isolated or recombinantly produced. In one embodiment, the language “substantially free of cellular material” includes preparations of OST protein having less than about 30% (by dry weight) of non-OST protein (also referred to herein as a “contaminating protein”), more preferably less than about 20% of non-OST protein, still more preferably less than about 10% of non-OST protein, and most preferably less than about 5% non-OST protein. When the OST protein or biologically active portion thereof is recombinantly produced, it is also preferably substantially free of culture medium, i.e., culture medium represents less than about 20%, more preferably less than about 10%, and most preferably less than about 5% of the volume of the protein preparation.

The language “substantially free of chemical precursors or other chemicals” includes preparations of OST protein in which the protein is separated from chemical precursors or other chemicals that are involved in the synthesis of the protein. In one embodiment, the language “substantially free of chemical precursors or other chemicals” includes preparations of OST protein having less than about 30% (by dry weight) of chemical precursors or non-OST chemicals, more preferably less than about 20% chemical precursors or non-OST chemicals, still more preferably less than about 10% chemical precursors or non-OST chemicals, and most preferably less than about 5% chemical precursors or non-OST chemicals.

Biologically active portions of a OST protein include peptides comprising amino acid sequences sufficiently homologous to or derived from the amino acid sequence of the OST protein, e.g., the amino acid sequence encoded by a nucleic acid comprising OST 1-32 that include fewer amino acids than the full length OST proteins, and exhibit at least one activity of a OST protein. Typically, biologically active portions comprise a domain or motif with at least one activity of the OST protein. A biologically active portion of a OST protein can be a polypeptide which is, for example, 10, 25, 50, 100 or more amino acids in length.

A biologically active portion of a OST protein of the present invention may contain at least one of the above-identified domains conserved between the OST proteins. An alternative biologically active portion of a OST protein may contain at least two of the above-identified domains. Another biologically active portion of a OST protein may contain at least three of the above-identified domains. Yet another biologically active portion of a OST protein of the present invention may contain at least four of the above-identified domains.

Moreover, other biologically active portions, in which other regions of the protein are deleted, can be prepared by recombinant techniques and evaluated for one or more of the functional activities of a native OST protein.

In some embodiments, the OST protein is substantially homologous to one of these OST proteins and retains its the functional activity, yet differs in amino acid sequence due to natural allelic variation or mutagenesis, as described in detail below.

Determining Homology Between Two or More Sequences

To determine the percent homology of two amino acid sequences or of two nucleic acids, the sequences are aligned for optimal comparison purposes (e.g., gaps can be introduced in the sequence of a first amino acid or nucleic acid sequence for optimal alignment with a second amino or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are homologous at that position (i.e., as used herein amino acid or nucleic acid “homology” is equivalent to amino acid or nucleic acid “identity”).

The nucleic acid sequence homology may be determined as the degree of identity between two sequences. The homology may be determined using computer programs known in the art, such as GAP software provided in the GCG program package. See Needleman and Wunsch 1970 J Mol Biol 48: 443-453. Using GCG GAP software with the following settings for nucleic acid sequence comparison: GAP creation penalty of 5.0 and GAP extension penalty of 0.3, the coding region of the analogous nucleic acid sequences referred to above exhibits a degree of identity preferably of at least 70%, 75%, 80%, 85%, 90%, 95%, 98%, or 99%, with the CDS (encoding) part of a DNA sequence comprising OST: 1-32.

The term “sequence identity” refers to the degree to which two polynucleotide or polypeptide sequences are identical on a residue-by-residue basis over a particular region of comparison. The term “percentage of sequence identity” is calculated by comparing two optimally aligned sequences over that region of comparison, determining the number of positions at which the identical nucleic acid base (e.g., A, T, C, G, U, or I, in the case of nucleic acids) occurs in both sequences to yield the number of matched positions, dividing the number of matched positions by the total number of positions in the region of comparison (i.e., the window size), and multiplying the result by 100 to yield the percentage of sequence identity. The term “substantial identity” as used herein denotes a characteristic of a polynucleotide sequence, wherein the polynucleotide comprises a sequence that has at least 80 percent sequence identity, preferably at least 85 percent identity and often 90 to 95 percent sequence identity, more usually at least 99 percent sequence identity as compared to a reference sequence over a comparison region.

Chimeric and Fusion Proteins

The invention also provides OST chimeric or fusion proteins. As used herein, an OST “chimeric protein” or “fusion protein” comprises an OST polypeptide operatively linked to a non-OST polypeptide. A “OST polypeptide” refers to a polypeptide having an amino acid sequence corresponding to OST, whereas a “non-OST polypeptide” refers to a polypeptide having an amino acid sequence corresponding to a protein that is not substantially homologous to the OST protein, e.g., a protein that is different from the OST protein and that is derived from the same or a different organism. Within an OST fusion protein the OST polypeptide can correspond to all or a portion of an OST protein. In one embodiment, an OST fusion protein comprises at least one biologically active portion of an OST protein. In another embodiment, an OST fusion protein comprises at least two biologically active portions of an OST protein. In yet another embodiment, an OST fusion protein comprises at least three biologically active portions of an OST protein. Within the fusion protein, the term “operatively linked” is intended to indicate that the OST polypeptide and the non-OST polypeptide are fused in-frame to each other. The non-OST polypeptide can be fused to the N-terminus or C-terminus of the OST polypeptide.

For example, in one embodiment an OST fusion protein comprises an OST domain operably linked to the extracellular domain of a second protein. Such fusion proteins can be further utilized in screening assays for compounds which modulate OST activity (such assays are described in detail below).

In yet another embodiment, the fusion protein is a GST-OST fusion protein in which the OST sequences are fused to the C-terminus of the GST (i.e., glutathione S-transferase) sequences. Such fusion proteins can facilitate the purification of recombinant OST.

In another embodiment, the fusion protein is an OST protein containing a heterologous signal sequence at its N-terminus. For example, a native OST signal sequence can be removed and replaced with a signal sequence from another protein. In certain host cells (e.g., mammalian host cells), expression and/or secretion of OST can be increased through use of a heterologous signal sequence.

In yet another embodiment, the fusion protein is an OST-immunoglobulin fusion protein in which the OST sequences comprising one or more domains are fused to sequences derived from a member of the immunoglobulin protein family. The OST-immunoglobulin fusion proteins of the invention can be incorporated into pharmaceutical compositions and administered to a subject to inhibit an interaction between a OST ligand and a OST protein on the surface of a cell, to thereby suppress OST-mediated signal transduction in vivo. The OST-immunoglobulin fusion proteins can be used to affect the bioavailability of an OST cognate ligand. Inhibition of the OST ligand/OST interaction may be useful therapeutically for both the treatments of proliferative and differentiative disorders, as well as modulating (e.g. promoting or inhibiting) cell survival. Moreover, the OST-immunoglobulin fusion proteins of the invention can be used as immunogens to produce anti-OST antibodies in a subject, to purify OST ligands, and in screening assays to identify molecules that inhibit the interaction of OST with a OST ligand.

An OST chimeric or fusion protein of the invention can be produced by standard recombinant DNA techniques. For example, DNA fragments coding for the different polypeptide sequences are ligated together in-frame in accordance with conventional techniques, e.g., by employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers that give rise to complementary overhangs between two consecutive gene fragments that can subsequently be annealed and reamplified to generate a chimeric gene sequence (see, for example, Ausubel et al. (eds.) CURRENT PROTOCOLS IN MOLECULAR BIOLOGY, John Wiley & Sons, 1992). Moreover, many expression vectors are commercially available that already encode a fusion moiety (e.g., a GST polypeptide). An OST-encoding nucleic acid can be cloned into such an expression vector such that the fusion moiety is linked in-frame to the OST protein.

OST Agonists and Antagonists

The present invention also pertains to variants of the OST proteins that function as either OST agonists (mimetics) or as OST antagonists. Variants of the OST protein can be generated by mutagenesis, e.g., discrete point mutation or truncation of the OST protein. An agonist of the OST protein can retain substantially the same, or a subset of, the biological activities of the naturally occurring form of the OST protein. An antagonist of the OST protein can inhibit one or more of the activities of the naturally occurring form of the OST protein by, for example, competitively binding to a downstream or upstream member of a cellular signaling cascade which includes the OST protein. Thus, specific biological effects can be elicited by treatment with a variant of limited function. In one embodiment, treatment of a subject with a variant having a subset of the biological activities of the naturally occurring form of the protein has fewer side effects in a subject relative to treatment with the naturally occurring form of the OST proteins.

Variants of the OST protein that function as either OST agonists (mimetics) or as OST antagonists can be identified by screening combinatorial libraries of mutants, e.g., truncation mutants, of the OST protein for OST protein agonist or antagonist activity. In one embodiment, a variegated library of OST variants is generated by combinatorial mutagenesis at the nucleic acid level and is encoded by a variegated gene library. A variegated library of OST variants can be produced by, for example, enzymatically ligating a mixture of synthetic oligonucleotides into gene sequences such that a degenerate set of potential OST sequences is expressible as individual polypeptides, or alternatively, as a set of larger fusion proteins (e.g., for phage display) containing the set of OST sequences therein. There are a variety of methods which can be used to produce libraries of potential OST variants from a degenerate oligonucleotide sequence. Chemical synthesis of a degenerate gene sequence can be performed in an automatic DNA synthesizer, and the synthetic gene then ligated into an appropriate expression vector. Use of a degenerate set of genes allows for the provision, in one mixture, of all of the sequences encoding the desired set of potential OST sequences. Methods for synthesizing degenerate oligonucleotides are known in the art (see, e.g., Narang (1983) Tetrahedron 39:3; Itakura et al. (1984) Annu Rev Biochem 53:323; Itakura et al. (1984) Science 198:1056; Ike et al. (1983) Nucl Acid Res 11:477.

Polypeptide Libraries

In addition, libraries of fragments of the OST protein coding sequence can be used to generate a variegated population of OST fragments for screening and subsequent selection of variants of an OST protein. In one embodiment, a library of coding sequence fragments can be generated by treating a double stranded PCR fragment of a OST coding sequence with a nuclease under conditions wherein nicking occurs only about once per molecule, denaturing the double stranded DNA, renaturing the DNA to form double stranded DNA that can include sense/antisense pairs from different nicked products, removing single stranded portions from reformed duplexes by treatment with S1 nuclease, and ligating the resulting fragment library into an expression vector. By this method, an expression library can be derived which encodes N-terminal and internal fragments of various sizes of the OST protein.

Several techniques are known in the art for screening gene products of combinatorial libraries made by point mutations or truncation, and for screening cDNA libraries for gene products having a selected property. Such techniques are adaptable for rapid screening of the gene libraries generated by the combinatorial mutagenesis of OST proteins. The most widely used techniques, which are amenable to high throughput analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected. Recursive ensemble mutagenesis (REM), a new technique that enhances the frequency of functional mutants in the libraries, can be used in combination with the screening assays to identify OST variants (Arkin and Yourvan (1992) PNAS 89:7811-7815; Delgrave et al. (1993) Protein Engineering 6:327-331).

Anti-OST Antibodies

An isolated OST protein, or a portion or fragment thereof, can be used as an immunogen to generate antibodies that bind OST using standard techniques for polyclonal and monoclonal antibody preparation. The full-length OST protein can be used or, alternatively, the invention provides antigenic peptide fragments of OST for use as immunogens. The antigenic peptide of OST comprises at least 8 amino acid residues of the amino acid sequence encoded by a nucleic acid comprising the nucleic acid sequence shown in OST 1-32 and encompasses an epitope of OST such that an antibody raised against the peptide forms a specific immune complex with OST. Preferably, the antigenic peptide comprises at least 10 amino acid residues, more preferably at least 15 amino acid residues, even more preferably at least 20 amino acid residues, and most preferably at least 30 amino acid residues. Preferred epitopes encompassed by the antigenic peptide are regions of OST that are located on the surface of the protein, e.g., hydrophilic regions. As a means for targeting antibody production, hydropathy plots showing regions of hydrophilicity and hydrophobicity may be generated by any method well known in the art, including, for example, the Kyte Doolittle or the Hopp Woods methods, either with or without Fourier transformation. See, e.g., Hopp and Woods, 1981, Proc. Nat. Acad. Sci. USA 78: 3824-3828; Kyte and Doolittle 1982, J. Mol. Biol. 157: 105-142, each incorporated herein by reference in their entirety.

OST polypeptides or derivatives, fragments, analogs or homologs thereof, may be utilized as immunogens in the generation of antibodies that immunospecifically-bind these protein components. The term “antibody” as used herein refers to immunoglobulin molecules and immunologically active portions of immunoglobulin molecules, i.e., molecules that contain an antigen binding site that specifically binds (immunoreacts with) an antigen. Such antibodies include, but are not limited to, polyclonal, monoclonal, chimeric, single chain, F_{ab} and F_{(ab′)2} fragments, and an F_{ab} expression library. Various procedures known within the art may be used for the production of polyclonal or monoclonal antibodies to an OST protein sequence, or derivatives, fragments, analogs or homologs thereof. Some of these proteins are discussed below.

For the production of polyclonal antibodies, various suitable host animals (e.g., rabbit, goat, mouse or other mammal) may be immunized by injection with the native protein, or a synthetic variant thereof, or a derivative of the foregoing. An appropriate immunogenic preparation can contain, for example, recombinantly expressed OST protein or a chemically synthesized OST polypeptide. The preparation can further include an adjuvant. Various adjuvants used to increase the immunological response include, but are not limited to, Freund's (complete and incomplete), mineral gels (e.g., aluminum hydroxide), surface active substances (e.g., lysolecithin, pluronic polyols, polyanions, peptides, oil emulsions, dinitrophenol, etc.), human adjuvants such as Bacille Calmette-Guerin and Corynebacterium parvum, or similar immunostimulatory agents. If desired, the antibody molecules directed against OST can be isolated from the mammal (e.g., from the blood) and further purified by well known techniques, such as protein A chromatography to obtain the IgG fraction.

The term “monoclonal antibody” or “monoclonal antibody composition”, as used herein, refers to a population of antibody molecules that contain only one species of an antigen binding site capable of immunoreacting with a particular epitope of OST. A monoclonal antibody composition thus typically displays a single binding affinity for a particular OST protein with which it immunoreacts. For preparation of monoclonal antibodies directed towards a particular OST protein, or derivatives, fragments, analogs or homologs thereof, any technique that provides for the production of antibody molecules by continuous cell line culture may be utilized. Such techniques include, but are not limited to, the hybridoma technique (see Kohler & Milstein, 1975 Nature 256: 495-497); the trioma technique; the human B-cell hybridoma technique (see Kozbor, et al., 1983 Immunol Today 4: 72) and the EBV hybridoma technique to produce human monoclonal antibodies (see Cole, et al., 1985 In: MONOCLONAL ANTIBODIES AND CANCER THERAPY, Alan R. Liss, Inc., pp. 77-96). Human monoclonal antibodies may be utilized in the practice of the present invention and may be produced by using human hybridomas (see Cote, et al., 1983. Proc Natl Acad Sci USA 80: 2026-2030) or by transforming human B-cells with Epstein Barr Virus in vitro (see Cole, et al., 1985 In: MONOCLONAL ANTIBODIES AND CANCER THERAPY, Alan R. Liss, Inc., pp. 77-96).

According to the invention, techniques can be adapted for the production of single-chain antibodies specific to a OST protein (see e.g., U.S. Pat. No. 4,946,778). In addition, methods can be adapted for the construction of F_{ab} expression libraries (see e.g., Huse, et al., 1989 Science 246: 1275-1281) to allow rapid and effective identification of monoclonal F_{ab} fragments with the desired specificity for a OST protein or derivatives, fragments, analogs or homologs thereof. Non-human antibodies can be “humanized” by techniques well known in the art. See e.g., U.S. Pat. No. 5,225,539. Antibody fragments that contain the idiotypes to a OST protein may be produced by techniques known in the art including, but not limited to: (i) an F_{(ab′)2} fragment produced by pepsin digestion of an antibody molecule; (ii) an F_{ab} fragment generated by reducing the disulfide bridges of an F_{(ab′)2} fragment; (iii) an F_{ab} fragment generated by the treatment of the antibody molecule with papain and a reducing agent and (iv) Fᵥ fragments.

Additionally, recombinant anti-OST antibodies, such as chimeric and humanized monoclonal antibodies, comprising both human and non-human portions, which can be made using standard recombinant DNA techniques, are within the scope of the invention. Such chimeric and humanized monoclonal antibodies can be produced by recombinant DNA techniques known in the art, for example using methods described in PCT International Application No. PCT/US86/02269; European Patent Application No. 184,187; European Patent Application No. 171,496; European Patent Application No. 173,494; PCT International Publication No. WO 86/01533; U.S. Pat. No. 4,816,567; European Patent Application No. 125,023; Better et al.(1988) Science 240:1041-1043; Liu et al. (1987) PNAS 84:3439-3443; Liu et al. (1987) J Immunol. 139:3521-3526; Sun et al. (1987) PNAS 84:214-218; Nishimura et al. (1987) Cancer Res 47:999-1005; Wood et al. (1985) Nature 314:446-449; Shaw et al. (1988) J Natl Cancer Inst. 80:1553-1559); Morrison(1985) Science 229:1202-1207; Oi et al. (1986) BioTechniques 4:214; U.S. Pat. No. 5,225,539; Jones et al. (1986) Nature 321:552-525; Verhoeyan et al. (1988) Science 239:1534; and Beidler et al. (1988) J Immunol 141:4053-4060.

In one embodiment, methods for the screening of antibodies that possess the desired specificity include, but are not limited to, enzyme-linked immunosorbent assay (ELISA) and other immunologically-mediated techniques known within the art. In a specific embodiment, selection of antibodies that are specific to a particular domain of a OST protein is facilitated by generation of hybridomas that bind to the fragment of a OST protein possessing such a domain. Antibodies that are specific for one or more domains within a OST protein, e.g., domains spanning the above-identified conserved regions of OST family proteins, or derivatives, fragments, analogs or homologs thereof, are also provided herein.

Anti-OST antibodies may be used in methods known within the art relating to the localization and/or quantitation of a OST protein (e.g., for use in measuring levels of the OST protein within appropriate physiological samples, for use in diagnostic methods, for use in imaging the protein, and the like). In a given embodiment, antibodies for OST proteins, or derivatives, fragments, analogs or homologs thereof, that contain the antibody derived binding domain, are utilized as pharmacologically-active compounds [hereinafter “Therapeutics”].

An anti-OST antibody (e.g., monoclonal antibody) can be used to isolate OST by standard techniques, such as affinity chromatography or immunoprecipitation. An anti-OST antibody can facilitate the purification of natural OST from cells and of recombinantly produced OST expressed in host cells. Moreover, an anti-OST antibody can be used to detect OST protein (e.g., in a cellular lysate or cell supernatant) in order to evaluate the abundance and pattern of expression of the OST protein. Anti-OST antibodies can be used diagnostically to monitor protein levels in tissue as part of a clinical testing procedure, e.g., to, for example, determine the efficacy of a given treatment regimen. Detection can be facilitated by coupling (i.e., physically linking) the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H.

OST Recombinant Expression Vectors and Host Cells

Another aspect of the invention pertains to vectors, preferably expression vectors, containing a nucleic acid encoding OST protein, or derivatives, fragments, analogs or homologs thereof. As used herein, the term “vector” refers to a nucleic acid molecule capable of transporting another nucleic acid to which it has been linked. One type of vector is a “plasmid”, which refers to a linear or circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “expression vectors”. In general, expression vectors of utility in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

The recombinant expression vectors of the invention comprise a nucleic acid of the invention in a form suitable for expression of the nucleic acid in a host cell, which means that the recombinant expression vectors include one or more regulatory sequences, selected on the basis of the host cells to be used for expression, that is operatively linked to the nucleic acid sequence to be expressed. Within a recombinant expression vector, “operably linked” is intended to mean that the nucleotide sequence of interest is linked to the regulatory sequence(s) in a manner that allows for expression of the nucleotide sequence (e.g., in an in vitro transcription/translation system or in a host cell when the vector is introduced into the host cell). The term “regulatory sequence” is intended to includes promoters, enhancers and other expression control elements (e.g., polyadenylation signals). Such regulatory sequences are described, for example, in Goeddel; GENE EXPRESSION TECHNOLOGY: METHODS IN ENZYMOLOGY 185, Academic Press, San Diego, Calif. (1990). Regulatory sequences include those that direct constitutive expression of a nucleotide sequence in many types of host cell and those that direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, etc. The expression vectors of the invention can be introduced into host cells to thereby produce proteins or peptides, including fusion proteins or peptides, encoded by nucleic acids as described herein (e.g., OST proteins, mutant forms of OST, fusion proteins, etc.).

The recombinant expression vectors of the invention can be designed for expression of OST in prokaryotic or eukaryotic cells. For example, OST can be expressed in bacterial cells such as E. coli, insect cells (using baculovirus expression vectors) yeast cells or mammalian cells. Suitable host cells are discussed further in Goeddel, GENE EZPRESSION TECHNOLOGY: METHODS IN ENZYMOLOGY 185, Academic Press, San Diego, Calif. (1990). Alternatively, the recombinant expression vector can be transcribed and translated in vitro, for example using T7 promoter regulatory sequences and T7 polymerase.

Expression of proteins in prokaryotes is most often carried out in E. coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: (1) to increase expression of recombinant protein; (2) to increase the solubility of the recombinant protein; and (3) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include pGEX (Pharmacia Biotech Inc; Smith and Johnson (1988) Gene 67:31-40), pMAL (New England Biolabs, Beverly, Mass.) and pRIT5 (Pharmacia, Piscataway, N.J.) that fuse glutathione S-transferase (GST), maltose E binding protein, or protein A, respectively, to the target recombinant protein.

Examples of suitable inducible non-fusion E. coli expression vectors include pTrc (Amrann et al., (1988) Gene 69:301-315) and pET 11d (Studier et al., GENE EXPRESSION TECHNOLOGY: METHODS IN ENZYMOLOGY 185, Academic Press, San Diego, Calif. (1990) 60-89).

One strategy to maximize recombinant protein expression in E. coli is to express the protein in a host bacteria with an impaired capacity to proteolytically cleave the recombinant protein. See, Gottesman, GENE EXPRESSION TECHNOLOGY: METHODS IN ENZYMOLOGY 185, Academic Press, San Diego, Calif. (1990) 119-128. Another strategy is to alter the nucleic acid sequence of the nucleic acid to be inserted into an expression vector so that the individual codons for each amino acid are those preferentially utilized in E. coli (Wada et al., (1992) Nucleic Acids Res. 20:2111-2118). Such alteration of nucleic acid sequences of the invention can be carried out by standard DNA synthesis techniques.

In another embodiment, the OST expression vector is a yeast expression vector. Examples of vectors for expression in yeast S. cerevisiae include pYepSec1 (Baldari, et al., (1987) EMBO J 6:229-234), pMFa (Kurjan and Herskowitz, (1982) Cell 30:933-943), pJRY88 (Schultz et al., (1987) Gene 54:113-123), pYES2 (Invitrogen Corporation, San Diego, Calif.), and picZ (InVitrogen Corp, San Diego, Calif.).

Alternatively, OST can be expressed in insect cells using baculovirus expression vectors. Baculovirus vectors available for expression of proteins in cultured insect cells (e.g., SF9 cells) include the pAc series (Smith et al. (1983) Mol Cell Biol 3:2156-2165) and the pVL series (Lucklow and Summers (1989) Virology 170:31-39).

In yet another embodiment, a nucleic acid of the invention is expressed in mammalian cells using a mammalian expression vector. Examples of mammalian expression vectors include pCDM8 (Seed (1987) Nature 329:840) and pMT2PC (Kaufman et al. (1987) EMBO J 6: 187-195). When used in mammalian cells, the expression vector's control functions are often provided by viral regulatory elements. For example, commonly used promoters are derived from polyoma, Adenovirus 2, cytomegalovirus and Simian Virus 40. For other suitable expression systems for both prokaryotic and eukaryotic cells. See, e.g., Chapters 16 and 17 of Sambrook et al., MOLECULAR CLONING: A LABORATORY MANUAL. 2nd ed., Cold Spring Harbor Laboratory, Cold Spring Harbor Laboratory Press, Cold Spring Harbor, N.Y., 1989.

In another embodiment, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert et al. (1987) Genes Dev 1:268-277), lymphoid-specific promoters (Calame and Eaton (1988) Adv Immunol 43:235-275), in particular promoters of T cell receptors (Winoto and Baltimore (1989) EMBO J 8:729-733) and immunoglobulins (Banerji et al. (1983) Cell 33:729-740; Queen and Baltimore (1983) Cell 33:741-748), neuron-specific promoters (e.g., the neurofilament promoter; Byrne and Ruddle (1989) PNAS 86:5473-5477), pancreas-specific promoters (Edlund et al. (1985) Science 230:912-916), and mammary gland-specific promoters (e.g., milk whey promoter; U.S. Pat. No. 4,873,316 and European Application Publication No. 264,166). Developmentally-regulated promoters are also encompassed, e.g., the murine hox promoters (Kessel and Gruss (1990) Science 249:374-379) and the α-fetoprotein promoter (Campes and Tilghman (1989) Genes Dev 3:537-546).

The invention further provides a recombinant expression vector comprising a DNA molecule of the invention cloned into the expression vector in an antisense orientation. That is, the DNA molecule is operatively linked to a regulatory sequence in a manner that allows for expression (by transcription of the DNA molecule) of an RNA molecule that is antisense to OST mRNA. Regulatory sequences operatively linked to a nucleic acid cloned in the antisense orientation can be chosen that direct the continuous expression of the antisense RNA molecule in a variety of cell types, for instance viral promoters and/or enhancers, or regulatory sequences can be chosen that direct constitutive, tissue specific or cell type specific expression of antisense RNA. The antisense expression vector can be in the form of a recombinant plasmid, phagemid or attenuated virus in which antisense nucleic acids are produced under the control of a high efficiency regulatory region, the activity of which can be determined by the cell type into which the vector is introduced. For a discussion of the regulation of gene expression using antisense genes see Weintraub et al., “Antisense RNA as a molecular tool for genetic analysis,” Reviews—Trends in Genetics, Vol. 1(1) 1986.

Another aspect of the invention pertains to host cells into which a recombinant expression vector of the invention has been introduced. The terms “host cell” and “recombinant host cell” are used interchangeably herein. It is understood that such terms refer not only to the particular subject cell but also to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell. For example, OST protein can be expressed in bacterial cells such as E. coli, insect cells, yeast or mammalian cells (such as Chinese hamster ovary cells (CHO) or COS cells). Other suitable host cells are known to those skilled in the art.

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation. Suitable methods for transforming or transfecting host cells can be found in Sambrook, et al. (MOLECULAR CLONING: A LABORATORY MANUAL. 2nd ed., Cold Spring Harbor Laboratory, Cold Spring Harbor Laboratory Press, Cold Spring Harbor, N.Y., 1989), and other laboratory manuals.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., resistance to antibiotics) is generally introduced into the host cells along with the gene of interest. Various selectable markers include those that confer resistance to drugs, such as G418, hygromycin and methotrexate. Nucleic acid encoding a selectable marker can be introduced into a host cell on the same vector as that encoding OST or can be introduced on a separate vector. Cells stably transfected with the introduced nucleic acid can be identified by drug selection (e.g., cells that have incorporated the selectable marker gene will survive, while the other cells die).

A host cell of the invention, such as a prokaryotic or eukaryotic host cell in culture, can be used to produce (i.e., express) an OST protein. Accordingly, the invention further provides methods for producing OST protein using the host cells of the invention. In one embodiment, the method comprises culturing the host cell of invention (into which a recombinant expression vector encoding OST has been introduced) in a suitable medium such that OST protein is produced. In another embodiment, the method further comprises isolating OST from the medium or the host cell.

Kits and Nucleic Acid Collections for Identifying OST Nucleic Acids

In another aspect, the invention provides a kit useful for examining a bone disorder, identifying parathyroid hormone receptor ligands and osteoregenerative agents. The kit can include nucleic acids that detect two or more OST sequences. In preferred embodiments, the kit includes reagents which detect 3, 4, 5, 6, 8, 10, 12, 15, 20, 25, 30, 35, 40 or all of the OST nucleic acid sequences.

The invention also includes an isolated plurality of sequences which can identify one or more OST responsive nucleic acid sequences.

The kit or plurality may include, e.g., sequence homologous to OST nucleic acid sequences, or sequences which can specifically identify one or more OST nucleic acid sequences.

### EXAMPLES

### Example 1

### Data Analysis

To investigate gene expression changes over time in a complex system, a multivariate cluster analysis of the data set was employed. Bands that showed a 2-fold or more change in expression were included in the analysis (total 537 bands). Through the use of a Self-Organizing Map (SOM) algorithm executed over 100,000 reiterations, the 537 bands were partitioned into 10 clusters (SOMs 1-10). Cluster means were calculated and plotted as a function of time. The following strategy was used to identify and analyze differentially expressed genes:

Step 1:

Bands (representing gene fragments) that did not change expression levels in any one of the nine treatments (compared to the vehicle-treated group) were filtered out. Bands showing differential expression in at least one of the nine treatments by a factor greater than or equal to ±2-fold were included in the analysis.

Step 2:

Hierarchical clustering methods were applied first to help simultaneously visualize any underlying patterns in the expression profiles of all the bands included in the analysis. In Hierarchical Clustering, relationships among the bands are represented by a tree (dendrogram) whose branch length reflect the degree of similarity between the bands' expression profiles, as assessed by a pairwise similarity function. Following the clustering, the primary data set were re-ordered such that bands of high expression similarity are adjacent to each other.

Step 3:

Non-hierarchical clustering, specifically Self-Organizing Map (SOM) analysis, was performed to group the bands into a defined number of clusters with similar expression profiles across the nine treatments. The number of clusters were optimized by iterative analysis to minimize overlap between cluster properties. The Self-Organizing Map (SOM) method is useful to identify a small number of prominent classes within a data set. The user specifies the nubmer of clusters to be identified. The SOM algorithm then finds an optimal set of “cetners” in data-space around which the data points begin to aggregate. The data set is then partitioned, with each center defining a cluster consisting of the data points nearest to it.

Step 4:

The mean values for each cluster for each treatment were plotted to generate cluster expression profiles over the treatments. Ten cluster profiles were obtained.

Step 5:

Gene fragments were chosen for follow-up studies based on their membership in clusters with interesting expression patterns.

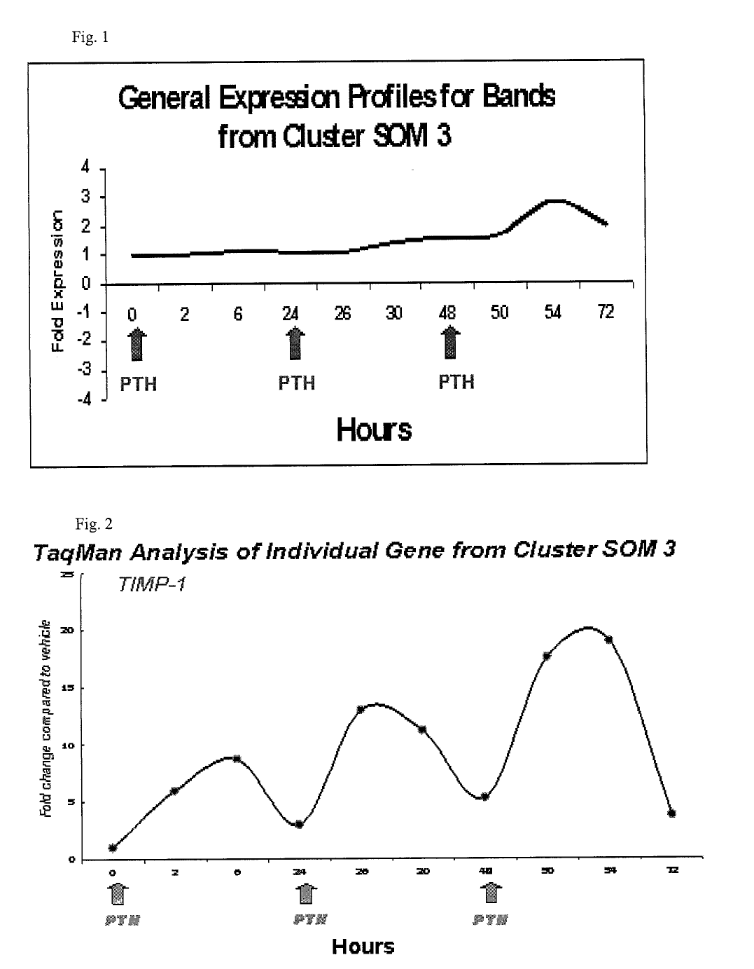

Of the 10 data clusters, three were distinctly interesting. Bands in SOM3 exhibited increased expression beginning at day 2, 24 hours and maintained increased expression at all subsequent time points (FIG. 1). In Cluster 3 (FIG. 1), 26 genes were identified, including bone sialoprotein, brain creatinine kinase, osteonectin, TIMP-1 (see FIG. 2), and six novel sequences.

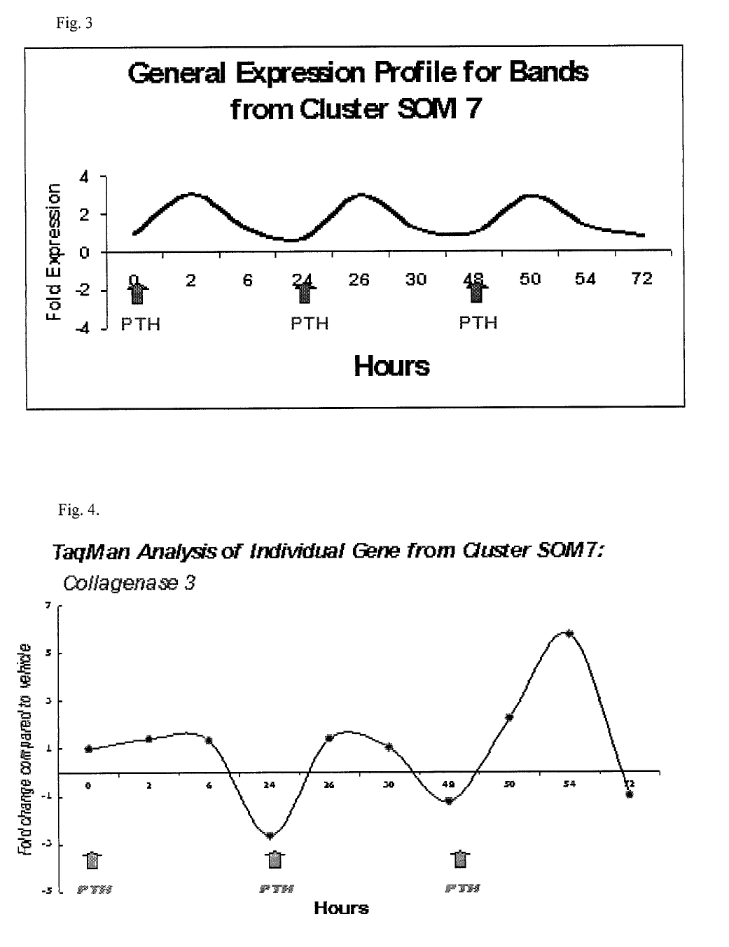

Bands in SOM7 exhibited rapid transient increases in expression (i.e., at the 2 hr time point following each injection). (FIG. 3). In Cluster 7, twenty genes were identified, including alkaline phosphatase, c-fos, collagenase 3 (see FIG. 4), RoBo-1, and four novel genes.

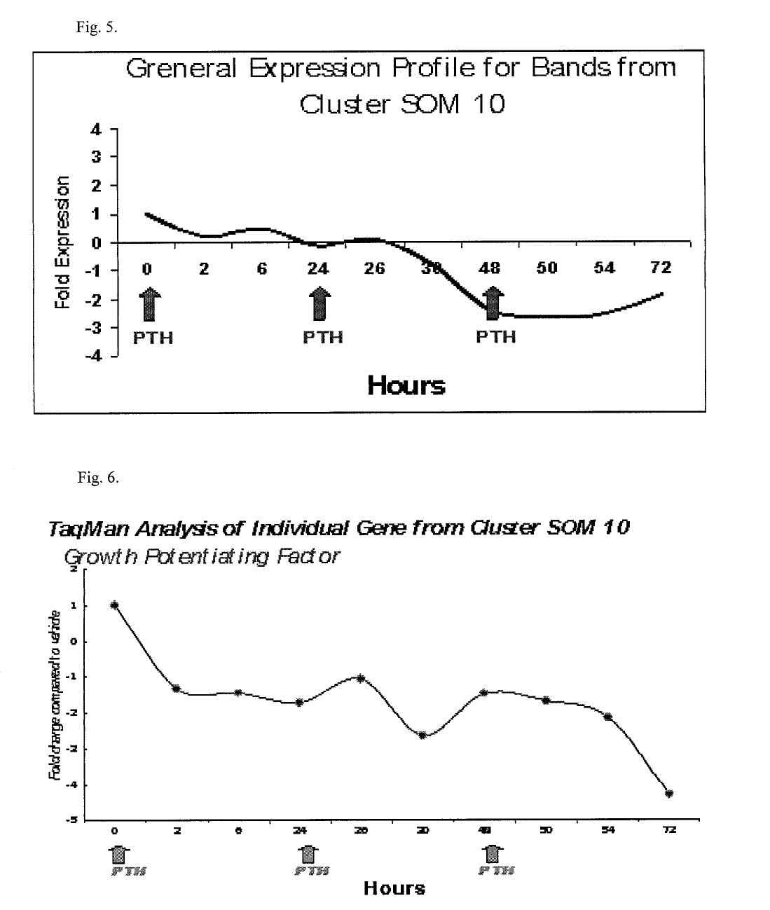

Bands in SOM10 showed a decrease in expression beginning at day 2, 24 hours and maintained decreased expression in all subsequent time points (FIG. 5). In Cluster 10, 24 genes were identified, including carbonic anhydrase, cathepsin E, growth potentiating factor (see FIG. 6), B-cell Translocating Gene 1 (BTG 1), and seven novel genes.

Intermittent PTH administrations up regulated expression of a number of genes which are either known markers of differentiated osteoblasts or indicative of increase in osteoblast function and bone mineralization. These data strongly support the finding that intermittent injection of parathyroid hormone stimulates bone formation activity of osteoblast.

### Example 2

### Quantitative Analysis of OST mRNA

A quantitative assay to detect mRNA was designed. The assay used real-time fluorescent-probe polymerase chain reaction (PCR) (TaqMan).

The TaqMan sequence detector (ABI PRISM 7700 Sequence Detector) integrates a PCR-based assay and hardware/software instrumentation to provide high-throughput quantitation of nucleic acid sequences. The combination of thermal cycling, fluorescence detection, and application-specific software allows the cycle-by-cycle detection of the increase in the PCR product. Quantitative results are provided.

The method used a fluorogenic probe complementary to the target sequence, which was added to the PCR reaction mixture. The probe comprised an oligonucleotide with a reporter and quencher dye attached. If the target nucleic acid was present during PCR, the probe annealled specifically between the forward and reverse primer sites. The polymerase cleaved the probe, releasing the reporter dye from the influence of the quencher dye, and thus causing an increase in the fluorescent intensity of the reporter dye. Fluorescent emission was then recorded and/or measured. See, e.g., Orlando, C., P., Pinzani, and M., Pazzagli. “Developments in quantitative PCR.” Clin Chem Lab Med 36 (1998): 255-269; Brink et al., “Comparative quantification of IL-1beta, IL-10, IL-10r, TNFalpha and IL-7 mRNA levels in UV-irradiated human skin in vivo”, Inflamm Res 49(6):290 (2000).

The amount of mRNA encoding the following proteins was assessed in the rat bone samples as described in Example 1: collagenase 3, RoBo-1, Ryudocan, Growth Potentiating Factor; bone sialoprotein, brain creatine kinase, cathepsin, osteonectin, and TIMP-1.

Results are shown in the following tables, where “Ct” refers to the number of PCR cycles required to produce a threshold amount of product. Table 3 compares the number of PCR cycles needed for control and treatment samples to reach the threshold amount of mRNA (delta Ct); Table 4 compares the amount of mRNA in control and treatment samples.

TABLE 3
Delta Ct Values Characterizing Changes in Gene Expression

| | | | | | Bone | | | | |
|---|---|---|---|---|---|---|---|---|---|
| | Collagenase | RoBo-1 | Ryudocan | GPF | Sialo | BCKinase | Cathepsin | Osteonec | TIMP-1 |
| Day 1 PTH | 0.5 | −0.1 | 2.6 | −0.4 | −0.5 | −0.3 | 0.0 | −0.5 | 2.6 |
| 2 HR | | | | | | | | | |
| Day 1 PTH | 0.4 | −0.1 | 1.1 | −0.5 | 0.5 | −0.1 | 0.1 | −0.1 | 3.1 |
| 6 HR | | | | | | | | | |
| Day 1 PTH | −1.4 | −0.3 | −0.6 | −0.8 | −0.3 | −0.5 | −0.5 | −0.6 | 1.6 |
| 24 HR | | | | | | | | | |
| Day 2 PTH | 0.5 | −0.2 | 3.6 | −0.1 | −0.1 | −0.1 | −0.3 | −0.1 | 3.7 |
| 2 HR | | | | | | | | | |
| Day 2 PTH | 0.1 | −0.2 | 0.6 | −1.4 | 0.1 | −0.1 | −0.8 | −0.7 | 3.5 |
| 6 HR | | | | | | | | | |
| Day 2 PTH | −0.3 | 0.9 | 0.1 | −0.6 | 1.3 | 0.3 | −0.3 | 0.1 | 2.4 |
| 24 HR | | | | | | | | | |
| Day 3 PTH | 1.2 | 0.0 | 3.1 | −0.8 | 0.4 | 0.1 | −0.5 | −0.2 | 4.1 |
| 2 HR | | | | | | | | | |
| Day 3 PTH | 2.5 | 0.3 | 0.5 | −1.1 | 0.4 | 0.1 | −0.8 | −0.3 | 4.2 |
| 6 HR | | | | | | | | | |
| Day 3 PTH | −0.1 | −0.6 | −1.3 | −2.1 | −0.8 | −2.0 | −1.6 | −1.4 | 1.9 |
| 24 HR | | | | | | | | | |

TABLE 4
Fold Change in Gene Expression

| | | | | | Bone | | | | |
|---|---|---|---|---|---|---|---|---|---|
| | Collagenase | RoBo-1 | Ryudocan | GPF | Sialo | BCKinase | Cathepsin | Osteonec | TIMP-1 |
| Day 1 PTH | 1.44 | −1.08 | 6.21 | −1.32 | −1.45 | −1.21 | 1.02 | −1.38 | 6.00 |
| 2 HR | | | | | | | | | |
| Day 1 PTH | 1.34 | −1.07 | 2.12 | −1.43 | 1.39 | −1.10 | 1.05 | −1.10 | 8.75 |
| 6 HR | | | | | | | | | |
| Day 1 PTH | −2.68 | −1.25 | −1.52 | −1.72 | −1.23 | −1.42 | −1.39 | −1.54 | 3.05 |
| 24 HR | | | | | | | | | |
| Day 2 PTH | 1.44 | −1.12 | 11.79 | −1.06 | −1.10 | −1.07 | −1.27 | −1.08 | 12.95 |
| 2 HR | | | | | | | | | |
| Day 2 PTH | 1.05 | −1.14 | 1.49 | −2.65 | 1.11 | −1.07 | −1.80 | −1.62 | 11.20 |
| 6 HR | | | | | | | | | |
| Day 2 PTH | −1.24 | 1.91 | 1.06 | −1.48 | 2.48 | 1.24 | −1.27 | 1.04 | 5.30 |
| 24 HR | | | | | | | | | |
| Day 3 PTH | 2.29 | 1.03 | 8.82 | −1.68 | 1.36 | 1.09 | −1.44 | −1.14 | 17.51 |
| 2 HR | | | | | | | | | |
| Day 3 PTH | 5.76 | 1.23 | 1.42 | −2.15 | 1.28 | 1.06 | −1.74 | −1.24 | 18.90 |
| 6 HR | | | | | | | | | |
| Day 3 PTH | −1.05 | −1.52 | −2.38 | −4.30 | −1.72 | −3.99 | −3.11 | −2.56 | 3.71 |
| 24 HR | | | | | | | | | |

Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method of identifying a ligand for the parathyroid hormone receptor, the method comprising;
(a) providing a test cell population comprising a cell capable of expressing one or more nucleic acid sequences selected from the group consisting of OST: 1-47 and 48;
(b) contacting the test cell population with a test agent;
(c) measuring expression of one or more of the nucleic acid sequences in the test cell population;
(d) comparing the expression of the nucleic acid sequences in the test cell population to the expression of the nucleic acid sequences in a reference cell population comprising at least one cell whose parathyroid hormone status is known; and
(e) identifying a difference in expression levels of the OST sequence, if present, in the test cell population and reference cell population, thereby identifying a ligand for the parathyroid hormone receptor.

2. The method of claim 1, wherein the method comprises comparing the expression of five or more of the nucleic acid sequences.

3. The method of claim 1, wherein the method comprises comparing the expression of 20 or more of the nucleic acid sequences.

4. The method of claim 1, wherein the method comprises comparing the expression of 25 or more of the nucleic acid sequences.

5. The method of claim 1, wherein the expression of the nucleic acid sequences in the test cell population is decreased as compared to the reference cell population.

6. The method of claim 1, wherein the expression of the nucleic acid sequences in the test cell population is increased as compared to the reference cell population.

7. The method of claim 1, wherein the test cell population is provided in vitro.

8. The method of claim 1, wherein the test cell population is provided ex vivo from a mammalian subject.

9. The method of claim 1, wherein the test cell is provided in vivo in a mammalian subject.

10. The method of claim 1, wherein the test cell population is derived from a human or rodent subject.

11. The method of claim 1, wherein the test cell includes a bone cell.

12. The method of claim 11, wherein the bone cell is an osteoclast or osteoblast.

13. A parathyroid hormone receptor ligand identified according to the method of claim 1.

14. A pharmaceutical composition comprising the parathyroid hormone receptor ligand of claim 13.

15. A method of screening for an osteoregenerative agent, the method comprising;
(a) providing a test cell population comprising a cell capable of expressing one or more nucleic acid sequences selected from the group consisting of OST: 1-1-47 and 48;
(b) contacting the test cell population with a test agent;
(c) measuring expression of one or more of the nucleic acid sequences in the test cell population;
(d) comparing the expression of the nucleic acid sequences in the test cell population to the expression of the nucleic acid sequences in a reference cell population comprising at least one cell whose osteoregenerative agent exposure status to is known; and
(e) identifying a difference in expression levels of the OST sequence, if present, in the test cell population and reference cell population,
thereby screening for an osteogenerative agent.

16. The method of claim 15, wherein the method comprises comparing the expression of 40 or more of the nucleic acid sequences.

17. The method of claim 15, wherein the expression of the nucleic acid sequences in the test cell population is decreased as compared to the reference cell population.

18. The method of claim 15, wherein the expression of the nucleic acid sequences in the test cell population is increased as compared to the reference cell population.

19. The method of claim 15, wherein the test cell population is provided in vitro.

20. The method of claim 15, wherein the test cell population is provided ex vivo from a mammalian subject.

21. The method of claim 15, wherein the test cell population is provided in vivo in a mammalian subject.

22. The method of claim 15, wherein the test cell population is derived from a human or rodent subject.

23. The method of claim 15, wherein the test cell population includes a bone cell.

24. A method of assessing the osteoregenerative activity of a test agent in a subject, the method comprising:
(a) providing from the subject a test cell population comprising a cell capable of expressing one or more nucleic acid sequences selected from the group consisting of OST: 1-47 and 248;
(b) contacting the test cell population with a test agent;
(c) measuring expression of one or more of the nucleic acid sequences in the test cell population; and
(d) comparing the expression of the nucleic acid sequences in the test cell population to the expression of the nucleic acid sequences in a reference cell population comprising at least one cell whose osteoregenerative agent exposure status is known;
(e) identifying a difference in expression levels of the nucleic acid sequences, if present, in the test cell population and the reference cell population,
thereby assessing the osteoregenerative activity of the test agent in the subject.

25. The method of claim 24, wherein the expression of the nucleic acid sequences in the test cell population is decreased as compared to the reference cell population.

26. The method of claim 24, wherein the expression of the nucleic acid sequences in the test cell population is increased as compared to the reference cell population.

27. The method of claim 24, wherein said subject is a human or rodent.

28. The method of claim 24, wherein the test cell population is provided ex vivo from said subject.

29. The method of claim 24, wherein the test cell population is provided in vivo from said subject.

30. A method of diagnosing or determining the susceptibility to a bone disorder in a subject, the method comprising:
(a) providing from the subject a test cell population comprising cells capable of expressing one or more nucleic acid sequences selected from the group consisting of OST:1-47 and 48;
(b) measuring expression of one or more of the nucleic acid sequences in the test cell population; and
(c) comparing the expression of the nucleic acid sequences in the test cell population to the expression of the nucleic acid sequences in a reference cell population comprising at least one cell from a subject not suffering from a bone disorder; and
(d) identifying a difference in expression levels of the nucleic acid sequences, if present, in the test cell population and reference cell population,
thereby diagnosing or determining the susceptibility to a bone disorder in the subject.

31. A method of treating a bone disorder in a subject, the method comprising administering to the subject an agent that modulates the expression or the activity of one or more nucleic acids selected from the group consisting of OST: 1-47 and 48.

32. The method of claim 31, wherein the bone disorder is a bone formation disorder.

33. The method of claim 31, wherein the bone disorder is a bone resorbtion disorder.

34. The method of claim 31, wherein the bone disorder is a selected from the group comprising osteoporosis, osteopenia and osteopetrosis.

35. A method of assessing the efficacy of a treatment of a bone disorder in a subject, the method comprising:
(a) providing from the subject a test cell population comprising cells capable of expressing one or more nucleic acid sequences selected from the group consisting of OST: 1-47 and 48;
(b) detecting expression of one or more of the nucleic acid sequences in the test cell population;
(c) comparing the expression of the nucleic acid sequences in the test cell population to the expression of the nucleic acid sequences in a reference cell population comprising at least one cell from a subject not suffering from the bone disorder; and
(e) identifying a difference in expression levels of the nucleic acid sequences, if present, in the test cell population and reference cell population,
thereby assessing the efficacy of treatment of the bone disorder in the subject.

36. An isolated nucleic acid comprising a nucleic acid sequence selected from the group consisting of a OST 1-32 gene, or its complement.

37. A vector comprising the nucleic acid of claim 36.

38. A cell comprising the vector of claim 37.

39. A pharmaceutical composition comprising the nucleic acid of claim 36.

40. A polypeptide encoded by the nucleic acid of claim 36.

41. A polypeptide comprising the amino acid sequence of SEQ ID NO: 33-53.

42. An antibody which specifically binds to the polypeptide of claim 41.

43. A kit which detects two or more of the nucleic acid sequences selected from the group consisting of OSTs: 1-48.

44. An array which detects one or more of the nucleic acid selected from the group consisting of OSTs: 1-48.

45. A plurality of nucleic acid comprising one or more of the nucleic acid selected from the group consisting of OSTs: 1-48.
